(19) United States
(12) Patent Application Publication
(10) Pub. No.: US 2026/0243757 A1
(43) Pub. Date: Aug. 20, 2026

(54) IMMUNE CELL-DERIVED SECRETOME

(71) Applicants: Agency for Science, Technology and Research, Singapore (SG); Singapore Health Services Pte Ltd, Singapore (SG)

(72) Inventor: Wan Yee (Wanyi) Teo (Zhang), Singapore (SG)

(21) Appl. No.: 19/160,643

(22) PCT Filed: Feb. 28, 2024
(86) PCT No.: PCT/SG2024/050115

§ 371 Date: Aug. 28, 2025

(30) Foreign Application Priority Data
Feb. 28, 2023 (SG) 10202300541U

(51) Int. Cl. G01N 33/50; C07K 14/52; C07K 14/54; C07K 14/715; C12N 5/078

(52) U.S. Cl. CPC G01N 33/5047; C07K 14/52; C07K 14/523; C07K 14/5421; C07K 14/7155; C12N 5/0634; C12N 2501/2304; C12N 2501/999; G01N 2500/10

(57) ABSTRACT

There is provided a composition comprising a secretome obtained by culturing an immune progenitor cell in the presence of an agent that activates the immune cell progenitor to an activated immune cell. Also provided is a composition for use in tissue regeneration, a method of generating a macrophage-derived secretome, a method of screening a drug and a method of culturing a proliferative cell.

### TECHNICAL FIELD

The present disclosure relates broadly to immune cell-derived secretome. In particular, the present disclosure relates to a composition comprising a secretome obtained by culturing a cell in the presence of an immune cell.

### BACKGROUND

Cancer cells need nutrients to grow and proliferate. During periods of nutrient stress in the microenvironment, it is not well understood if or how cancer cells can adopt alternative resources to re-wire and survive in patients. Patient tumours are more complex than in-vitro culture systems. Patient tumours comprise of a microenvironment surrounding cancer cells. Nonetheless, laboratory testing of drugs still uses in-vitro cancer cell cultures without a microenvironment, which remains an imperfect strategy for drug discovery and translation in oncology.

Clinical implications of immune cell/macrophage density infiltrating tumours vary between different body organs. Increased immune cell/macrophage density has been associated with tumour progression and/or negative clinical outcome in some organs (brain, breast, liver, kidney, ovary, pancreas, thyroid, head and neck. In colorectal cancer, increased immune cell/macrophage infiltration was linked to better prognosis, while cancers of the bone, lung and prostate, the role of immune cell/macrophages remain controversial. The complexity of the problem is multiplied by the myriad of dynamic immune cell/macrophage polarization spectrum. Over the last decade, infiltrating immune cell/macrophages are increasingly recognized to predominate brain tumours, but there is still no consensus on the clinical significance of immune cell/macrophage infiltration relevant to patient outcome in brain tumours. Some current strategies to study immune cell/macrophages infiltrating patient tumours include genomic profiling of micro-dissected regions of immune cell/macrophages in patient tumours, and single cell sequencing of individual immune cell/macrophages in patient tumours. These approaches involve tearing apart of the immune cell/macrophage microenvironment compartment from the tumour cell compartment of patient tumours harvested. Newer approaches such as spatial transcriptomics, allow gene expression profiling without disrupting the tumour architecture. However, the common challenge for all these approaches, is that the resected patient tumours are immortalized at a single time-point. The ability to study the in-situ impact of macrophage presence over time, is lost.

Therefore, there is a need to provide an alternative method to study the effects of immune cell/macrophage on the tumour microenvironment.

### SUMMARY

In one aspect, there is provided a composition comprising a secretome obtained by culturing an immune progenitor cell in the presence of an agent that activates the immune progenitor cell to an activated immune cell.

In some examples, the secretome comprises a protein comprising a cytokine, a hormone, an antibody, a growth factor, an extracellular matrix protein, a shed receptor, a coagulation factor, an adhesion molecule, a protease, a kinase and/or a glycoprotein.

In some examples, the secretome comprises one or more factors selected from the group consisting of CCL2/MCP-1, MIP-1α/MIP-1β, CCL5/RANTES, MIF, IL-1ra/IL-IF3, IL-8and Serpin E1/PAI-1.

In some examples, the immune cell comprises a myeloid cell, a lymphoid cell, and/or an innate immune cell.

In some examples, the immune progenitor cell is a myeloid progenitor cell.

In some examples, the immune cell is a macrophage expressing CD11b and/or CD163.

In some examples, the secretome is a macrophage-derived secretome.

In some examples, the agent comprises a stimulating agent or a suppressing agent.

In some examples, the agent is a myeloid stimulating agent.

In some examples, the agent is PMA and/or IL-4.

In some examples, the secretome sustains cell growth, cell proliferation, cryopreserve cells, elongates telomere length in cancer cells, involved in metabolic reprogramming, identifying drug resistance and/or preventing cancer cell death under nutrient stress conditions.

In another aspect, there is provided a composition for use in tissue regeneration.

In yet another aspect, there is provided a method of generating a macrophage-derived secretome comprising culturing an immune progenitor cell in the presence of a stimulating agent to thereby generate an activated immune cell population and harvesting the secretome from the generated activated immune cell population.

In some examples, the method comprises culturing the activated immune cell in the presence of a diseased cell.

In some examples, the diseased cell is a cell from a proliferative disease.

In some examples, the diseased cell is a brain tumour, an atypical teratoid rhabdoid tumour (ATRT) and/or medulloblastoma.

In yet another aspect, there is provided a method of screening a drug comprising culturing the drug with diseased cell in the presence of the composition as disclosed herein.

In yet another aspect, there is provided a method of culturing a proliferative cell comprising culturing the proliferative cell in a composition as disclosed herein.

### DESCRIPTION OF EMBODIMENTS

The tumour microenvironment includes diverse cell types such as immune cells, cancer-associated fibroblasts, endothelial cells, pericytes, and various additional tissue-resident cell types. These host cells are known to play critical roles in the pathogenesis of cancer e.g., increased immune cell/macrophage density has been associated with tumour progression and negative clinical outcome. In view of the importance of the role of the tumour microenvironment in the pathogenesis of cancer, there is a need to provide methods to understand the role/effects of immune cell/macrophages in the tumour microenvironment. The present disclosure provides a method to study the role/effects of immune cell/macrophages in the tumour microenvironment that addressed at least one of the problems currently faced in the study of immune cell/macrophages in a tumour/tumour microenvironment.

In one aspect, there is provided a composition comprising a secretome obtained by culturing an immune progenitor cell in the presence of an agent that activates the immune progenitor cell to an activated immune cell.

In some examples, the immune progenitor cell is a myeloid progenitor cell.

In some examples, there is provided a composition comprising a secretome obtained by culturing a cell in the presence of an agent to generate an immune cell.

In some examples, there is provided a composition comprising a secretome obtained by culturing a cell in the presence of an agent to generate an immune cell, wherein the immune cell is a cell capable of expressing CD11b.

In some examples, the cell is a cell line.

In some examples, the cell line may comprise but is not limited to, a myeloid cell line, a lymphoma cell line, and the like.

In some examples, the cell line may include but is not limited to, human myeloid leukaemia derived cell line (U937), human lymphoma cell line (LK46), THP1, and the like.

In some examples, the cell line is a human myeloid leukaemia derived cell line (U937).

As used herein, the term “secretome” refers to the totality of released molecules that are organic and inorganic components by biologic cells, tissues and/or organs. The secretome is the set of components expressed by biologic cells, tissues and/or organs and secreted into extracellular space which includes paracrine substances, exosomes and microvesicles.

In some examples, the secretome may include components such as but is not limited to, polypeptide, polynucleotide, lipid, carbohydrate, exosomes, microvesicles, paracrine substances, and the like.

In some examples the polynucleotide may include but is not limited to, DNA, mRNA, miRNA, non-coding RNA, and the like.

In some examples the secretome may comprise a protein such as but is not limited to, a cytokine, a hormone, an antibody, a growth factor, an extracellular matrix protein, a shed receptor, a coagulation factor, an adhesion molecule, a protease, a kinase, a glycoprotein, a protease inhibitor, and the like.

In some examples, the composition includes cytokines such as chemotactic cytokines, chemokines, and lymphokines.

As used herein, the term “cytokine” includes chemokine, chemotactic cytokines and lymphokines. Chemokines/chemotactic cytokines such as CCL14, CCL2, CCL19, CCL5, and the like, are a family of small cytokines or signalling proteins secreted by cells that induce directional movement of leukocytes, as well as other cell types, including endothelial and epithelial cells. Lymphokines refer to a subset of cytokines that are produced by lymphocytes. Their role includes attracting other immune cells including macrophages and other lymphocytes to an infected site and their subsequent activation to prepare them to mount an immune response. Lymphokines that are secreted by T helper cells include for example IL-2, IL-3, IL-4, and the like.

In some examples, cytokines may include pro-inflammatory and/or anti-inflammatory cytokines.

In some examples, proinflammatory cytokines may include but is not limited to, Chemokine ligand 5 (CCL5/RANTES), Macrophage inflammatory protein-1α/Macrophage inflammatory protein-1β (MIP-1α/MIP-1β), Macrophage migration Inhibitory Factor (MIF), Interleukin-8 (IL-8), Monocyte Chemoattractant Protein-1 (CCL2/MCP-1), IL-6, TNF-α, IL-1β and the like.

In some examples, anti-inflammatory cytokines may include but is not limited to, IL-1 receptor antagonist (IL-1ra/IL-IF3), IL-4, IL-10, IL-11, IL-13, and the like. In some examples, the composition may include a protease inhibitor.

In some examples, the composition may include a serine protease inhibitor such as Serpin E1/PAI-1.

In some examples, the composition may comprise one-factor, two-factors, three-factors, four-factors, five-factors, six-factors, seven-factors, eight-factors, nine-factors or 10-factors.

As used herein, the term “a factor” refers to an individual component of a secretome.

In some examples, the composition comprises six-factors. In some examples, the composition comprises seven-factors.

In some examples, the secretome comprises one or more factors selected from the group consisting of CCL2/MCP-1, MIP-1α/MIP-1β, CCL5/RANTES, MIF, IL-1ra/IL-IF3, IL-8 and/or Serpin E1/PAI-1.

In some examples, the secretome comprises one or more factors selected from the group consisting of CCL2/MCP-1, MIP-1α/MIP-1β, CCL5/RANTES, MIF, IL-1ra/IL-IF3 and IL-8.

In some examples, the factors include CCL2/MCP-1, MIP-1α/MIP-1β, CCL5/RANTES, MIF, IL-1ra/IL-IF3, and/or IL-8.

In some examples, the composition obtained from an immune cell (such as a macrophage) may include but is not limited to, CCL5/RANTES, IL-1ra/IL-IF3, MIP-1α/MIP-1β,MIF, IL-8, CCL2/MCP-1, Serpin E1/PAI-1 (from highest to lowest expression) and the like.

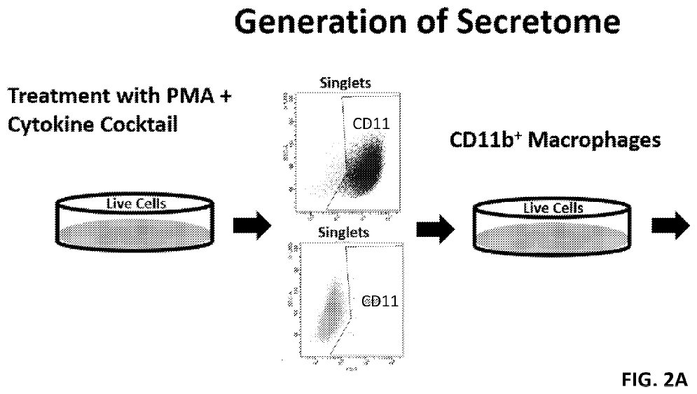

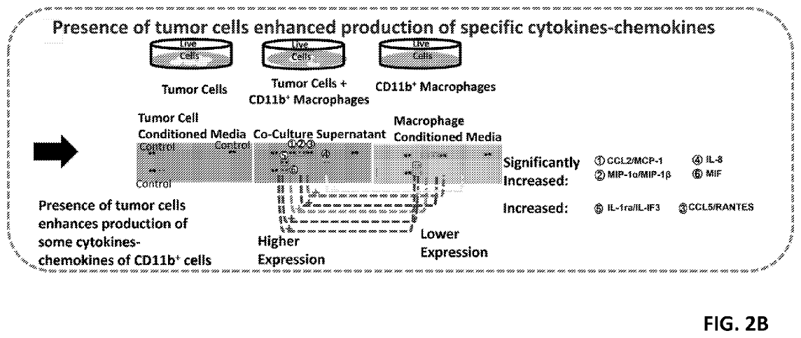

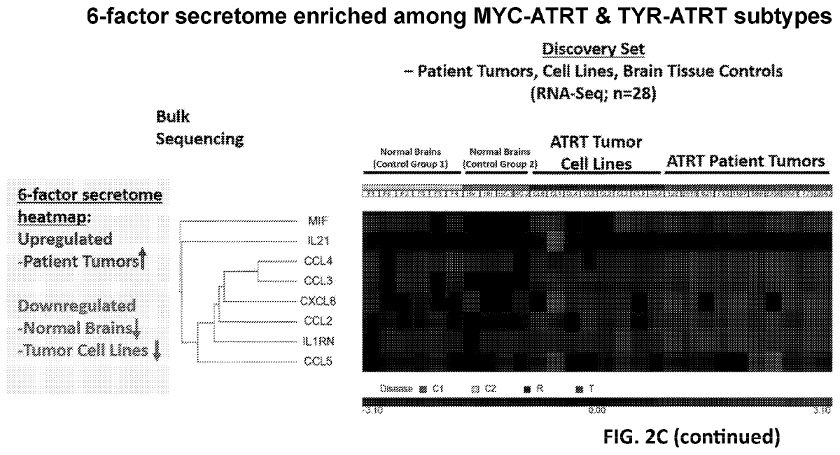

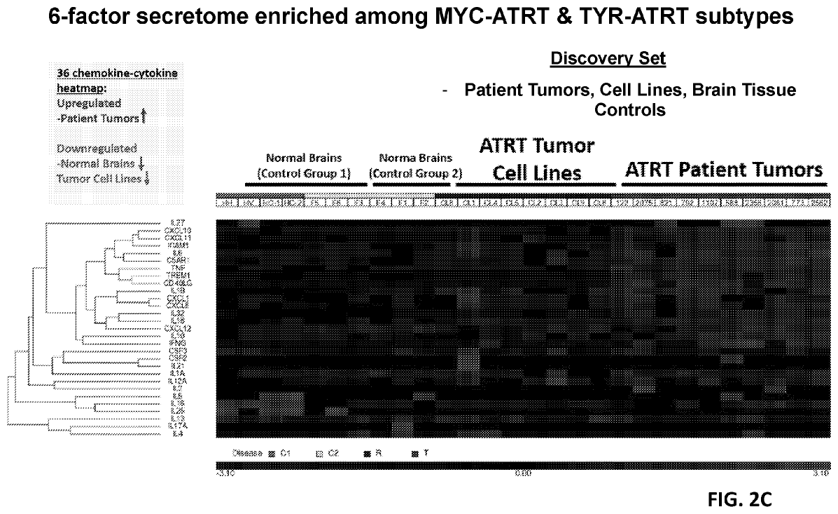

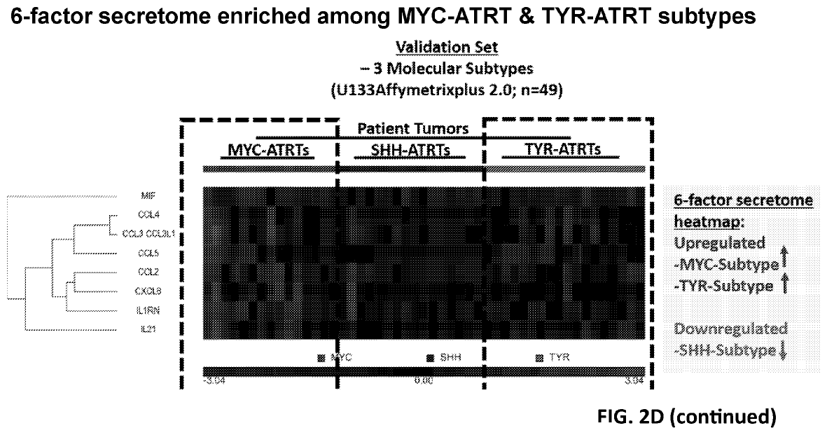

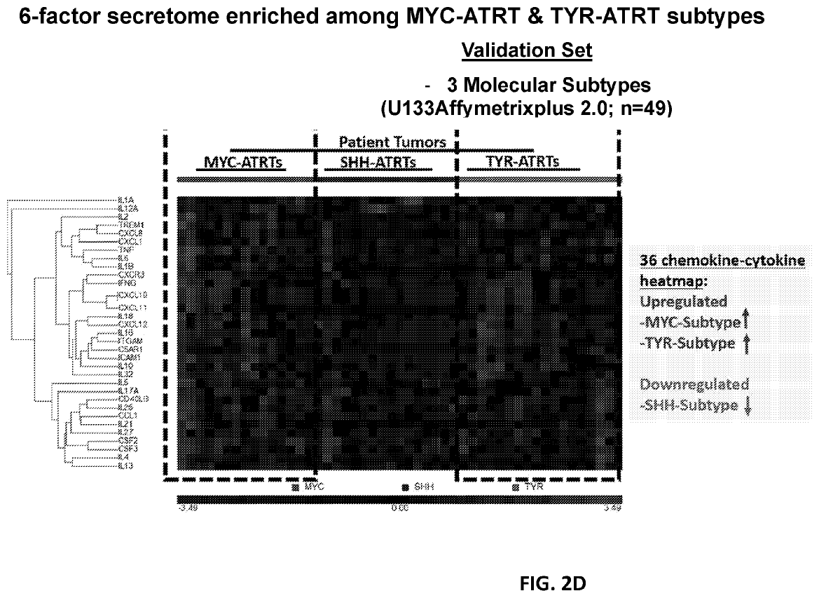

In some examples, the composition obtained from an immune cell (such as a macrophage/a macrophage expressing CD11b) in the presence of a diseased cell (such as tumour cell) may include significantly increased expression of CCL-2/MCP-1, MIP-1α/MIP-1β,IL-8, MIF; and increased expression of IL-1ra/IL-IF3, CCL-5/RANTES and Serpin E1/PAI-1.Advantageously, the present disclosure comprises a secretome product that comes in a mix of important factors and at a biological dose of each factor, which is biologically determined by stimulated macrophages in laboratory culture systems. This is different from most cytokines/chemokines in the market that are produced and packaged singly. As shown in FIG. 2A to 2D, the biological dose of each of the factors were produced in quantities large enough to be detected on standard culture assays such as, but is not limited to a cytokine blot, RNA sequencing, microarray, and the like.

The secretome during nutrient stress is involved in cancer growth/proliferation and in preventing cancer cell death. The secretome is superior to standard (serum-based or serum-free) growth media at 4 to 96 hours in promoting cancer growth/proliferation and is equal to standard media in preventing cancer cell death at 4 to 96 hours.

The secretome is also involved in drug response of cancer cells. In standard media, drug is rapidly efficacious. The drug response for the same drug in secretome is dampened. This indicates that a drug which is effective in standard media during in-vitro testing in the laboratory, may not be equally efficacious in patient tumours which harbour immune-rich conditions. This can result in failure of drugs when translated at clinical trial stage because patient tumours can harbour immune-rich microenvironment. Some drugs can cause cancer cell death in-vitro more effectively in secretome compared to standard media. The ability to identify drugs which can cause cancer cell death in patient tumours which are rich in immune conditions is important in tailoring precision drugs for patients.

In some examples, the drug response for a diseased cell (such as medulloblastoma cells) may be more sensitive in the presence of the secretome as compared to standard media.

In some examples, the number of immune progenitor cell cultured/seeded with an may include at least 50 cells, at least 100 cells, at least 150 cells, at least 200 cells, at least 250 cells, at least 300 cells, at least 350 cells, at least 400 cells, at least 450 cells, at least 500 cells, at least 550 cells, at least 1000 cells, at least 1500 cells, at least 2000 cells, at least 2500 cells, at least 3000 cells, at least 3500 cells, at least 4000 cells, at least 4500 cells, at least 5000 cells, at least 5500 cells, at least 6000 cells, at least 6500 cells, at least 7000 cells, at least 7500 cells, at least 8000 cells, at least 8500 cells, at least 9000 cells, at least 9500 cells, at least 10,000 cells, at least 20,000 cells, at least 30,000 cells, at least 40,000 cells, at least 50,000 cells, at least 60,000 cells, at least 70,000 cells, at least 80,000 cells, at least 90,000 cells, at least 100,000 cells, at least 150,000 cells, at least 200,000 cells, at least 250,000 cells, at least 300,000 cells, at least 350,000 cells, at least 400,000 cells, at least 450,000 cells, at least 500,000 cells, at least 550,000 cells, at least 600,000 cells, at least 650,000 cells, at least 700,000 cells, at least 750,000 cells, at least 800,000 cells, at least 850,000 cells, at least 900,000 cells, at least 1,000,000 cells, and the like.

In some examples, the number of immune progenitor cell that are cultured/seeded may include about 50 cells, about 100 cells, about 150 cells, about 200 cells, about 250 cells, about 300 cells, about 350 cells, about 400 cells, about 450 cells, about 500 cells, about 550 cells, about 1000 cells, about 1500 cells, about 2000 cells, about 2500 cells, about 3000 cells, about 3500 cells, about 4000 cells, about 4500 cells, about 5000 cells, about 5500 cells, about 6000 cells, about 6500 cells, about 7000 cells, about 7500 cells, about 8000 cells, about 8500 cells, about 9000 cells, about 9500 cells, about 10,000 cells, about 20,000 cells, about 30,000 cells, about 40,000 cells, about 50,000 cells, about 60,000 cells, about 70,000 cells, about 80,000 cells, about 90,000 cells, about 100,000 cells, about 150,000 cells, about 200,000 cells, about 250,000 cells, about 300,000 cells, about 350,000 cells, about 400,000 cells, about 450,000 cells, about 500,000 cells, about 550,000 cells, about 600,000 cells, about 650,000 cells, about 700,000 cells, about 750,000 cells, about 800,000 cells, about 850,000 cells, about 900,000 cells, about 1,000,000 cells, and the like.

In some examples, the immune progenitor cell may be cultured in a petri-dish, 6 well plate, a 12 well plate, a 24 well plate, a 96 well plate, a T-25 flask, a T-75 flask, and the like.

In some examples, the immune progenitor cell may be cultured in a dish/flask/plate that is made of such as but is not limited to glass, plastic, and the like.

In some examples, the immune cell may comprise but is not limited to a myeloid cell, a lymphoid cell, an innate immune cell, and the like.

In some examples, the immune cell may include but is not limited to a cell expressing CD11b, CD163, and the like.

In some examples, the immune cell is capable of expressing CD11b.

In some examples, the immune cell is a cell expressing CD11b. In some examples, the immune cell may be an immune cell that upon maturation or activation would express CD11b.

In some examples, the immune cell is a macrophage.

In some examples, the macrophage may include any macrophage cell marker, but is not limited to a cell expressing CD11b, CD14, CD16, CD64, CD163, CD68, MARCO, and the like.

In some examples, the immune cell is a macrophage expressing CD11b.

In some examples, the immune cell is a macrophage expressing CD11b and/or CD163.

In some examples, a myeloid cell may include a cell derived from a myeloid progenitor cell, such as, but is not limited to, a macrophage, a monocyte, a dendritic cell, an antigen presenting cell, and the like.

In some examples, the myeloid cell may include any cell expressing a myeloid cell marker, such as, but is not limited to a cell expressing CD11b, CD206, CD68, CD15, and the like.

In some examples, a lymphoid cell may include, but is not limited to, a T lymphocyte, a B lymphocyte, and the like.

In some examples, the lymphocyte may include but is not limited to a cell expressing CD3, CD4, CD8, and the like.

In some examples, the innate immune cell may include, but is not limited to, a neutrophil, a natural killer cell, and the like.

In some examples, the neutrophil may include but is not limited to a cell expressing CD16, CD15, CD10, CXCR2, and the like.

In some examples, the secretome is a macrophage-derived secretome.

Without wishing to be bound by theory, macrophages have been observed to infiltrate many human tumours and across cancer types. Macrophages in the stroma of patient tumours can be found in large quantities and are therefore highly attractive and desirable targets in the microenvironment. However, macrophages are dynamic, and offer many facets and phenotypes which the field has yet to fully comprehend. In the present disclosure, instead of macrophages, the inventor of the present disclosure approached the secretome of macrophages in the microenvironment of patient tumours, which offers the benefit and advantages of examining a more constant environmental fluid surrounding tumour cell. The inventor of the present disclosure hypothesized that various cytokines-chemokines from the secretome derived from macrophages continuously provide a critical niche for tumour progression/development and impact drug response of each individual tumour. In the absence of nutrients during nutrient stress, macrophages may provide this secretome as an alternative fuel source for cancer cells to survive. To explore the real-time, dynamic interaction between the macrophage-tumour ecosystem, the inventor of the present disclosure developed an assay utilizing secretome derived from human CD11b+ (pan-macrophage) cells to establish its effects on atypical teratoid rhabdoid tumour (ATRT) and medulloblastoma which are both pediatric embryonal brain tumours.

In some examples, the secretome is a cell-free macrophage-derived secretome.

In some examples, the cell-free macrophage derived secretome is obtained from human and non-human organism. In some examples, a non-human organism may include but is not limited to, mice, rat, non-human primates, and the like. In some examples, the background of a mice may include but is not limited to, C57/BL6, BALB/c, CD-1, SCID, and the like. In some examples, the background of a rat may include but is not limited to, A/J, Sprague Dawley, Wistar, and the like. In some examples, non-human primates may include but is not limited to, Rhesus monkey, Japanese monkey, Olive baboon, Squirrel monkey, Capuchin monkey, and the like.

In some examples, the cell-free macrophage derived secretome is obtained from a human and non-human organism that is enriched in CD11b. In some examples, the cell-free macrophage derived secretome is obtained from a non-human organism that is enriched in CD11b. In some examples, the cell-free macrophage derived secretome is obtained from a mice that is enriched in CD11b. In some examples, the mice that is enriched in CD11b may include but is not limited to a counterpart of a SCID/RAG2 immunocompromised/immunosuppressed mice, and the like.

In some examples, the secretome is a cell-free human macrophage-derived secretome.

Without wishing to be bound by theory, cancer cells need nutrients to grow and proliferate. In the absence of nutrients during nutrient stress in the microenvironment, it is not well understood if or how cancer cells can adopt alternative resources to re-wire and survive in patients. Patient tumours are more complex than in-vitro culture systems. Patient tumours comprise of microenvironment surrounding cancer cells. Laboratory testing of drugs using media-based in-vitro cultures, remains an imperfect strategy for drug discovery and translation.

The approach of the present disclosure targets cancer cells in their microenvironment conditions. Macrophages are difficult to study as they encompass a diverse spectrum and not all phenotypes are well characterized. The inventor of the present disclosure uses the secretome of macrophages which provides the surrounding microenvironmental milieu of tumour cells. This allows the effects of secretome (derived from macrophages) surrounding cancer cells to be studied. Studies known in the art that study macrophage infiltrating patient tumours involve tearing apart of the macrophage microenvironment compartment from the tumour cell compartment/macrophages/immune cells away from tumour cells, immortalized at a single time point. Advantageously, the present disclosure circumvents this problem/addressed this research challenge using a secretome-based approach. This allows the effects of the secretome of macrophages surrounding cancer cells to be studied mechanistically in-situ, with dynamic in-situ interaction between the two-compartment macrophage tumour ecosystems, and without disrupting the microenvironment milieu. In addition, studies in the art typically investigate the effect of factors on cancer cell individually (i.e. investigate one at a time). In contrast, the present disclosure harvested a bag of several factors in the tumour cell milieu relevant to patient tumours, which are secreted at biological quantities by CD11b+ cells. The effects of the biologically relevant immune milieu on in-vitro and in-vivo screens on cancer cell survival in the state of nutrient depletion (such as stress) and drug response is established in the present disclosure. This provides high commercial value and research asset and is highly applicable for the pharmaceutical industry. Also, the secretome which is derived from human macrophages makes it more patient relevant for drug screening.

In addition, effects of immune/macrophage microenvironment in modulating drug response in patient tumours is less understood. A drug which is effective in-vitro in the laboratory, when administered to patients, will likely be impacted by the macrophage/immune microenvironment within patient tumours (these are absent in-vitro on petri-dishes of cancer cells). Therefore, by providing a macrophage-derived secretome to cancer cells during high throughput drug screening in the laboratory, the inventor of the present disclosure is able to objectively measure the impact of secretome on drug efficacy, which is relevant to translating this application to patient tumours which are more immune-rich than cancer cells on petri-dishes devoid of immune microenvironment. The present disclosure provides a paradigm shift in the understanding of patient tumours, which has a secretome that is clearly absent from in-vitro testing of studies in the art. The present disclosure provides a new understanding that cancer cells are not fully reliant on nutrients for survival and that cancer cells can evade cell death (e.g., shown by flow cytometry data) using the secretome.

In some examples, the agent may comprise but is not limited to a stimulating agent, a suppressing agent, and the like.

In some examples, the stimulating agent may include but is not limited to, vitamin D, IL-2, paramethoxyamphetamine (PMA) and/or IL-4, LPS (lipopolysaccharide), and the like.

In some examples, the suppressing agent may include an antibody, a receptor and/or a CD11b inhibitory drug (that binds and/or blocks CD11b). In some examples, the suppressing agent may include but is not limited to, GB1275, and the like.

In some examples, the agent is a stimulating agent.

In some examples, the agent is a myeloid stimulating agent.

In some examples, the agent is PMA and/or IL-4.

In some examples, the stimulating agent is PMA and IL-4.

In some examples, the PMA is used at concentrations such as from about 5 ng/ml to about 300 ng/ml, from 15 ng/ml to about 250 ng/ml, from 20 ng/ml to about 200ng/ml, from 25 ng/ml to about 150 ng/ml, from 30 ng/ml to about 100 ng/ml, from 35 ng/ml to about 50 ng/ml, 40 ng/ml to about 45 ng/ml, and the like.

In some examples, the PMA is used at concentrations such as 0.01 ng/ml, 0.02 ng/ml, 0.03 ng/ml, 0.04 ng/ml, 0.05 ng/ml, 0.06 ng/ml, 0.07 ng/ml, 0.08 ng/ml, 0.09 ng/ml, 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml, 1 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, 2 ng/ml, 2.1 ng/ml, 2.2 ng/ml, 2.3 ng/ml, 2.4 ng/ml, 2.5 ng/ml, 2.6 ng/ml, 2.7 ng/ml, 2.8 ng/ml, 2.9 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 30 ng/ml, 40 ng/ml, 50ng/ml, 60 ng/ml, 70 ng/ml, 80 ng/ml, 90 ng/ml, 100 ng/ml, 150 ng/ml, 200 ng/ml, 250 ng/ml, 300 ng/ml, and the like.

In some examples, the IL-4 is used at concentrations such as from about 5 ng/ml to about 200 ng/ml, from 15 ng/ml to about 150 ng/ml, from 20 ng/ml to about 100ng/ml, from 25 ng/ml to about 50 ng/ml, from 30 ng/ml to about 40 ng/ml, and the like.

In some examples, the IL-4 is used at concentrations such as 0.01 ng/ml, 0.02 ng/ml, 0.03 ng/ml, 0.04 ng/ml, 0.05 ng/ml, 0.06 ng/ml, 0.07 ng/ml, 0.08 ng/ml, 0.09 ng/ml, 0.1 ng/ml, 0.2 ng/ml, 0.3 ng/ml, 0.4 ng/ml, 0.5 ng/ml, 0.6 ng/ml, 0.7 ng/ml, 0.8 ng/ml, 0.9 ng/ml, 1 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, 2 ng/ml, 2.1 ng/ml, 2.2 ng/ml, 2.3 ng/ml, 2.4 ng/ml, 2.5 ng/ml, 2.6 ng/ml, 2.7 ng/ml, 2.8 ng/ml, 2.9 ng/ml, 3 ng/ml, 4 ng/ml, 5 ng/ml, 6 ng/ml, 7 ng/ml, 8 ng/ml, 9 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 80 ng/ml, 90 ng/ml, 100 ng/ml, 150 ng/ml, 200 ng/ml, and the like.

In some examples, the agent to generate the immune cell is incubated with the cell for a time such as but is not limited to 1 hour, 2 hours, 3 hours, 4 hours, 5 hours, 6 hours, 12 hours, 24 hours, 36 hours, 48 hours, 72 hours, 92 hours, and the like. In some examples, the agent to generate the immune cell is incubated with the cell for 48 hours.

In some examples, the agent to generate the immune cell is incubated with the cell for a time such as but is not limited to no more than 1 hour, no more than 2 hours, no more than 3 hours, no more than 4 hours, no more than 5 hours, no more than 6 hours, no more than 12 hours, no more than 24 hours, no more than 36 hours, no more than 48 hours, no more than 72 hours, no more than 92 hours, and the like. In some examples, the agent to generate the immune cell is incubated with the cell for no more than 48 hours.

In some examples, wherein after incubation with an agent the immune cell is washed and incubated alone or with a disease cell (such as a tumour cell)

In some examples, the stimulating agent generates the macrophage population to comprise at least 50%, or at least 60%, or at least 70%, or at least 80%, or at least 90%, or at least 91%, 92%, 93%, 94%, 95%, 96%, 97%, 98%, 99%, or substantially all cells express CD11b.

In some examples, the stimulating agent generates a population that comprise at least 50%, 55%, 60%, 65%, 70%, 80%, 81%, 82%, 83%, 84%, 85%, 86%, 87%, 88%, 89%, 90%, 91%, 92%, 93%, 94%, 95%, 96%, 97%, 98%, 99%, or CD11 b expressing cells. In some examples, the stimulating agent generates a population that comprises at least 80%, 81%, 82%, 83%, 84%, 85%, 86%, 87%, 88%, 89%, 90%, 91%, 92%, 93%, 94%, 95%, 96%, 97%, 98%, 99%, or CD11 b expressing cells.

In some examples, the secretome sustains cell growth, cell proliferation cryopreserve cells, elongates telomere length in cancer cells, involved in metabolic reprogramming (such as methylhistidine metabolism, biotin metabolism, taurine and hypotaurin metabolism), identifying drug resistance and/or preventing cancer cell death under nutrient stress conditions.

The inventor of the present disclosure showed that the secretome generated in the present disclosure is superior to standard growth media (serum-based or serum free) in sustaining cancer cell growth or proliferation and preventing cancer cell death under nutrient stress/conditions without nutrients.

In another aspect, there is provided a method of generating a macrophage-derived secretome comprising culturing an immune progenitor cell in the presence of a stimulating agent to thereby generate an activated immune cell population and harvesting the secretome from the generated activated immune cell population.

In some examples, there is provided a method of generating a composition as described in the present disclosure, comprising culturing a cell in the presence of an agent to generate immune cell, and harvesting the secretome generated by the immune cell.

In another aspect, there is provided a method of generating a composition, comprising culturing a cell in the presence of an agent to generate immune cell, and harvesting the secretome generated by the immune cell.

In some examples, the secretome is obtained by culturing a cell in the presence of an agent to generate an immune cell and harvesting the secretome generated by the immune cell.

In some examples, the secretome is obtained by culturing a cell in the presence of a stimulating agent to generate a CD11b expressing immune cell and harvesting the secretome generated by the immune cell.

In some examples, the secretome is obtained by culturing a myeloid cell line in the presence of a stimulating agent to generate a CD11b expressing macrophage and harvesting the secretome generated by the immune cell.

In some examples, the secretome is obtained by culturing U937 cells in the presence of PMA+IL-4 mixture to generate CD11b expressing macrophages and harvesting the secretome generated by the CD11b expressing macrophages.

In some examples, the secretome is obtained by culturing U937 cells in the presence of PMA+IL-4 mixture to generate CD11b expressing macrophages and harvesting the secretome generated by the macrophages.

In some examples, the secretome comprises CCL2/MCP-1, MIP-1α/MIP-1β,CCL5/RANTES, MIF, IL-1ra/IL-IF3, and/or IL-8.

In some examples, the secretome comprises CCL2/MCP-1, MIP-1α/MIP-1β,CCL5/RANTES, MIF, IL-1ra/IL-IF3, IL-8 and/or Serpin-E1/PAI-1.

In some examples, the secretome is extracted/harvested by extraction methods such as but is not limited to ultrafiltration, precipitation, dialysis, and the like.

In some examples, the secretome is extracted/harvested by removing cells or fragments thereof from the media.

In some examples, the secretome can be produced/harvested in 1 hour, 2 hours, 3 hours, 4 hours, 5 hours, 6 hours, 7 hours, 8 hours, 9 hours, 10 hours, 11 hours, 12 hours, 18 hours, 24 hours, 30 hours, 36 hours, 42 hours, 48 hours and the like. In some examples, the secretome is produced/harvested in 48 hours. In some examples, the secretome is produced/harvested in 3 hours. In some examples, the secretome is produced/harvested in 24 hours. In some examples, the secretome is produced/harvested in 48 hours.

In some examples, the secretome is harvested from CD11b expressing cells after incubation for at least 3 hours, at least 6 hours, at least 12 hours, at least 24 hours, at least 36 hours, at least 48 hours, at least 72 hours, and the like.

In some examples, the secretome is harvested from CD11b expressing cells after incubation for at least 3 hours.

In some examples, the secretome is harvested from CD11b expressing cells after incubation for at least 24 hours. In some examples, secretome after incubation of CD 11b expressing cells with agent and harvested in at least 24 hours provides a higher boost potential (such as higher tumour cell proliferation) to the secretome harvested as compared to the secretome harvested at 3 hours.

In some examples, the secretome is harvested from CD11b expressing cells after incubation for at least 48 hours. In some examples, secretome after incubation of CD 11b expressing cells with agent and harvested in at least 48 hours provides a higher boost potential (such as higher tumour cell proliferation) to the secretome harvested as compared to the secretome harvested at 3 hours.

In some examples, the secretome is harvested from CD11b expressing cells after incubation for about 3 hours, about 6 hours, about 12 hours, about 24 hours, about 36 hours, about 48 hours, about 72 hours, and the like.

In some examples, the secretome is harvested from CD11b expressing cells after incubation for about 3 hours.

In some examples, the secretome is harvested from CD11b expressing cells after incubation for about 24 hours.

In some examples, the secretome is harvested from CD11b expressing cells after incubation for about 48 hours.

In some examples, the secretome is harvested from CD11b expressing cells after incubation for no more than 3 hours, no more than 6 hours, no more than 12 hours, no more than 24 hours, no more than 36 hours, no more than 48 hours, no more than 72 hours, and the like.

In some examples, the secretome is harvested from CD11b expressing cells after incubation for no more than 3 hours.

In some examples, the secretome is harvested from CD11b expressing cells after incubation for no more than 24 hours.

In some examples, the secretome is harvested from CD11b expressing cells after incubation for no more than 48 hours.

In some examples, the secretome is harvested from CD11b expressing cells after incubation for at least 1 hour to at least 48 hours. In some examples, the secretome harvested from CD 11b expressing cells after incubation for at least 1 hour to at least 24 hours.

In some examples, the secretome is stable in storage temperature such as but is not limited to, 4° C. 0° C., −20°C., −80°C., and the like. In some examples, the secretome is stable in −80° C.

In some examples, the secretome is stable for 1 month, 3 months, 6 months, 9 months, 1 year, 1.5 years, 2 years, 2.5 years, 3 years, 3.5 years, 4 years, 5 years, 6 years, 7 years, 8 years, 9 years, 10 years, and the like. In some examples, the secretome is stable for 3 years. In some examples, the secretome is stable in −80° C. for 3 years.

As used herein, the term “stable” refers to a fixed and steady condition that is reliable. A stable item/product is typically resistant to fluctuation in conditions, thermally or physiologically. Therefore, a stable item/product is understood to be resistant to thermal changes that can lead to degradation or denaturation.

In another aspect, there is provided a method of generating a macrophage-derived secretome comprising culturing a cell in the presence of a stimulating agent to thereby generate a macrophage population and harvesting the secretome from the generated macrophage.

In some examples, the macrophage population is a population where at least 1%, at least 10%, at least 20%, at least 30%, at least 40%, at least 50%, or at least 60%, or at least 70%, or at least 80%, or at least 90%, or at least 91%, 92%, 93%, 94%, 95%, 96%, 97%, 98%, 99%, or substantially all cells express CD11 b.

The inventor of the present disclosure has developed a method of generating a macrophage-derived secretome. This secretome-based product sustains cancer cells during: (1) Nutrient stress (2) Drug response.

Advantageously, the inventor of the present disclosure provides a method that is scalable commercially. The secretome of the present disclosure can be harvested in large quantities upon culturing an immune progenitor cell in the presence of an agent for 48 hours, packaged in cryovials and is stable in −80° C. storage for 3 years or more. Old product stocks that the inventor of the present disclosure stored from 3 years ago are equally effective as a freshly made batch. It can be shipped out on dry ice to consumers/international partners. Laboratory testing can be provided to pharmaceutical companies to test their drug libraries/to test effects of microenvironment factors on efficacy of their new drugs before their drugs enter clinical trials among patients.

In some examples, the method of the present disclosure comprises culturing the cell in the presence of a diseased cell.

In some examples, the method of the present disclosure comprises culturing the activate immune cell in the presence of a diseased cell.

As used herein, the term “diseased cell” refers to a cell that is structurally or physiologically not healthy. A diseased cell may be abnormal, corrupt, or aberrantly affected by a disease.

In some examples, the diseased cell as described herein may comprise a cell from a proliferative disease.

The proliferative disease as described herein includes tumour and/or inflammatory disease. The proliferative cell as described herein includes cell from a proliferative disease.

In some examples, the tumour may be a benign tumour. In some examples, the tumour may be a malignant tumour or cancer. In some examples, the disease is a cancer including, but not limited to, breast cancer, lung cancer, colorectal cancer, and the like. In some examples, the disease is cancer such as, but not limited to, brain tumour, medulloblastoma, embryonal brain tumour (atypical teratoid rhabdoid tumour (ATRT)), germ cell tumour, and the like. In some examples, the cancer includes tumour subclasses and molecular/genomic subtypes that tend to relapse such as, but is not limited to, solid cancers, liquid cancers, and the like. In some examples, solid cancers that tend to relapse may include but is not limited to, atypical teratoid rhabdoid tumour (ATRT), medulloblastoma, and the like. In some examples, liquid cancers that tend to relapse may include but is not limited to, leukaemia, lymphoma, and the like.

In some examples, the proliferative disease may be inflammatory disease such as an acute inflammatory disease and/or a chronic inflammatory disease. In some examples, chronic inflammation/chronic inflammatory disease acute inflammation/acute inflammatory disease may include but is not limited to ulcerative colitis, Crohn's disease, infectious disease, and the like.

In some examples, the infectious disease may be caused by a bacterial pathogen, a viral pathogen, a fungal pathogen, or a parasite.

Examples of a bacterial pathogen may include, but is not limited to, Escherichia coli, Mycobacteria spp, Salmonella spp, Staphylococcus spp, Clostridium difficile, Listeria monocytogenes, Group B Streptococci, vancomycin-resistant Enterococci (VRE), and the like. Examples of a viral pathogen may include, but is not limited to, Human papillomavirus, Rhinovirus, Human cytomegalovirus in HIV-1 positive patient, Hepatitis virus, Coronavirus (CoV), severe acute respiratory syndrome (SARS), monkey pox virus and the like.

Examples of a fungal pathogen may include, but is not limited to, Botrytis cinerea, Pseudomonas syringae, Fusarium oxysporum and the like.

Examples of a parasite may include, but is not limited to, Leishmania parasites, Giardia, Cryptosporidum, Entamoeba and the like.

In some examples, the inflammatory disease may be a neurodegenerative disease, such as neuroinflammation that leads to neurodegeneration/brain damage/nerve damage. In some examples, the disease may be a neurodegenerative disease such as dementia, Alzheimer's disease, Parkinson's, Multiple Sclerosis, and the like. In some examples, the inflammatory disease may include but is not limited to fibrosis, muscle aging, and the like.

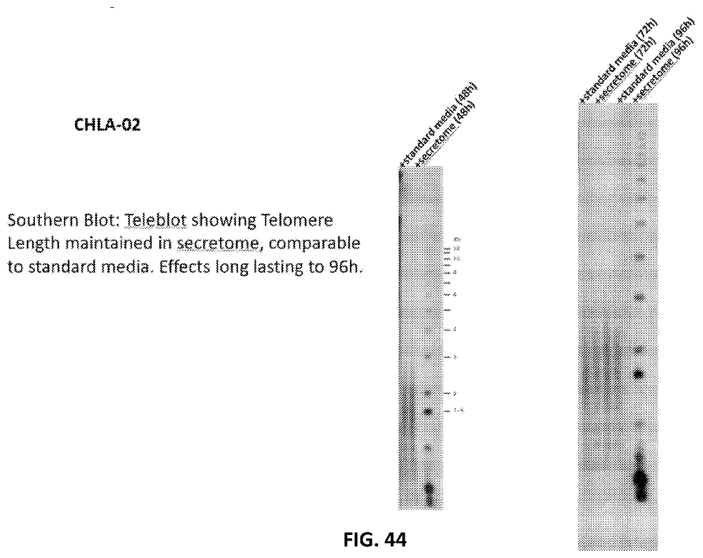

Without wishing to be bound by theory, telomere length is known in the art to affect longevity of non-diseased cells. In non-diseased cells, if telomere can be elongated, this will provide anti-aging properties/prolong the lifespan of the cell. As shown by the experimental data of the present disclosure (FIG. 44), telomere length was tested ATRT cancer cells. Telomere length (involved in aging) of ATRT cancer cells is maintained and elongated up to 96 hours in secretome. Telomere length is elongated at time points 48 to 72 hours and significantly elongated at 96 hours. Hence, the secretome could be further implicated in anti-aging properties which will have a broad impact on many diseases in health and medicine/in anti-aging drug development.

In some examples, the diseased cell is brain tumour.

In some examples, the diseased cell is atypical teratoid rhabdoid tumour (ATRT).

In some examples, the diseased cell is medulloblastoma.

In some examples, the diseased cell is a brain tumour, an atypical teratoid rhabdoid tumour (ATRT) and/or medulloblastoma.

In some examples, the ATRT includes molecular subtypes of such as but is not limited to, MYC-ATRT, tyrosinase-ATRT (TYRATRT), sonic hedgehog-ATRT (SHH-ATRT), and the like.

The inventor of the present disclosure uncovered specific molecular subtypes of ATRT that were secretome enriched, matching macrophage-enrichment patterns (MYCATRTS>TYR-ATRTs>SHH-ATRTs).

In some examples, the diseased cell may include but is not limited to cancer cell lines and/or non-cancer cell lines.

In some examples, the number of diseased cell that are cultured/seeded with an activated immune cell may include at least 50 cells, at least 100 cells, at least 150 cells, at least 200 cells, at least 250 cells, at least 300 cells, at least 350 cells, at least 400 cells, at least 450 cells, at least 500 cells, at least 550 cells, at least 1000 cells, at least 1500 cells, at least 2000 cells, at least 2500 cells, at least 3000 cells, at least 3500 cells, at least 4000 cells, at least 4500 cells, at least 5000 cells, at least 5500 cells, at least 6000 cells, at least 6500 cells, at least 7000 cells, at least 7500 cells, at least 8000 cells, at least 8500 cells, at least 9000 cells, at least 9500 cells, at least 10000 cells, and the like.

In some examples, the number of diseased cell that are cultured/seeded with an activated immune cell may include about 50 cells, about 100 cells, about 150 cells, about 200 cells, about 250 cells, about 300 cells, about 350 cells, about 400 cells, about 450 cells, about 500 cells, about 550 cells, about 1000 cells, about 1500 cells, about 2000 cells, about 2500 cells, about 3000 cells, about 3500 cells, about 4000 cells, about 4500 cells, about 5000 cells, about 5500 cells, about 6000 cells, about 6500 cells, about 7000 cells, about 7500 cells, about 8000 cells, about 8500 cells, about 9000 cells, about 9500 cells, about 10000 cells, and the like.

In some examples, diseased cell may be cultured with an activated immune cell in a petri-dish, 6 well plate, a 12 well plate, a 24 well plate, a 96 well plate, a T-25 flask, a T-75 flask, and the like.

In some examples, cancer cell lines may include but is not limited to ATRT cell lines, medulloblastoma cell lines, and the like.

In some examples, the ATRT cell lines may include but is not limited to CHLA-04, CHLA-05, CHLA-02, BT-12, CHLA-06, BT-37, ATRT95, CHLA-266, and the like.

In some examples, the medulloblastoma cell lines may include, but is not limited to, Daoy, D341, CHLA-01, CHLA-01R, and the like.

In some examples, the medulloblastoma cell lines may include resistant medulloblastoma cell lines such as but is not limited to CHLA-01R, and the like.

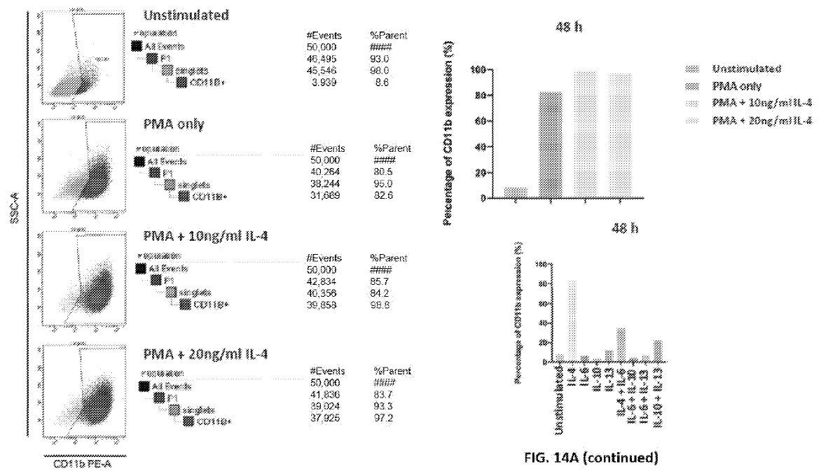

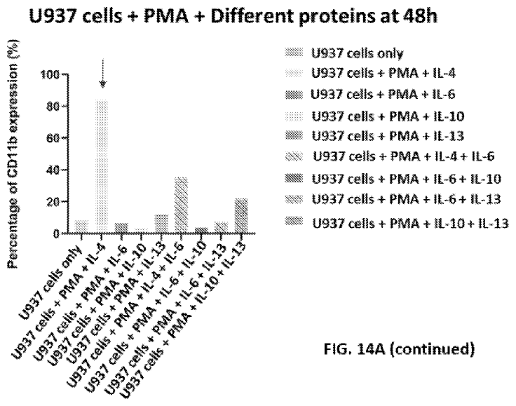

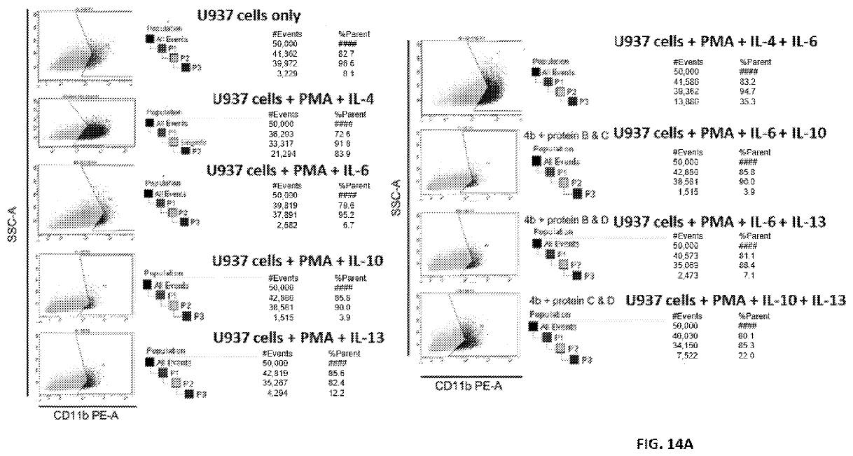

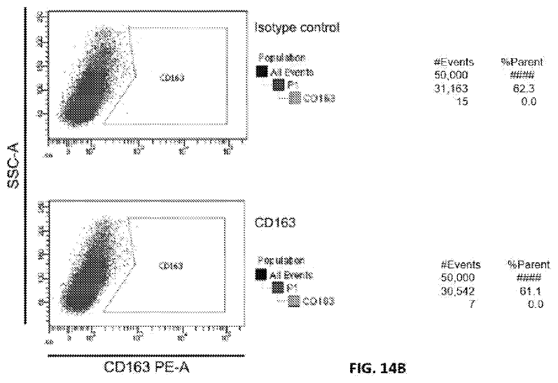

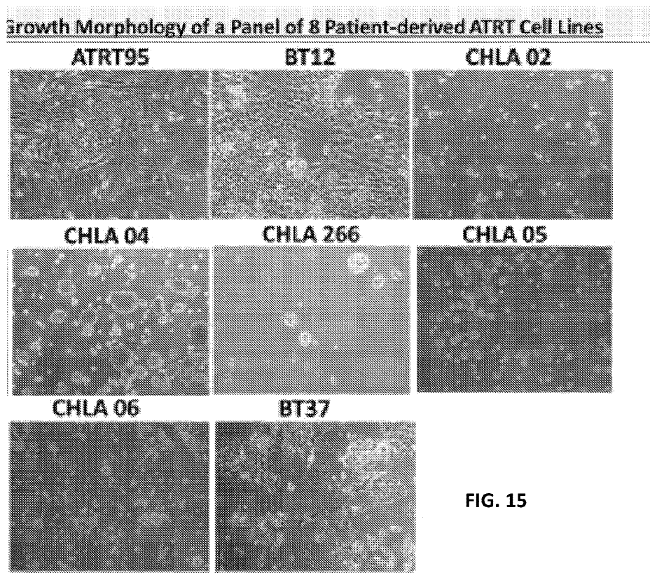

As shown in FIG. 15, CHLA-06 was the most aggressive and fastest growing in-vitro phenotype in the panel of ATRT tumour models of the present disclosure. ATRT95 was too slow growing and not suitable for in-vitro experiments. The inventor of the present disclosure selected CHLA-06, the most aggressive ATRT tumour cells from the panel of 7 ATRT cell line of the present disclosure to create the tumour compartment, based on the assumption that the most rapid growing in-vitro cell line will provide/secrete the most complete spectrum/concoction of tumour-promoting factors.

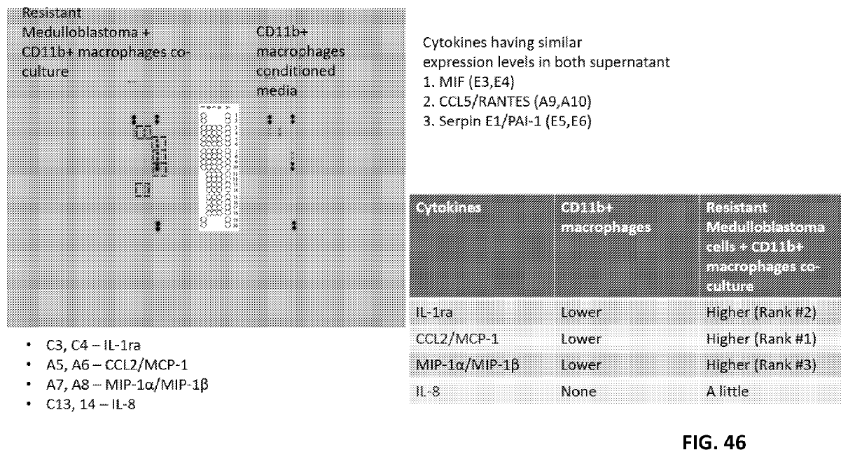

As shown in FIG. 46, the composition generated from immune cells (such as CD11b+ cells) in the presence of resistant medulloblastoma tumour cells led to an increase in expression levels of CCL2/MCP-1, IL-1ra, MIP-1a/MIP-18 and IL-8 (from highest to lowest expression) as compared to without the diseased cell. There was no increase in expression levels in MIF, CCL5/RANTES, Serpin E1/PAI-1 between the two conditions (i.e., presence vs absence of resistant medulloblastoma cell line).

In some examples, non-cancer cell lines may include but is not limited to skin cells, nerve cells, brain cells, liver, heart, and the like.

In the experimental data of the present disclosure, expression of markers are compared between patient tumours, ATRT cell lines, foetal and childhood normal brain controls. In patient tumours, as the inventor of the present disclosure have shown in the Cancer Model (ATRT and other types of brain tumours) of the present disclosure that the secretome play an important role in tumour microenvironment among patients and affects drug efficacy in-vitro.

Without wishing to be bound by theory, cancer relapse is an area of intense research in current oncology fields, it is not well understood how macrophages mediate cancer relapse. Paediatric brain cancer is the leading cause of death in childhood cancer. ATRT is a paediatric embryonal brain tumour, frequently occurring in infants and young children, accounting for 15-20% of all brain tumours in children less than 3 years of age. It is an aggressive brain tumour and historical survival outcome is poor (10-20%, 5-year survival for patients<3 years old). Standard of care historically involved surgery, chemotherapy and radiation therapy, although none are curative therapies. A recently published trial ACNS0333 employing high dose chemotherapy and peripheral blood stem cell rescue has improved survival. However, this improved survival was achieved at the cost of treatment toxicity with high dose chemotherapy and the subsequent requirement for peripheral blood stem cell rescue. High rates of treatment failure were also observed in this trial. More novel effective drugs and a deeper understanding on the tumour microenvironment in ATRTs impacting drug efficacy and tumour biology are needed.

The inventor of the present disclosure discovered that macrophage-derived secretome is found in the high relapse risk subtype of ATRT (an aggressive childhood brain tumour). As cancer cells are able to survive and proliferate in the presence of the secretome under nutrient stress conditions/without any nutrients, this provides a mechanism for cancer cells to survive until new nutrients become available and then proliferate rapidly. This therefore provides an advantageous mechanism for relapse. The inventor of the present disclosure discovered a six-factor-secretome from macrophages, remarkably sustains a critical cell mass during nutrient stress in a paediatric embryonal brain tumour, atypical teratoid rhabdoid tumour (ATRT). Specific ATRT-subtypes emerged as secretome-enriched, matching macrophage-enrichment patterns and were high-relapse-risk subtypes. Secretome alters drug response, protects against cell death and provides pro-survival niches to rescue drugged cells. ATRT cells rearrange to form a weblike architecture in secretome that is stable during drug exposure, suggesting a mechanism for therapy resistance. Medulloblastoma cells form the same stringy chain patterning as ATRT cells when grown in secretome from 4 hours up to 96 hours. Strikingly, secretome prevents tumour cell death for prolonged periods in aggressive tumour models and models of cerebrospinal dissemination, suggesting a role in tumour resistance and relapse. The results of the present disclosure unravel for the first time, a previously unexplored role of a unique macrophage-secretome, providing an alternative fuel to sustain cancer cells during nutrient stress, and implications in relapse subtypes. This secretome-product can be applied to other cancer types because the inventor of the present disclosure has found these six-factors secretome enrichment among many other adult cancer types. In addition, the secretome-product can be applied to other non-cancer human disease such as chronic inflammation, ulcerative colitis, Crohn's disease, Alzheimer's disease/dementia, Coronavirus disease, human immunodeficiency virus (HIV) which may share this secretome. Hence, the implications and applications are broad.

The secretome can be utilized as a cell culture media and in cell studies for biomarker discovery. Furthermore, the secretome can also be utilized in therapeutic applications such as but is not limited to, tissue regeneration (such as skin regeneration/skin graft manufacturing, nerve/brain regeneration and/or degeneration, vascular regeneration/blood vessel regeneration, liver regeneration, regeneration of tissues/organs for transplant, heart regeneration, corneal regeneration (such as corneal graft synthesis), chondrocyte regeneration, and the like), wound healing, angiogenesis, osteogenesis, treatment of inflammatory response, cryopreservation of cells, and/or drug discovery (such as personalized drug discovery). The secretome can also be utilized as a cell culture media and in cell studies for biomarker discovery, as a drug/compound to elongate telomere in cells to reverse the process of aging in human cells (such as diseased and/or non-diseased cells)

In some examples, the secretome of the present disclosure can be used for:
i) therapeutics such as for drug screening, measuring effectiveness of drugs in secretome rich tumours and/or
ii) diagnostics such as developing kits/assays for diagnosis of secretome rich tumours.

In some examples, the kit for diagnosis of secretome rich tumours may include the secretome factors as disclosed herein.

In another aspect, there is provided a composition for use in tissue regeneration.

In some examples, there is provided a method of regenerating a tissue. In some examples, the tissue may include but is not limited to skin, chondrocyte/cartilage, corneal, liver, heart, nerve, blood vessel, and the like.

In some examples, there is provided a method of enhancing or initiating skin regeneration comprising administering the secretome as described herein to a subject in need thereof or a cell from a subject in need thereof. In some examples, the subject in need of skin regeneration may include but is not limited to burn patients, and the like. In some examples, the method may be a dermatological or aesthetic method. In some examples, the method of skin regeneration may include efficient generation of skin grafts using the secretome of the present disclosure.

In some examples, the secretome of the present disclosure may be used for skin regeneration. In some examples, skin regeneration may include efficient generation of skin grafts using the secretome of the present disclosure. In some examples, skin regeneration may be used for dermatological/aesthetic applications. In some examples, skin regeneration may be used for, such as, but is not limited to burn patients, and the like. In some examples, skin regeneration may include skin grafts for keloid scar surgery.

In some examples, there is provided a method of enhancing or initiating nerve/brain regeneration/anti-aging comprising administering the secretome as described herein to a subject in need thereof or a cell from a subject in need thereof. In some examples, the subject in need of nerve/brain regeneration/anti-aging may include but is not limited to patients with stroke, Alzheimer's disease, vascular dementia, Lewy body disease, and the like.

In some examples, the secretome of the present disclosure may be used for nerve/brain regeneration/anti-aging. In some examples, nerve/brain regeneration may be used for, such as, but is not limited to, stroke, Alzheimer's disease, vascular dementia, Lewy body disease, and the like.

In some examples, there is provided a method of enhancing or initiating spinal cord regeneration comprising administering the secretome as described herein to a subject in need thereof or a cell from a subject in need thereof. In some examples, the subject in need of spinal cord regeneration may include but is not limited to patients with severed nerves/spinal cord, and the like.

In some examples, the secretome of the present disclosure may be used for spinal cord regeneration. In some examples, spinal cord regeneration may be used for, such as, but is not limited to, patients with severed nerves/spinal cord, and the like.

In some examples, there is provided a method of enhancing or initiating vascular regeneration/blood vessel regeneration comprising administering the secretome as described herein to a subject in need thereof or a cell from a subject in need thereof. In some examples, the subject in need of vascular regeneration may include but is not limited to, patients with clogged/dysfunctional blood vessels (such as heart blood vessels, leg blood vessels), patients with diabetes, and the like.

In some examples, the secretome of the present disclosure may be used for vascular regeneration. In some examples, vascular regeneration may be used for, such as, but is not limited to patients with clogged/dysfunctional blood vessels (such as heart blood vessels, leg blood vessels), patient with diabetes, patient with coronary heart diseases (e.g., patients with impending heart attack due to clogged up blood vessels), and the like. In some examples, vascular regeneration may be performed using stem cells (such as induced pluripotent stem cells (iPSCs)) of endothelial cells).

In some examples, there is provided a method of enhancing or initiating heart regeneration comprising administering the secretome as described herein to a subject in need thereof or a cell from a subject in need thereof. In some examples, the subject in need of heart regeneration may include but is not limited to patients who require heart transplant, and the like.

In some examples, the secretome of the present disclosure may be used for heart regeneration. In some examples, heart regeneration may be used for, such as, but is not limited to, patients who require heart transplant, and the like.

In some examples, there is provided a method of enhancing or initiating liver regeneration comprising administering the secretome as described herein to a subject in need thereof or a cell from a subject in need thereof. In some examples, the subject in need of liver regeneration may include but is not limited to patients who require liver transplant, and the like.

In some examples, the secretome of the present disclosure may be used for liver regeneration. In some examples, liver regeneration may be used for, such as, but is not limited to, patients who require liver transplant, and the like. In some examples, liver regeneration may be performed with hepatocytes and/or liver cancer cells. In some examples, there is provided a method of enhancing or initiating cryopreservation of cells comprising administering the secretome as described herein to a subject in need thereof or a cell from a subject in need thereof.

In some examples, there is provided a method of enhancing or initiating cryopreservation of cancer cells and/or healthy cells comprising administering the secretome as described herein to a subject in need thereof.

In some examples, the secretome of the present disclosure may be used for cryopreservation of cells. In some examples, the secretome of the present disclosure may be used for cryopreservation of cancer cells and/or healthy cells. In some examples, healthy cells that may be cryopreserved with the secretome may include but is not limited to, eggs, sperm, cord blood, and the like. This may be useful for fertility preservation of cells such as but is not limited to eggs, sperm, and the like.

Without wishing to be bound by theory, studies in the art cryopreserve cells in standard media with dimethyl sulfoxide (DMSO) to maintain cell viability. The secretome of the present disclosure may be used in cryopreserving cells and may be compared with the performance of using standard media. In some examples, there is provided a method of drug discovery comprising contacting the secretome as described herein to an agent of interest. In some examples, there is provided a method of testing the effects of drugs/anti-viral agents against diseases in the presence of the secretome.

In some examples, the secretome of the present disclosure may be used in drug discovery. In some examples, the secretome of the present disclosure may be used to test the effects of drugs/anti-viral agents against diseases in the presence of the secretome. In some examples, the diseases may include but is not limited to coronavirus disease (COVID-19), human immunodeficiency virus (HIV), and the like.

In some examples, there is provided a method of enhancing or initiating chondrocyte/cartilage regeneration comprising administering the secretome as described herein to a subject in need thereof. In some examples, the subject in need of chondrocyte/cartilage regeneration may include but is not limited to patients with cartilage damage and requiring cartilage repair, patients with cartilage degeneration in knees, hips, spine, osteoarthritis/old age, and the like.

In some examples, the secretome of the present disclosure may be used for chondrocyte regeneration. In some examples, chondrocyte regeneration may be used for, such as, but is not limited to patients with cartilage damage, and the like.

In some examples, there is provided a method of enhancing or initiating corneal regeneration/corneal graft synthesis comprising administering the secretome as described herein to a subject in need thereof. In some examples, the subject in need of corneal regeneration/corneal graft synthesis may include but is not limited to patients with corneal damage/injuries and requiring corneal repair, and the like.

In some examples, the secretome of the present disclosure may be used for corneal regeneration/corneal graft synthesis. In some examples, corneal regeneration may be used for, such as, but is not limited to patients with corneal damage/injuries, and the like.

In another aspect, there is provided a method of testing/screening a drug/inhibitor/compound comprising culturing the drug with a diseased cell in the presence of the composition of the present disclosure.

In some examples, the drug/inhibitor/compound may comprise but is not limited to, drugs (such as oncology drugs, anti-inflammatory drugs, and the like), compounds (including but not limited to naturally found compounds, synthetic compounds, and the like), small molecules, antigen binding proteins (such as antibodies), and the like.

In some examples, the drug/inhibitor/compound works if the diseased cell dies; or wherein the drug/inhibitor/compound does not work if the diseased cell thrives.

The inventor of the present disclosure showed that drug response is dampened in the presence of the secretome of the present disclosure. In detail, the secretome of the present disclosure enriched in high-risk tumours such as paediatric brain tumour (ATRT) and the presence of secretome with cancer cells in vitro decreases the efficacy of effective cancer drugs. Therefore, a drug that is effective in standard media during in vitro-testing may not be equally efficacious in patient tumours which harbour immune rich conditions.

The secretome product of the present disclosure provides immune rich conditions for laboratory testing of diseased cells (such as any type of cancer cells), allowing testing of new/novel drugs on cancer cells or screening of compounds to identify drugs effective against diseased cells/cancer cells in an immune-rich in-vitro cultures, by mimicking microenvironment conditions in patient tumours. It enables the inventor of the present disclosure to identify cancer cells which may be more responsive to immune microenvironment modulation. It also allows the inventor of the present disclosure to evaluate if new drugs may be potentially more effective in immune-rich patient tumours and identify drugs that will fail in certain tumours (immune rich) before the drugs are employed in Phase I trials on patients. Clinical trials are costly, failed clinical trials will escalate the cost of drug development. This is a critical step—efficacy of drugs in immune-rich conditions is less well-understood—which may explain why some drugs found effective in the laboratory, fail when they are administered to patients during clinical trials. This secretome-product will provide a drug development tool to enable testing of new drugs/drug libraries in-vitro for high throughput drug discovery, under immune rich conditions of cancer cells. This can be done in a laboratory setting prior to application in patients during a clinical trial and is useful for pharmaceutical industries. This secretome also allows screening of patient tumours which are more likely to respond to certain drugs and select suitable patients with immune rich tumours for clinical trials.

In some examples, the method of the present disclosure comprises customizing a drug/inhibitor/compound based on the markers that are expressed in a diseased cell.

In some examples, the marker that is expressed in ATRT cells may include cell cycle markers/cell cycle inhibitors. In some examples, the markers that are expressed in ATRT cells may include markers that are found upregulated in cancer compared to normal tissue. In some examples, the marker that is expressed in ATRT cells may include but is not limited to maternal embryonic leucine kinase (MELK), kinesin family member 11 (KIF11), and the like. In some examples, the marker that is expressed in ATRT cells is maternal embryonic leucine kinase (MELK).

In some examples, the drug/inhibitor/compound is a MELK inhibitor. In some examples, the MELK inhibitor is 1-(6-(3,5-dichloro-4-hydroxyphenyl)-4-(((1r,4r)-4-((dimethylamino)methyl)cyclohexyl)amino)-1,5-naphthyridin-3-yl)ethenone (OTSSP167). In some examples, the drug/inhibitor/compound is a Kif11 inhibitor. In some examples, the Kif11 inhibitor is Ispinesib.

In some examples, OTSSP 167 is used at concentrations such as from about 0.01 nM to about 600 nM, from about 0.05 nM to about 550 nM, from about 0.1 nM to about 500 nM, from about 0.15 nM to about 450 nM, from about 0.2 nM to about 400 nM, from about 0.25 nM to about 350 nM, from about 0.3 nM to about 300 nM, from about 0.35 nM to about 250 nM, 0.4 nM to about 200 nM, 0.45 nM to about 150 nM, 0.5 nM to about 100 nM, 1 nM to about 50 nM, 1.5 nM to about 40 nM, 2 nM to about 45 nM, 2.5 nM to about 40 nM, 3 nM to about 35 nM, 3.5 nM to about 30 nM, 4 nM to about 25 nM, 4.5 nM to about 20 nM, 5 nM to about 15 nM, 5.5 nM to about 10 nM, and the like.

In some examples, OTSSP 167 is used at concentrations such as 0.01 nM, 0.05 nM, 0.1 nM, 0.15 nM, 0.2 nM, 0.25 nM, 0.3 nM, 0.35 nM. 0.4 nM, 0.45 nM, 0.5 nM, 1 nM, 1.5 nM, 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 15 nM, 20 nM, 25 nM, 30 nM, 35 nM, 40 nM, 45 nM, 50 nM, 55 nM, 60 nM, 65 nM, 70 nM, 75 nM, 80 nM, 85 nM, 90 nM, 95 nM, 100 nM, 150 nM, 200 nM, 250 nM, 300 nM, 350 nM, 400 nM, 450 nM, 500 nM, 550 nM, 600 nM, and the like.

In some examples, Ispinesib is used at concentrations such as from about 0.01 nM to about 600 nM, from about 0.05 nM to about 550 nM, from about 0.1 nM to about 500 nM, from about 0.15 nM to about 450 nM, from about 0.2 nM to about 400 nM, from about 0.25 nM to about 350 nM, from about 0.3 nM to about 300 nM, from about 0.35 nM to about 250 nM, 0.4 nM to about 200 nM, 0.45 nM to about 150 nM, 0.5 nM to about 100 nM, 1 nM to about 50 nM, 1.5 nM to about 40 nM, 2 nM to about 45 nM, 2.5 nM to about 40 nM, 3 nM to about 35 nM, 3.5 nM to about 30 nM, 4 nM to about 25 nM, 4.5 nM to about 20 nM, 5 nM to about 15 nM, 5.5 nM to about 10 nM, and the like In some examples, Ispinesib is used at concentrations such as from about 0.01 nM to about 600 nM, from about 0.05 nM to about 550 nM, from about 0.1 nM to about 500 nM, from about 0. 15 nM to about 450 nM, from about 0.2 nM to about 400 nM, from about 0.25 nM to about 350 nM, from about 0.3 nM to about 300 nM, from about 0.35 nM to about 250 nM, 0.4 nM to about 200 nM, 0.45 nM to about 150 nM, 0.5 nM to about 100 nM, 1 nM to about 50 nM, 1.5 nM to about 40 nM, 2 nM to about 45 nM, 2.5 nM to about 40 nM, 3 nM to about 35 nM, 3.5 nM to about 30 nM, 4 nM to about 25 nM, 4.5 nM to about 20 nM, 5 nM to about 15 nM, 5.5 nM to about 10 nM, and the like

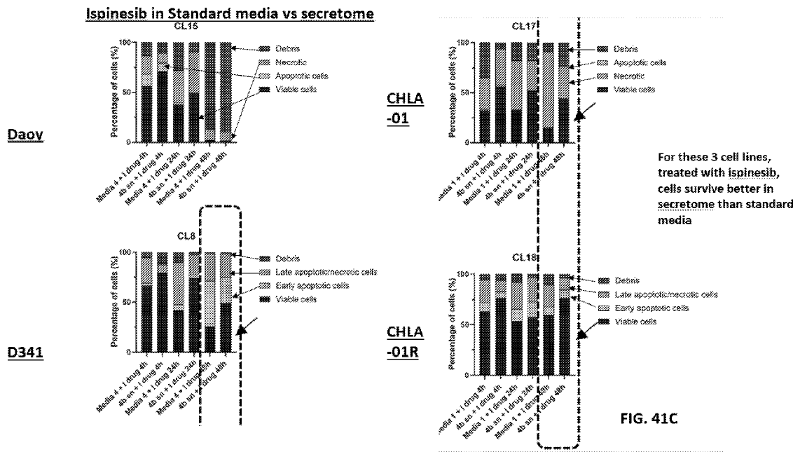

As shown in the experimental data of the present disclosure, six ATRT tumour cell lines were cultured in macrophage-derived secretome and compared against normal brain controls. Maternal embryonic leucine kinase (MELK) is found to be highly expressed across ATRT subtypes and expression low in normal brain samples. The inventor of the present disclosure showed that OTSSP167, a Phase I/II molecular inhibitor of MELK was efficacious across a panel of six ATRT cell lines and on an aggressive orthotopic xenograft mouse model. Presence of the secretome reduced drug efficacy of OTSSP167 during early tumourigenesis in ATRT cell lines and medulloblastoma. Phase I/II molecular inhibitor (R)-N-(3-aminopropyl)-N-(1-(3-benzyl-7-chloro-4-oxo-3,4-dihydroquinazolin-2-yl)-2-methylpropyl)-4-methylbenzamide (Ispinesib) was also tested in ATRT cell lines and medulloblastoma. In the presence of the secretome, the drug efficacy of Ispinesib was reduced during early tumourigenesis in ATRT cell lines and medulloblastoma. The secretome can keep medulloblastoma cells more viable compared to standard media in the absence of drugs. In the presence of OTSSP167, neurospheres (derived from primary and recurrent medulloblastoma from the same patient) were able to survive better in secretome compared to standard media. In the presence of Ispinesib, three medulloblastoma cell lines (also neurospheres) survive better in secretome compared to standard media. In some instances, as shown in FIG. 41 some of the medulloblastoma cell lines are more sensitive to drug treatment (e.g., OTSSP167 or ispinesib) in the presence of secretome as compared to standard media. Secretome protects tumour cells from drug killing in medulloblastoma cell lines that propagate as neurospheres (stem-like). The present disclosure highlights the gap of studies in the art which conducts in-vitro drug testing on cancer cell lines in standard media alone.

As used herein, the term “neurospheres” refer to a 3-dimensional, free-floating colony that forms in the presence of mitogens and consists of hundreds of cells of which a small percentage are stem cells, and the remainder are progenitors. Neurospheres may be derived from primary and recurrent diseased cells (such as medulloblastoma) from the same patient. Neurospheres are usually stem-cell enriched.

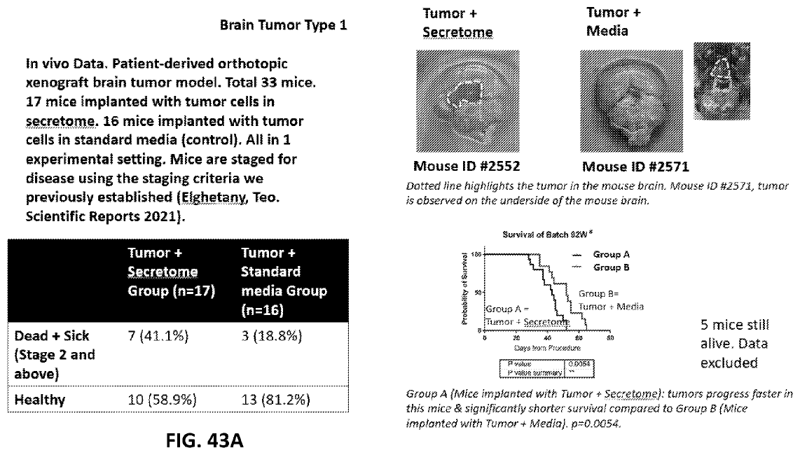

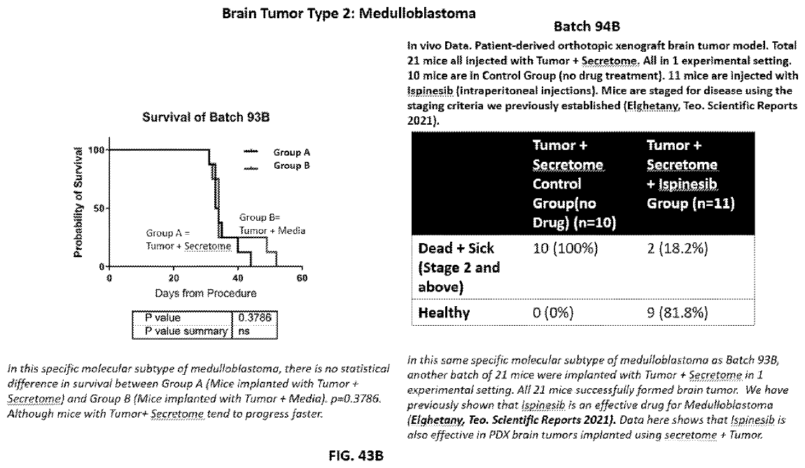

The effects of the secretome in promoting tumour progression are also shown in vivo in FIG. 43. The inventor of the present disclosure injected another brain tumour type (germ cell tumour which is a metastatic tumour) in the presence of secretome versus standard media (standard approach) into immunosuppressed mice. The mice injected with brain tumour in the presence of secretome started dying while the mice injected with brain tumour in standard media (no secretome) are still alive.

In some examples, the present disclosure studies the pathways that are altered by the secretome in a cancer cell microenvironment. In some examples, the methods to study the pathways that are altered by the secretome in a cancer cell microenvironment may include but is not limited to RNA-seq, metabolomics (such as semi-targeted metabolomics), protein-phospho array, antibody array assay (such as single sample, phosphor explorer array (PEX100)), complete antibody array assay (such as cytokine profiling antibody array (SCK100)), whole genome sequencing, whole exome sequencing, and the like.

In some examples, the present disclosure studies the effects of the secretome on metabolome changes in the tumour. In some examples, the methods to study the metabolome changes in the tumour may include but is not limited to metabolomics (such as semi-target metabolomics (full scan) untargeted orbitrap), and the like.

In some examples, the present disclosure studies the effects of the secretome on vascular changes in the tumour. In some examples, vascular changes in the tumour may be studied with in vivo models and/or in vitro models.

In some examples, in vivo models may include but is not limited to brain tumour models, non-brain tumour models, non-tumour brain models, patient-derived orthotopic xenograft model, and the like.

The present disclosure includes the harvesting of the brains of the in vivo models (such as mice), sectioning the brains (such as control sample, sample injected with tumour cells and standard media)), staining the blood vessels using methods such as immunohistochemistry, quantitating and comparing the blood vessels between secretome versus standard media.

In some examples, brain tumour models (such as brain tumour models) may include endothelial/blood vessel iPSCs co-cultured with brain tumour cells in secretome versus standard media.

As shown in FIG. 43 of the present disclosure, the secretome affects brain tumour growth and survival of the mice. At five weeks from implantation of tumour cells into mice brains, 41.2% of the secretome group were dead/sick, while only 18.8% of the standard media group were dead/sick.

In some examples, non-brain tumour models may include endothelial/blood vessel iPSCs co-cultured with non-brain tumour cells in secretome versus standard media. In some examples, non-brain tumour brain cells may include but is not limited to Alzheimer cells, Parkinson cell types, neuronal cell types, neuro-degeneration cell line models.

In some examples, non-tumour brain models may include a group of mice with secretome injected and no tumour cells. In some examples, the non-tumour brain models can be used to study blood vessels and/or brain tissue (such as brain parenchyma) changes, and the effects of the secretome on brain cells neurodegeneration and/or neuroregeneration.

In some examples, the present disclosure includes the use of in vitro models of blood vessels such as induced pluripotent stem cells (iPSCs). In some examples, blood vessel formation (such as patterns and quantity) was compared when grown in secretome versus standard media.

In some examples, the present disclosure uses a 3-dimensional in vitro model for such as but is not limited to drug screening, morphology/microscopy, creating composites to study interactions with blood vessels/fibroblasts, and the like. In some examples, the 3-dimensional in vitro model includes a 3-dimensional gel with the secretome of the present disclosure.

In another aspect, there is provided a method of culturing a proliferative cell comprising culturing the proliferative cell in a composition of the present disclosure.

In some examples, the method of the present disclosure comprises observing the expression of markers in the proliferative cell in the presence of the composition of the present disclosure.

In some examples, the markers expressed in the proliferative cells may include but is not limited to MELK, kinesin-like protein (KIF11/Kinesin-5/Eg5/BimC), and the like. The inventor of the present disclosure showed that MELK is enriched among patient ATRT tumours across three ATRT subtypes.

In some examples, the method of the present disclosure comprises observing morphology changes of the proliferative cells.

In some examples, morphology of the proliferative cell may include but is not limited to, clusters in suspension, stringy chains, web-like structures (stretching across long distances in the culture flask), interlacing strands, and the like. In some examples, morphology of the proliferative cells changes from clusters in suspension to stringy chains in the presence of the secretome.

Without wishing to be bound by theory, the inventor of the present disclosure found that the secretome as described herein is capable of maintaining tumour cells in a web-like interlacing structures that therefore keep tumour cells together/alive in this structure. It is believed that the secretome may possibly be involved in cell polarity maintenance to inform tumour cells to line up in such a manner.

In another aspect, there is provided a method of identifying a secretome phenotype of a disease, comprising culturing a diseased cell with an immune cell (such as a CD11b expressing macrophage), obtaining/extracting the media in which the cell is cultured to thereby obtain the secretome, analysing and/or mapping the identity of the individual component of the secretome to thereby provide a phenotype of the secretome of the disease.

In some examples, there is provided a method of identifying the secretome phenotype of a disease, comprising extracting RNA and performing methods known in the art (such as but is not limited to RNA-seq, reverse transcriptase polymerase chain reaction (RT-PCR)) for measuring transcript expression or protein expression (such as but is not limited ELISA).

In some examples, the disease as described herein may comprise a proliferative disease.

The proliferative disease as described herein includes tumour and/or inflammatory disease.

In some examples, the tumour may be a benign tumour. In some examples, the tumour may be a malignant tumour or cancer. In some examples, the disease is a cancer including, but not limited to, breast cancer, lung cancer, colorectal cancer, and the like. In some examples, the disease is cancer such as, but not limited to, brain tumour, medulloblastoma, embryonal brain tumour (atypical teratoid rhabdoid tumour (ATRT)), germ cell tumour, and the like. In some examples, the cancer includes tumour subclasses and molecular/genomic subtypes that tend to relapse such as, but is not limited to, solid cancers, liquid cancers, and the like. In some examples, solid cancers that tend to relapse my include but is not limited to, atypical teratoid rhabdoid tumour (ATRT), and the like. In some examples, liquid cancers that tend to relapse may include but is not limited to, leukaemia, lymphoma, and the like.

In some examples, the proliferative disease may be inflammatory disease such as an acute inflammatory disease and/or a chronic inflammatory disease. In some examples, chronic inflammation/chronic inflammatory disease acute inflammation/acute inflammatory disease may include but is not limited to ulcerative colitis, Crohn's disease, infectious disease, and the like.

In some examples, the infectious disease may be caused by a bacterial pathogen, a viral pathogen, a fungal pathogen, or a parasite.

Examples of a bacterial pathogen may include, but is not limited to, Escherichia coli, Mycobacteria spp, Salmonella spp, Staphylococcus spp, Clostridium difficile, Listeria monocytogenes, Group B Streptococci, vancomycin-resistant Enterococci (VRE), and the like.

Examples of a viral pathogen may include, but is not limited to, Human papillomavirus, Rhinovirus, Human cytomegalovirus in HIV-1 positive patient, Hepatitis virus, Coronavirus (CoV), severe acute respiratory syndrome (SARS), monkey pox virus and the like.

Examples of a fungal pathogen may include, but is not limited to, Botrytis cinerea, Pseudomonas syringae, Fusarium oxysporum and the like.

Examples of a parasite may include, but is not limited to, Leishmania parasites, Giardia, Cryptosporidum, Entamoeba and the like.

In some examples, the inflammatory disease may be a neurodegenerative disease, such as neuroinflammation that leads to neurodegeneration/brain damage/nerve damage. In some examples, the disease may be a neurodegenerative disease such as dementia, Alzheimer's disease, Parkinson's, Multiple Sclerosis, and the like. In some examples, the inflammatory disease may include but is not limited to fibrosis, muscle aging, and the like.

In some examples, the disease is brain tumour.

In some examples, the disease is atypical teratoid rhabdoid tumour (ATRT).

In some examples, the disease is medulloblastoma.

In some examples, the ATRT includes molecular subtypes of such as but is not limited to, MYC-ATRT, tyrosinase-ATRT (TYRATRT), sonic hedgehog-ATRT (SHH-ATRT), and the like.

The inventor of the present disclosure uncovered specific molecular subtypes of ATRT that were secretome enriched, matching macrophage-enrichment patterns (MYCATRTs>TYR-ATRTs>SHH-ATRTs). In some examples, the disease may include but is not limited to cancer cell lines and/or non-cancer cell lines.

In some examples, cancer cell lines may include but is not limited to ATRT cell lines, medulloblastoma cell lines, and the like.

In some examples, the ATRT cell lines may include but is not limited to CHLA-04, CHLA-05, CHLA-02, BT-12, CHLA-06, BT-37, CHLA-266, and the like.

In some examples, the medulloblastoma cell lines may include, but is not limited to, Daoy, D341, CHLA-01, CHLA-01R, and the like.

In some examples, non-cancer cell lines may include but is not limited to skin cells, nerve cells, brain cells, and the like.

Without wishing to be bound by theory, each disease (such as each cancer type) type can be further subclassified into different molecular subtypes. Some molecular subtypes are secretome phenotypes and others are not. As shown in the experimental data in FIG. 2A to 2D, using ATRT as an example, certain molecular subtypes have their unique secretome phenotype. Therefore, it is envisaged that individual patient tumours can be checked for expression of the secretome markers to identify secretome enriched tumours. This allows development of personalized therapies for precision medicine by matching of drugs with individual secretome phenotypes. Drug testing could also be performed to improve drug effects on individual secretome phenotypes.

The laboratory of the inventor of the present disclosure has the screening capability to identify secretome phenotypes for each cancer type to develop personalized therapies for precision medicine. Personalized screening for individual patient tumour can be performed for drug testing. Individual patient tumours can be directly checked if they are secretome phenotypes to match with drugs that work well in secretome phenotype tumours and to improve drug effects.

In some examples, the culturing a diseased cell with an immune cell is for such as but is not limited to, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, 48 hours, 3 days, 4 days, 5 days, 6 days, 1 week, 2 weeks, 3 weeks, 4 weeks, and the like.

In another aspect, there is provided a pan-cancer secretome atlas comprising a database of a plurality of secretome phenotype of a plurality of disease.

In some examples, the disease comprises a plurality of cancers, optionally 2, 3, 4, 5, 6, 7, 8, 9, 10, 11, 12, 13, 14, 15, 16, 17, 18, 19, 20, 21, 22, 23, 24, 25, 26, 27, 28, 29, 30 31, 32, 33, 34, 35, 36, 37, 38, 39, 40, or more cancers.

In some examples, the disease comprises 37 cancers. In some examples, the 37 cancers in the database of the pan-cancer secretome atlas include adrenocortical carcinoma, bladder urothelial carcinoma, breast invasive carcinoma, cervical squamous cell carcinoma and endocervical adenocarcinoma, cholangiocarcinoma, colon adenocarcinoma, colorectal adenocarcinoma, lymphoid neoplasm diffuse large B-cell lymphoma, glioblastoma multiforme, glioma, head and neck squamous cell carcinoma, kidney chromophobe, pan-kidney cohort (KICH+KIRC+KIRP), kidney renal clear cell carcinoma, kidney renal papillary cell carcinoma, acute myeloid leukaemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, testicular germ cell tumours, thyroid carcinoma, thymoma, uterine corpus endometrial carcinoma, uterine carcinosarcoma, uveal melanoma, intracranial germ cell tumours, atypical teratoid rhabdoid tumour and medulloblastoma.

In some examples, the pan cancer secretome atlas may include diseases with secretome phenotype such as but is not limited to cancer, acute/chronic inflammation disease, and the like.

As shown in the experimental data, the inventor of the present disclosed developed a Pan-Cancer Secretome Atlas which can be used to survey 37 types of adult cancers in genomic databases. This Pan-Cancer Secretome Atlas complements the secretome product and allows for identification and selection of cancer types which are secretome phenotypes. The inventor of the present disclosure found that the secretome of the present disclosure is elevated in patient tumours across many cancer types in the human body in comparison to matched normal tissue controls. Pharmaceutical companies/researchers can use the in-house Pan-Cancer Secretome Atlas to select specific cancer types and/or non-cancer inflammatory diseases to test their new drugs/chemical agents, using the secretome product screen of the present disclosure. Pharmaceutical companies can also screen large in-house compound libraries on cancer cell lines and/non cancer cell lines selected through the atlas of the present disclosure.

In some examples, gene expression of secretome-grown medulloblastoma and ATRT cells can be compared with standard media grown medulloblastoma and ATRT cells with methods known in the art (such as but is not limited to RNA-seq, whole genome sequencing). In some examples, telomere profiling may be done (such as with Pacbio) using the secretome of the present disclosure. In some examples, changes in secretome-grown versus standard media grown tumour cells may be compared (such as but is not limited to using Full moons biosystems/Phosphoexplorer array) to check for additional proteins in secretome. In some examples, cytokine levels in the secretome may be assessed using a cytokine profiling antibody array (such as but is not limited to Full moons biosystems/Phosphoexplorer array). In some examples, cryopreservation using secretome-based freezing media for extended durations compared with standard freezing media may be done, to check viability upon retrieval, and cell proliferation over time upon retrieval.

Also disclosed are composition or method as described herein.

In summary, the inventor of the present disclosure created an in-vitro assay to derive the secretome from macrophages. The inventor of the present disclosure matched this secretome against different paediatric brain tumours and molecular subtypes of ATRT using large patient cohorts of expression profiling data, compared against normal brain controls. A broad panel of six ATRT tumour cell lines were cultured in macrophage-derived secretome to establish its mechanistic effects of growth proliferation on ATRT cells. The inventor of the present disclosure found maternal embryonic leucine kinase (MELK) highly expressed across ATRT-subtypes, and expression was low in normal brain. OTSSP167, a Phase I/II molecular inhibitor of MELK, was tested on ATRT cell lines. The inventor of the present disclosure investigated the effects of secretome on OTSSP167 drug efficacy on ATRT cells. The inventor of the present disclosure has also found that MELK is highly expressed in medulloblastoma and tested OTSSP167 on five medulloblastoma cell lines. As shown in FIG. 41, the effects of both OTSSP167 and ispinesib were tested in secretome on medulloblastoma. FIG. 41A to 41C shows the secretome protecting tumor cells from drug killing in medulloblastoma cell lines that propagate as neurospheres (stem-like).

The present disclosure includes the identity of the secretome: (CCL2/MCP-1, MIP-1α/MIP-1β, CCL5/RANTES, MIF, IL-1ra/IL-IF3, IL-8) and the method to generate the secretome. Human myeloid-derived cells U937 were chemically stimulated using a mixture (PMA+IL-4) to generate (pan-macrophage) CD11b+ cells in high quantities. The six-factor secretome was harvested from these chemically induced cells. These 6 factors (CCL2/MCP-121,22, MIP-1α/MIP-1β22,23, CCL5/RANTES22,24-26, MIF27-29, IL-1ra/IL-IF330, IL-826) have been independently described by studies known in the art to affect macrophages or tumourigenesis.

The inventor of the present disclosure discovered that the secretome was capable of supporting tumour cell proliferation in ATRT and/or medulloblastoma during early tumour development and nutrient stress. This secretome was equal to standard media in preventing cancer cell death. These findings indicate that this six-factor secretome can sustain a critical mass of cancer cells despite lack of nutrients. Further, the inventor of the present disclosure uncovered specific molecular subtypes of ATRT that were secretome-enriched, matching macrophage-enrichment patterns (MYC-ATRTs>TYR-ATRTs>SHH-ATRTs). High-relapse-risk subtypes were concurrently secretome-enriched and macrophage-enriched. The inventor of the present disclosure found MELK enriched among patient ATRT tumours and a universal target across three ATRT-subtypes. OTSSP167, a molecular inhibitor of MELK, was efficacious across a panel of six ATRT cell lines and on an aggressive orthotopic xenograft mouse model (p=0.032) created from an autopsied specimen from a patient with tumour dissemination. Presence of the secretome reduced drug efficacy during early tumourigenesis, providing a pro-survival niche for tumour cells during initial drug response.

The secretome-product of the present disclosure, when added to cancer cells in-vitro, enables cancer cells to survive in a harsh, nutrient deprived environment, and allows the cancer cells to proliferate without nutrients. The secretome of the present disclosure was equal to standard media in preventing cancer cell death.

The inventor of the present disclosure found this secretome enriched in high risk tumours in several paediatric brain tumour types, include tumour subclasses that tend to relapse, as well as many adult cancers. The presence of the secretome-product of the present disclosure with cancer cells in-vitro: (a) decreases the efficacy of effective cancer drugs in an aggressive embryonal brain tumour occurring among infants and young children-atypical teratoid rhabdoid tumour (ATRT). This is relevant to patients and clinical trials testing novel drugs. (b) provides immune-rich conditions for laboratory testing of cancer cells. (c) is useful for testing/identifying new drugs on cancer cells/screening of compounds which are effective against cancer cells in an immune-rich in-vitro cultures, which mimics the microenvironment conditions in patient tumours. (d) enables us to identify cancer cells which may be more responsive to immune microenvironment modulation. (e) allows us to evaluate if new drugs may be potentially effective in immune-rich patient tumours. This is a critical step-efficacy of drugs in immune-rich conditions are less well-understood-which may explain why some drugs found effective in the laboratory, fail when they are administered to patients during clinical trials. (f) provides a tool to enable testing of new drugs in-vitro in immune-rich conditions in the laboratory, prior to application in patients during a clinical trial. This will be useful in pharmaceutical industries.

In addition, the secretome-product of the present disclosure: (1) allows the inventor of the present disclosure to screen patient tumours which are more likely to respond to certain drugs (2) select patients with immune-rich tumours for clinical trials.

The results of the present disclosure indicate that the secretome provides a favourable tumour ecosystem that allows malignant cells to survive nutrient deprivation and other selection pressures such as drug response and is important in high-relapse-risk subtypes. This approach can be applied on other cancers. The inventor of the present disclosure found this secretome similarly enriched among other brain tumour types, as well as many adult cancers in genomics databases.

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” or “attached” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other, or element A may contain element B or vice versa.

The term “adjacent” used herein when referring to two elements refers to one element being in close proximity to another element and may be but is not limited to the elements contacting each other or may further include the elements being separated by one or more further elements disposed therebetween. For example, the cleavage compound as described herein cleaves the oligonucleotide (e.g., primer, probe, and the like) within or adjacent to the cleavage domain. Thus, the term “adjacent” means that the cleavage compound cleaves the oligonucleotide at either the 5′-end or the 3′ end of the cleavage domain. In some examples of the present disclosure, the cleavage reactions yield a 5′-phosphate group and a 3′-OH group.

The term “and/or”, e.g., “X and/or Y” is understood to mean either “X and Y” or “X or Y” and should be taken to provide explicit support for both meanings or for either meaning. Further, in the description herein, the word “substantially” whenever used is understood to include, but not restricted to, “entirely” or “completely” and the like. As used herein, the term “substantially no” or “very low” refers to a sequence homology of less than at least 20%, or 19%, or 18%, or 17%, or 16%, or 15%, or 14%, or 13%, or 12%, or 11%, or 10%, or 9%, or 8%, or 7%, or 6%, or 5%, or 4%, or 3%, or 2%, or 1%, or 0.9%, or 0.8%, or 0.7%, or 0.6%, or 0.5%, or 0.4%, or 0.3%, or 0.2%, or 0.1%, or 0.01% sequence homology to the target nucleic acid (for example any human gene). In some examples, the term “substantially no” or “very low” sequence homology refers to the control gene having substantially different sequence to the target nucleic acid (for example any human gene). In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc. ,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### EXPERIMENTAL SECTION

### Methods

### Immunohistochemistry

Immunolocalization of CD163 was performed on formalin-fixed paraffin-embedded primary pediatric ATRT samples using a commercially available anti-CD163 polyclonal antibody. An automated microscopy system was used. The stained sections were reviewed by a pathologist blinded to data/hypothesis associated with the investigation, at the beginning of the study. Sections of human tonsils served as both positive and negative controls, as monocytes stain positively for CD163 while lymphocytes stain negatively for CD163.

### Primary Tumour Specimens

Fresh tumour specimens were snap frozen in liquid nitrogen and stored at −80° C. Between 1994 and 2014, a total of 10 primary paediatric ATRTs collected, and a panel of 8 patient-derived ATRT cell lines from international collaborating institutions, were extracted for RNA, used for RNA-Seq and quantitative reverse transcription polymerase chain reaction (qRT-PCR) in the present disclosure. The patient tumour samples were treatment-naïve and obtained at diagnosis, prior to initiation of treatment.

### RNA-Seq, Gene Expression Microarray and Classification of SHH-ATRTs, MYC-ATRTs and TYR-ATRTs

RNA was extracted from fresh frozen patient ATRT tumour samples and a panel of 8 patient derived ATRT cell lines using TRIzol. Expression profiles of 10 primary human ATRTs, 8 ATRT cell lines and 10 control brain tissue RNA were generated using RNA Seq for the Discovery Set. For the Validation Set, CEL files of 49 patient ATRT tumours profiled were used. Subtype identity of each patient tumour was determined by subtype-specific gene sets published. Primary human ATRTs were classified into molecular subtypes, Sonic-hedgehog (SHH)-ATRTs, MYC-ATRTs and TYR-ATRTs.

### Transcriptome Sequencing (RNA-Seq)

Genome wide transcriptome analysis was performed for 28 samples (10 primary human ATRTs, 8 ATRT cell lines and 10 control brain tissue RNA) using a high-throughput sequencing system. Total RNA was extracted and treated with DNase I and mRNA were isolated using Oligo(dT) and are fragmented using the fragmentation buffer and the cDNA is synthesized. The short fragments are purified and resolved with EB buffer and added with single nucleotide A (adenine). The adapter sequences were added to the short fragments and the suitable fragments are selected for the PCR amplification. For QA/QC of the sample libraries the inventor of the present disclosure has used an automated electrophoresis platform and a Real-Time PCR System. The library is sequenced using a high-throughput sequencing system. Paired end 2×100 base pair reads were generated on a high throughput-sequencing system and on average 8.96 Gb bases of reads was generated from each sample. The low-quality sequencing reads were removed before performing downstream analysis and the clean reads were mapped to hg 19 reference genome using HISAT and on average 85.42% reads are mapped to the reference genome. After mapping sequenced reads to reference genome, the transcripts were reconstructed. A total of 23,672 coding genes were identified.

### RNA-Seq Data Analysis

Raw reads were subjected to quality control and the filtered clean reads were aligned and mapped to the reference genome using sequence alignment tools. For gene expression analysis, the clean reads were mapped to reference genome using a sequence alignment tool. Gene expression levels were calculated with RNA sequencing quantification tool. Statistical analysis was performed using an analysis software to identify genes that were differentially expressed between patient tumours and normal brain controls. The differentially expressed genes were generated using a false discovery rate (FDR) method, p-value<0.05 with fold change<2 as cut off.

### Microarray Data Analysis

The inventor of the present disclosure downloaded the gene expression microarray data of 49 ATRT patient tumours from a database. Metadata containing tumour subtype identity for the 49 ATRT samples were downloaded. ATRT patient samples of three subtypes (MYC-ATRT, SHH-ATRT and TYRATRT) were identified previously. Of 49 patient tumours, 15 tumours were MYC-subtype, 16 tumours were SHH-subtype, and 18 tumours were TYR-subtype. The 49 raw CEL files were imported into an analysis software. RMA background correction and quantile normalization were performed. The gene expression values were log2 transformed and used for downstream gene expression analysis.

### Statistical Analyses

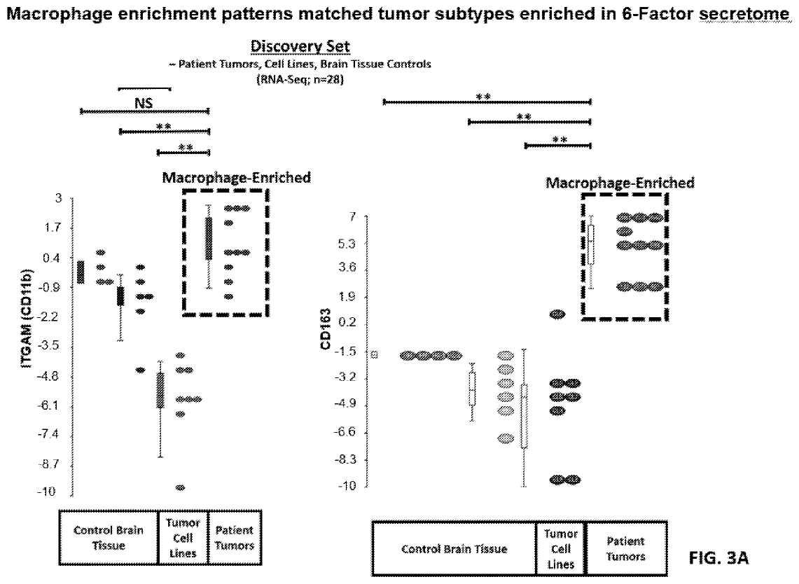

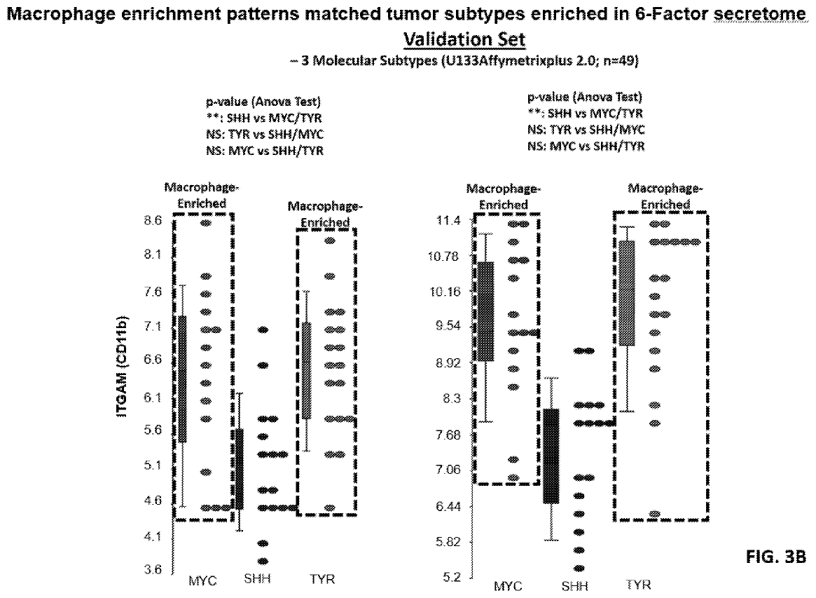

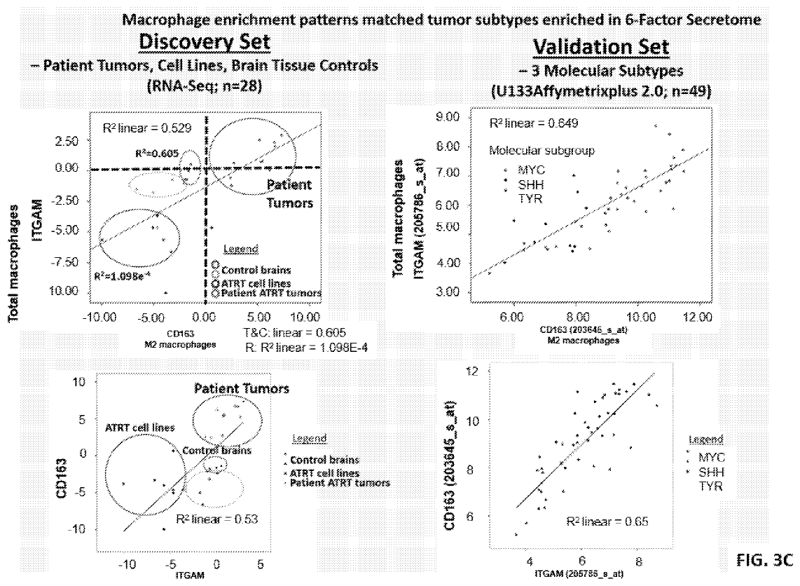

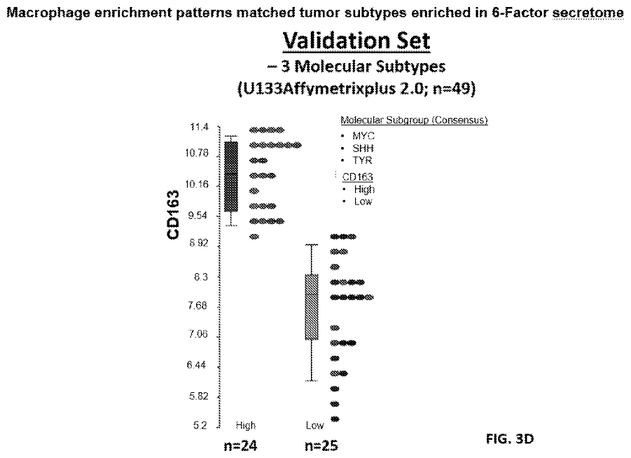

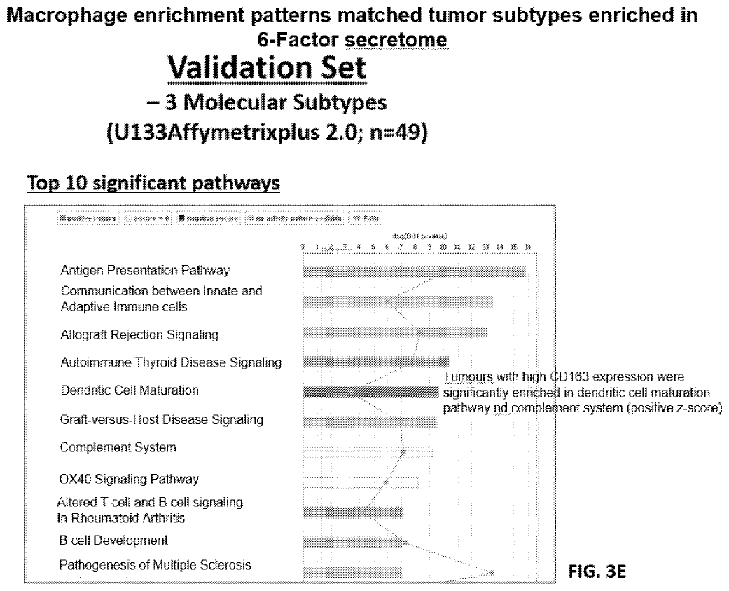

Statistical tests (such as student T-test) were used to compare marker expression between patient tumours, control brain tissues and cell lines. Statistical analysis was performed to identify gene to gene correlation of markers. The cut-off of p-value<0.05 was used to identify significant canonical pathways. −log(p-value) greater than 1.3 represents p<0.05. z-score represents the degree of observed changes in gene expression level (increase/decrease), indicating if the expected changes are associated with pathway activation or inhibition. An activation Z-score (Z-score<2)(FIG. 3E) indicates that the pathway is predicted as activated, while an inhibition Z-score (Z-score<−2) indicates that the pathway is predicted as inhibited.

### Quantitative Real-time Polymerase Chain Reaction

Total RNA was extracted from cultured cells from 7 ATRT cell lines, 4 other embryonal brain tumour cell lines, using TRIzol Reagent and reverse transcription was performed using high-capacity RNA-to-cDNA kit. Real-time PCR was performed using a kit. Each sample was loaded in triplicates. SYBR-green primers used for the reactions were MELK (H_MELK_1,) and housekeeping gene, GAPDH (H_GAPDH_1). PCR was performed with initial polymerase activation at 95 for 2 min, followed by 40 cycles of amplification (denaturation at 95 for 5 s, annealing at 65 for 10 s and extension at 72 for 20 s). The relative expression for each mRNA was calculated by formula of 2^{-ΔΔCt}.

### Proliferation Assays (Standard Growth Media)

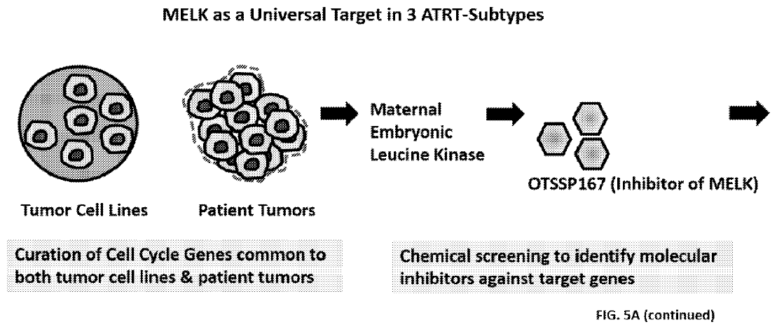

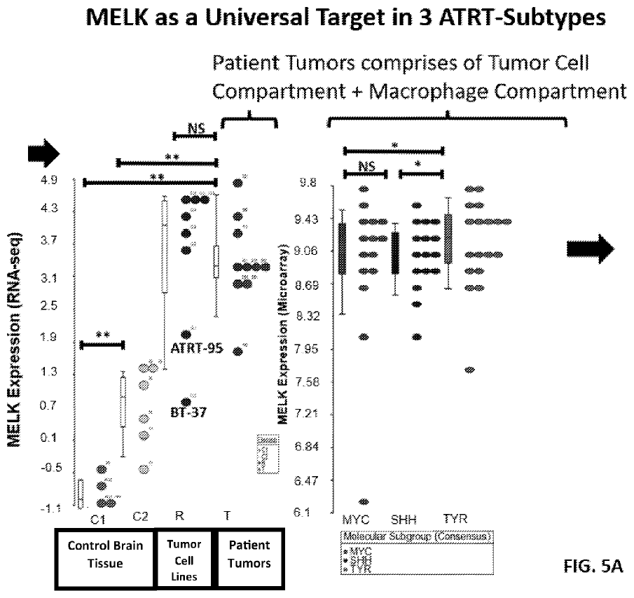

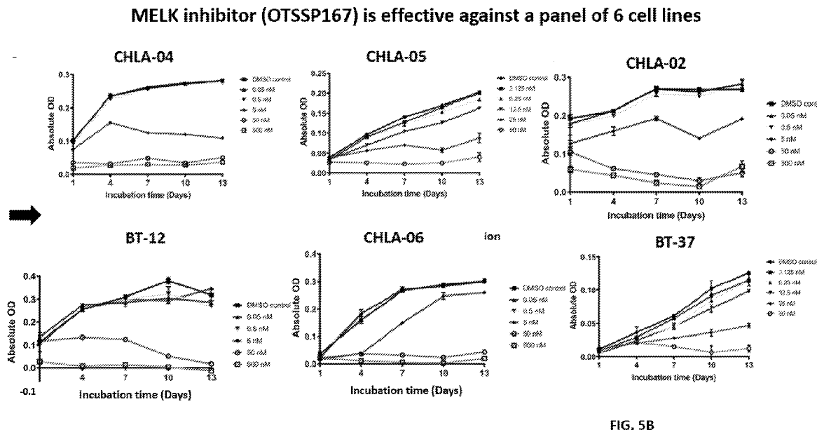

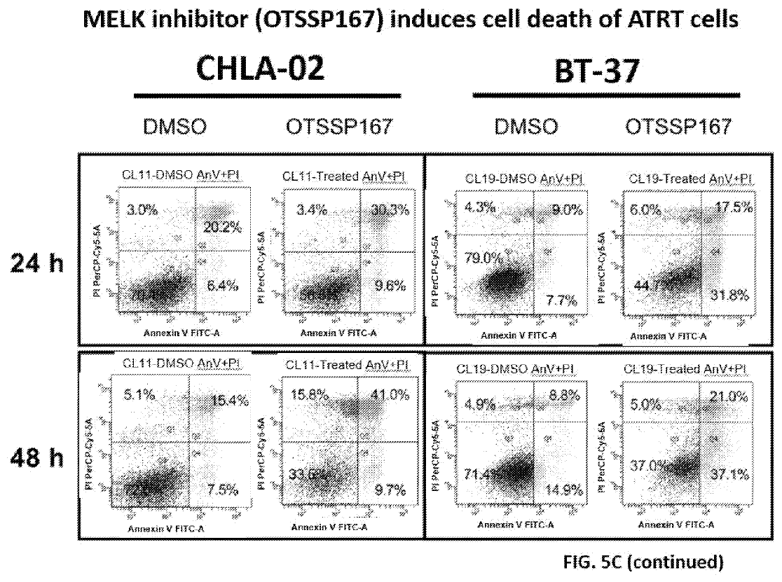

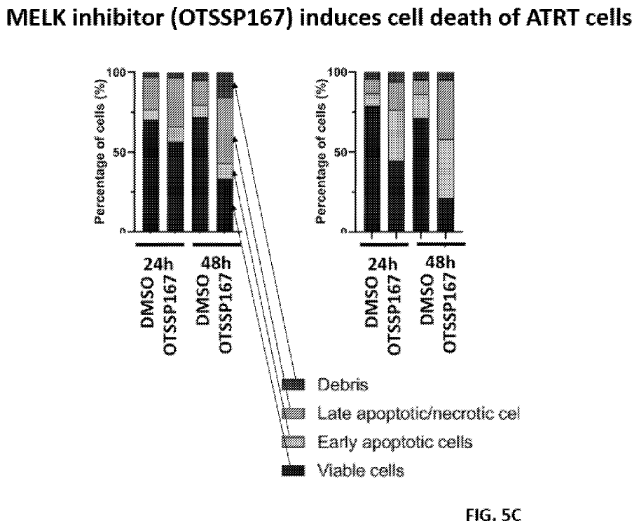

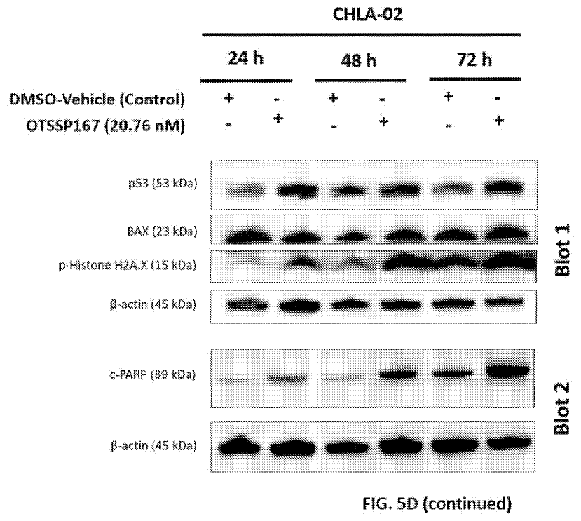

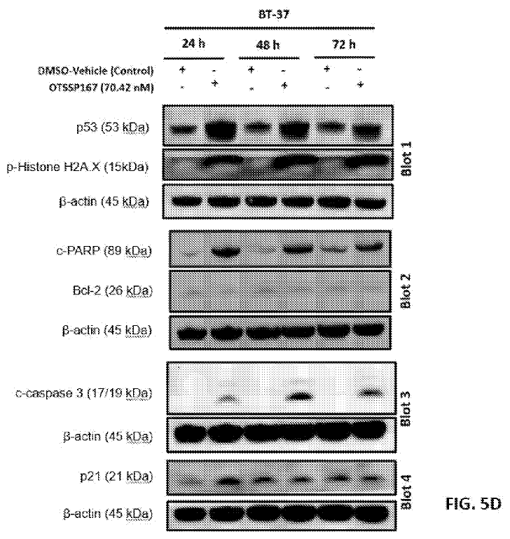

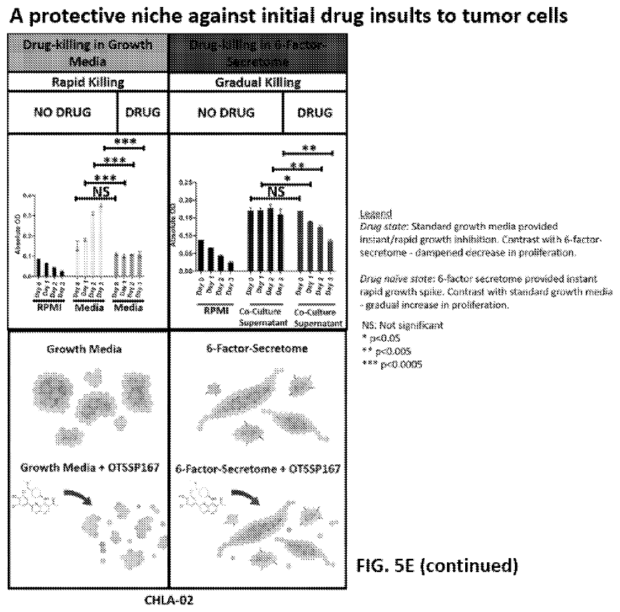

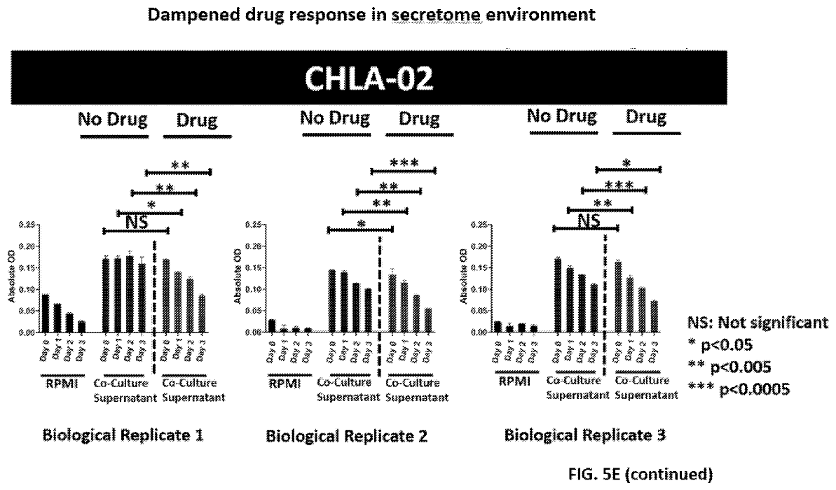

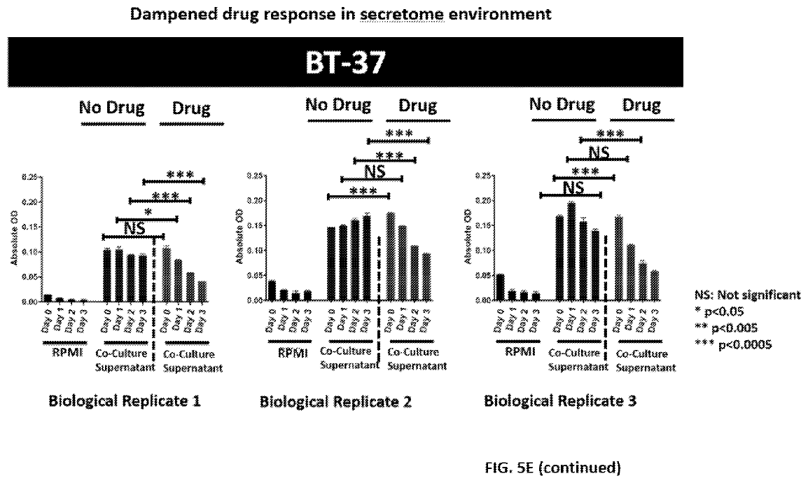

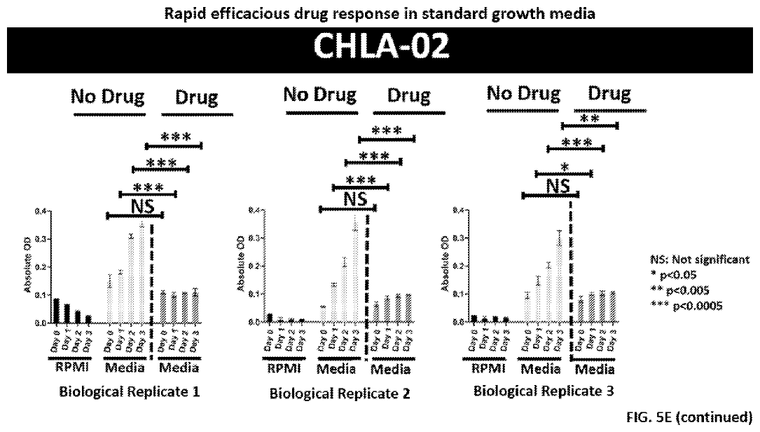

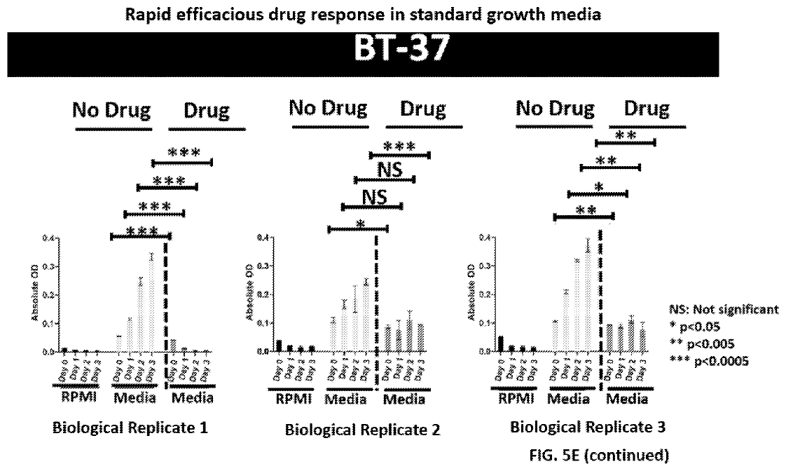

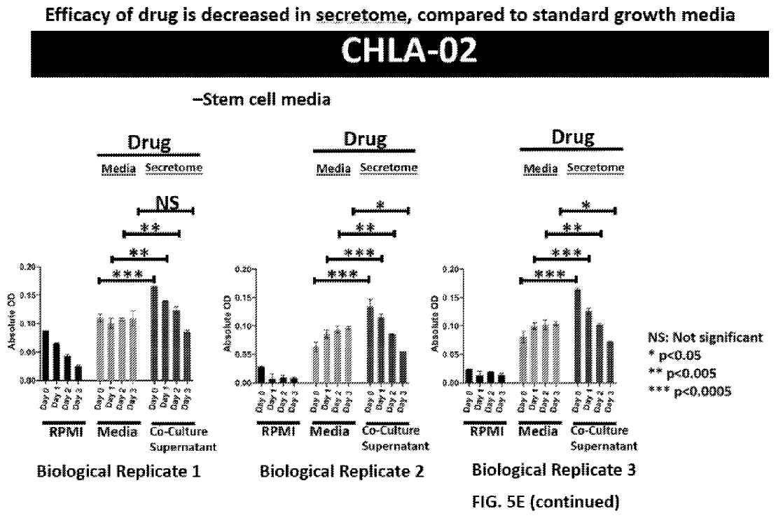

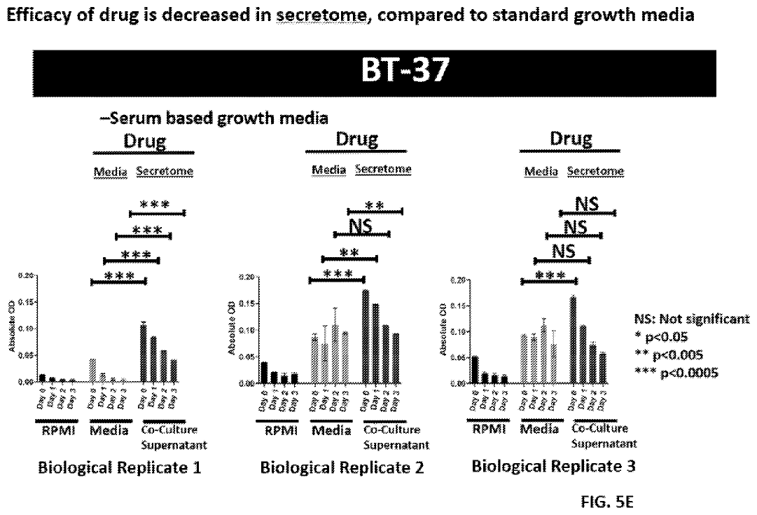

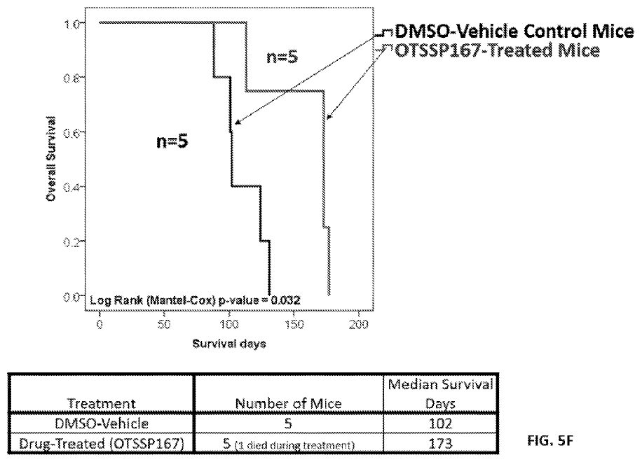

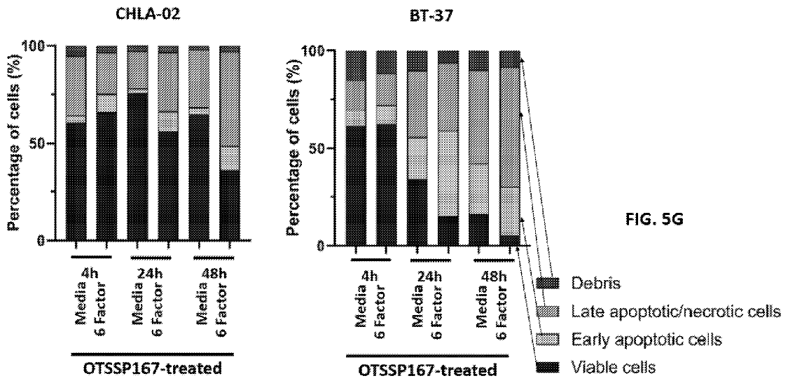

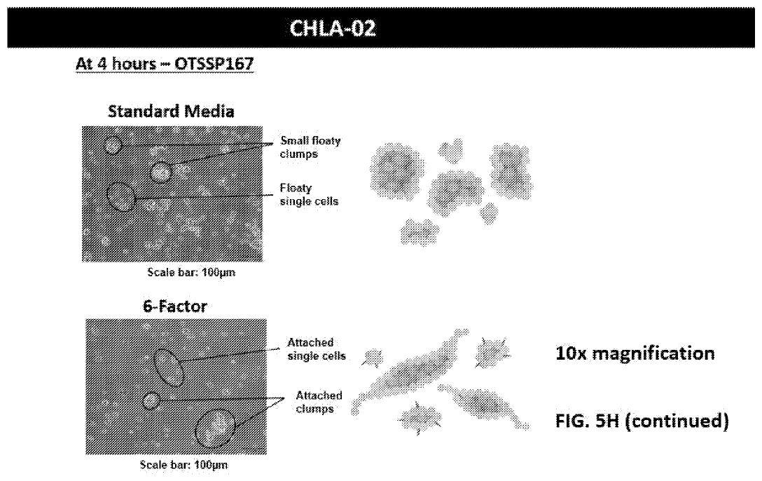

Tumour cells were seeded in quadruplicates for each treatment condition (FIG. 5B). OTSSP167 was added. Cell proliferation was determined, and optical density (OD) was measured using a fluorescence/luminescence detection System.

### Flow Cytometry

Tumour cells were treated in culture with OTSSP167 to detect apoptosis (FIG. 5C). At each timepoint, cells were harvested and stained with Annexin V-FITC and PI. Stained cells were then analysed by flow cytometry at the excitation wavelength of 488 nM (FITC) and 595 nM (PI). Cultured cells were treated with OTSSP167 to study the cell cycle changes. Cells were harvested and fixed with absolute ethanol. Fixed cells were stained with PI/RNase A master mix (PI and RNase A) prior to analysis by flow cytometry.

### Flow Cytometry Analysis of Macrophage Marker Expression

Human myeloid-derived cells (U937) were treated with PMA and different concentrations of IL-4 (or different combination of cytokines) and harvested after different time points of incubation. Cells were stained with PE-conjugated anti-human CD11b antibody or PE-conjugated anti-human CD163 antibody for 10 minutes. Isotype control, PE-conjugated REA control antibody was used to confirm antibody specificity. Stained cells were then analyzed by flow cytometry.

### Apoptosis Assay of OTSSP167-Treated Cells Cultured in Standard Growth Media Versus Six-Factor Secretome

Tumour cells were cultured in either standard growth media or six-factor-secretome for three different duration −4 h, 24 h, 48 h and 96 h (FIG. 5G). To study the killing effect of OTSSP167, tumour cells cultured in each condition (standard growth media or six-factor-secretome) were treated with OTSSP167. Cells were harvested at respective timepoints and stained with FITC-Annexin V and/or Propidium lodide. Stained cells were then analysed by flow cytometry at the excitation wavelength of 488 nM (FITC) and 595 nM (PI). A total of 10,000 cells were analysed per measurement.

### Serum-Free Supernatants

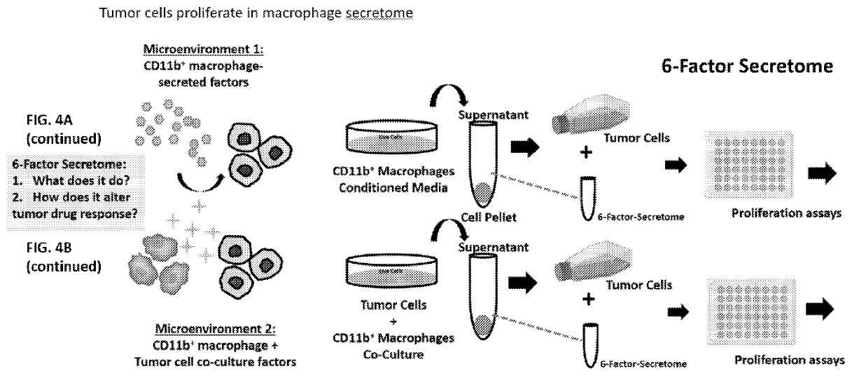

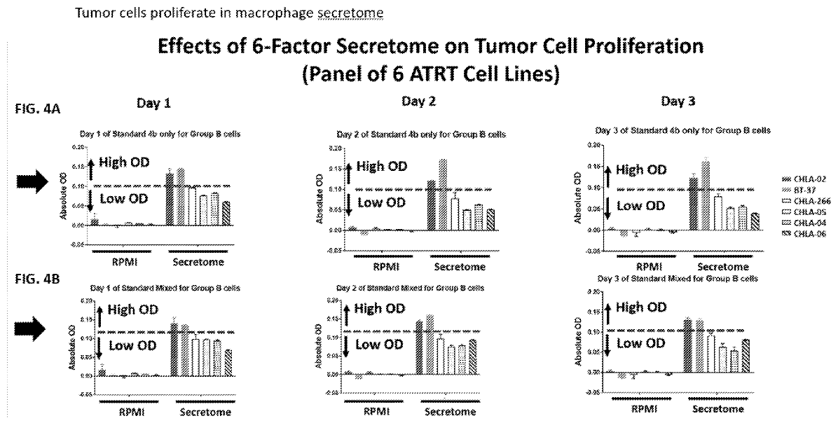

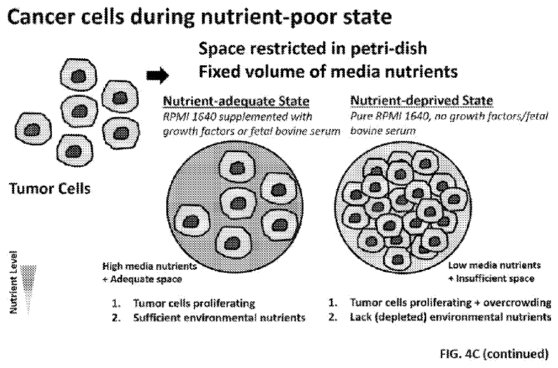

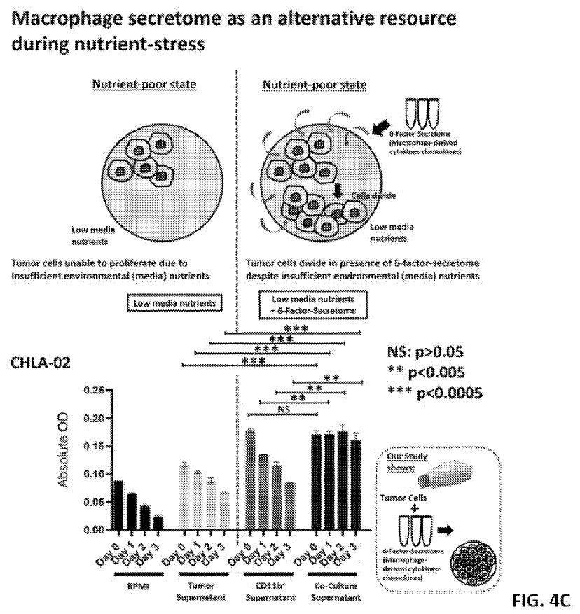

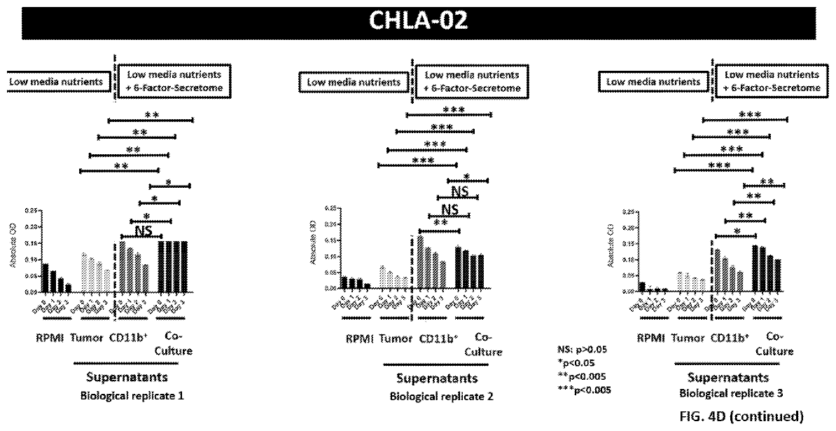

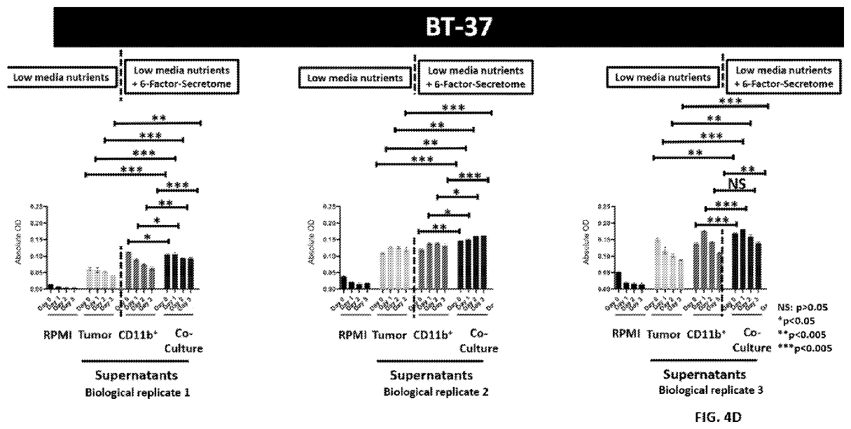

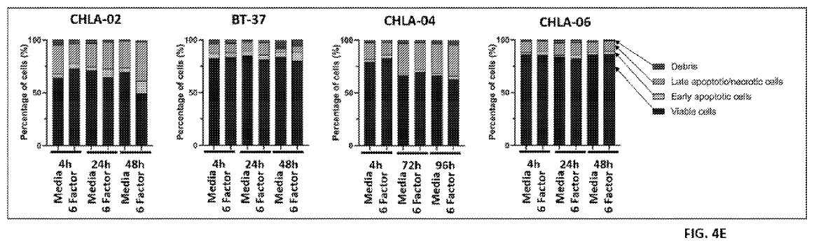

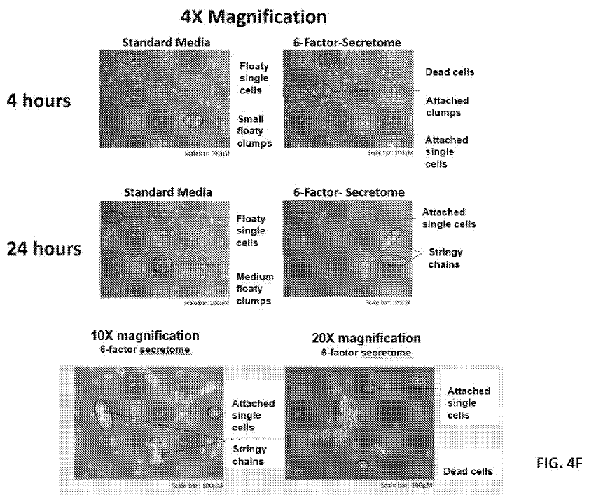

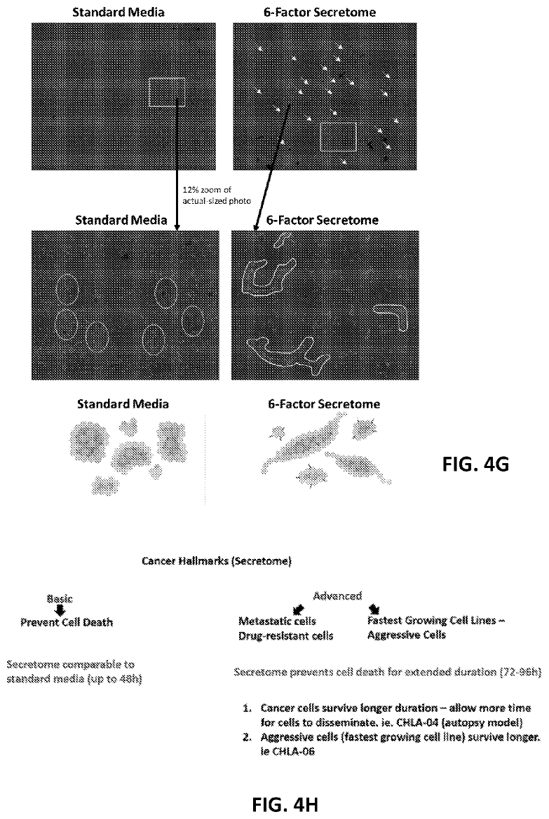

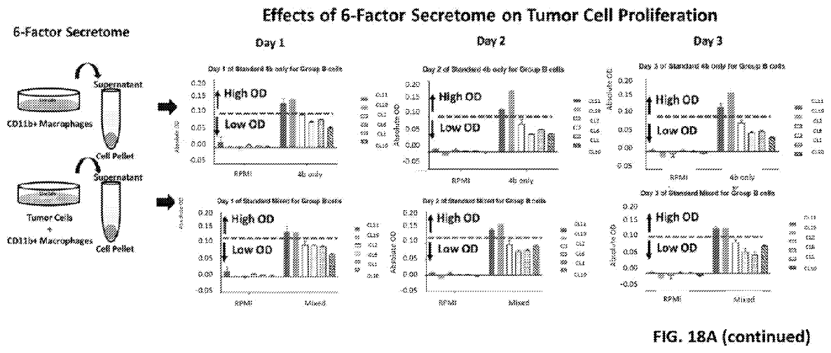

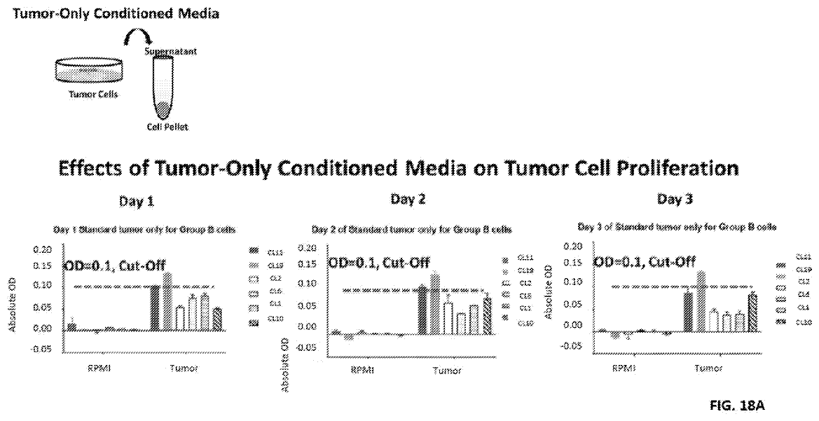

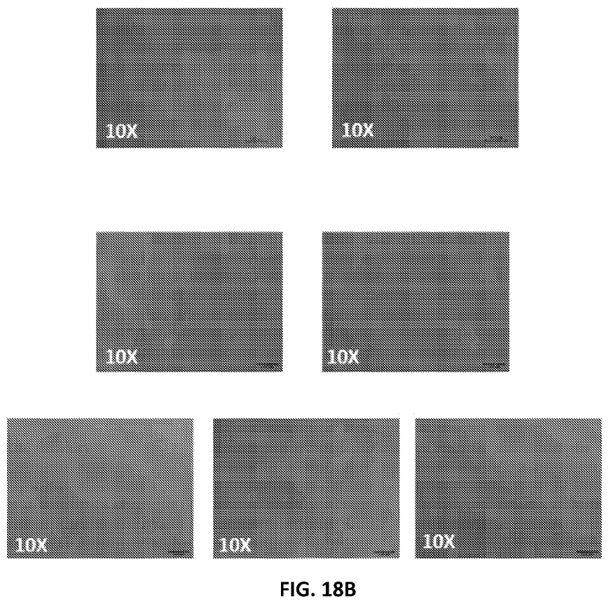

Human myeloid-derived cells (U937) were seeded at a fixed density and stimulated with PMA and IL-4 for 48 h to obtain CD 11b+ cells. Stimulated human myeloid-derived macrophages were harvested without trypsinization (Biological replicate 1 in FIG. 4D and FIG. 5E) and with trypsinization (Biological replicate 2 and 3 in FIG. 4D and FIG. 5E). These cells highly express CD11b+ with or without trypsinization (FIG. 14A). CD11b+ cells were washed twice with PBS. CD11b+ cells were plated at a fixed density in 1 ml of pure RPMI (serum-free) to obtain CD11b+ supernatants (Condition 2). CD11b+ cells (same density) were co-incubated with tumour cells at 1:1 ratio in pure RPMI (serum-free) to obtain co-culture supernatant (Condition 3). Tumour cells were seeded at the same density to obtain tumour supernatant (Condition 1). After 3 h of incubation at 37° C. and 5 % CO2, supernatants were harvested by centrifugation, verified cell-free (FIG. 18) and stored at −80° C. prior to further use.

### Cell Proliferation Assay (Secretome-Based or Growth-Media-Based)

Tumour cells were suspended in 3 types of cell-free supernatants: CD 11b+ supernatant, tumour supernatant, and CD11b+-tumour co-culture supernatant. Standard growth media and pure RPMI conditions were used as positive and negative controls respectively. Cells were seeded. For drug-treated conditions, cells were treated with either DMSO or OTSSP 167. Cell viability was determined by a kit at 4 h, 24 h, 48 h, 72 h time points, over a period of 3 days. Optical density (OD) was measured using a fluorescence/luminescence detection system (FIGS. 4A, 4B, FIG. 18). All other absorbance (OD) was measured at 450 nm using a multilabel plate reader. Absorbance of wells with serum-free pure RPMI was used as blank.

### Cytokine Profiling

Assessment of cytokines present in the cell supernatants were performed using a cytokine array. 700 μl of supernatants was used. The cytokine spots were visualized by an imaging system.

### Immunoblotting

Cells were lysed in RIPA buffer with 1% Protease and Phosphatase Inhibitor Cocktail on ice. Protein samples were first denatured with 5% β-mercaptoethanol in Laemmli's buffer at 95° C. Protein concentration was determined using Protein Assay Kit. Protein lysates were resolved, and equal amounts of denatured proteins was run on NuPAGE 4-12% Bis-Tris Protein Gel and transferred onto PVDF membrane. Membrane blots for phosphorylated Histone H2A.X were blocked with 5% Bovine serum albumin (BSA) in 1× TBS-T (10× Tris-buffered Saline, 0.1 % Tween® 20). Other membrane blots were blocked with 5% skim milk in 1× TBS-T for 1 hour and incubated with its respective primary antibodies at 4° C. overnight. Protein bands were visualized using an imaging system with Western blotting substrate. Primary antibodies used were: Eg5, cleaved PARP, β-actin, cleaved caspase-3, and p53, p-Histone H2A.X, p21. Secondary antibodies included: anti-rabbit IgG HRP-linked and anti-mouse IgG HRP linked.

### Statistical Analysis

All data were expressed as the mean ±SD (standard deviation). Statistical analysis was performed (such as unpaired student t test), to determine the statistical significance of differences observed between the groups. For results to be considered statistically significant, *p<0.05, **p<0.01, ***p<0.001.

### Orthotopic Implantation of Patient-Derived Tumour Cells Into Mouse Brains

Rag2/Severe combined immune deficiency (SCID) mice were bred and housed in a pathogen-free facility. Surgical implantation of tumour cells (1×10−5) into mouse cerebrum was performed using the method of the present disclosure previously described by studies in the art. The general recommendations in ARRIVE guidelines 2.0 (https://arriveguidelines.org/) were followed in the study of the present disclosure. The sample size of the present disclosure was small. The inventor of the present disclosure did not use any method to generate the randomisation sequence. The animals were randomly assigned into treatment versus control groups to have approximately equal number of animals per group. The inventor of the present disclosure has recently published that gender does not statistically affect treatment outcome, and both genders were included in the study of the present disclosure. The animals of the present disclosure were housed in the same location in side-by-side cages and managed in a standardized maintenance protocol by the animal unit and the team of the present disclosure to minimize any confounding effects, the inventor of the present disclosure did not employ any special strategy to minimize other confounders. Treatment was delivered to animals assigned in treatment group, cage-by-cage. Blinding was not possible as the inventor of the present disclosure have a small team of 2-3 individuals working with the animals and the inventor of the present disclosure are all involved at various stages and day-to-day processes of the animal work. Separate batches comprising of various litters were used, minimizing bias from the same litter effect. The inventor of the present disclosure has previously published using this animal protocol and is not otherwise deposited in any repository/registry.

### Intraperitoneal Drug Administration

OTSSP167 was dissolved in a solution of dimethyl sulfoxide, Kolliphor® EL and sterile water. A daily dose of 5 mg/kg of OTSSP167 was administered to the mice via intraperitoneal injection for 14 days. Ten animals were studied (5 treated, 5 controls). For Batch 94B, Ispinesib was dissolved in a solution of Cremophor EL, dimethyl sulfoxide, water. Ispinesib was administered intraperitoneally every four days for three doses, with the treatment course repeated on day 21. The dose of ispinesib was 10 mg/kg.

### Clinical Staging System to Monitor Mice Fitness

Mice were carefully monitored daily their fitness using a clinical staging system we previously published. When mice developed severe signs of neurologic deficit or became moribund, they were euthanized according to the protocol of the present disclosure.

### Statistical Analyses of Animal Survival

The survival of each mouse was taken to be the number of days from the date of tumour implantation to the date of death (overall survival), where event=1 and censored subject=0. Data were censored for mice that were alive at the time of analysis. All Kaplan-Meier survival curves were plotted, and the survival analysis was performed.

### Results

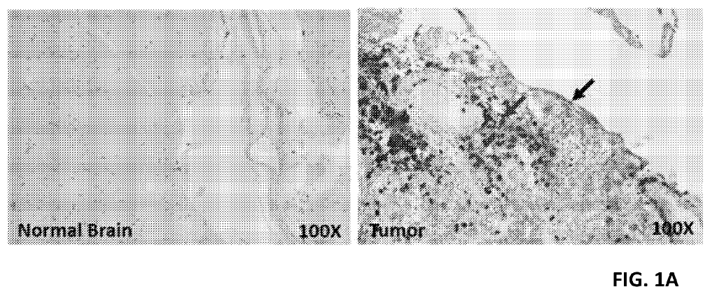

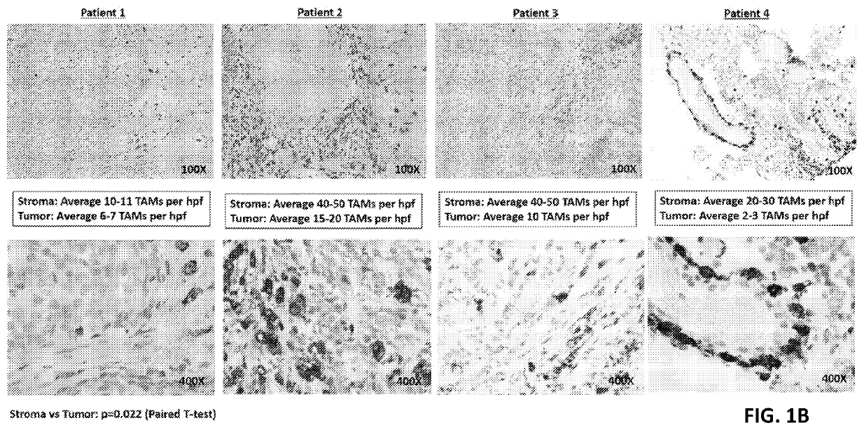

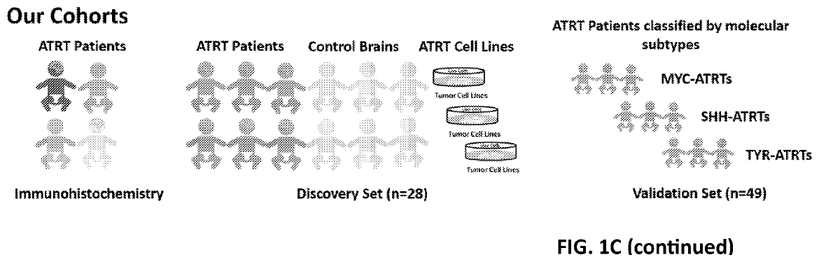

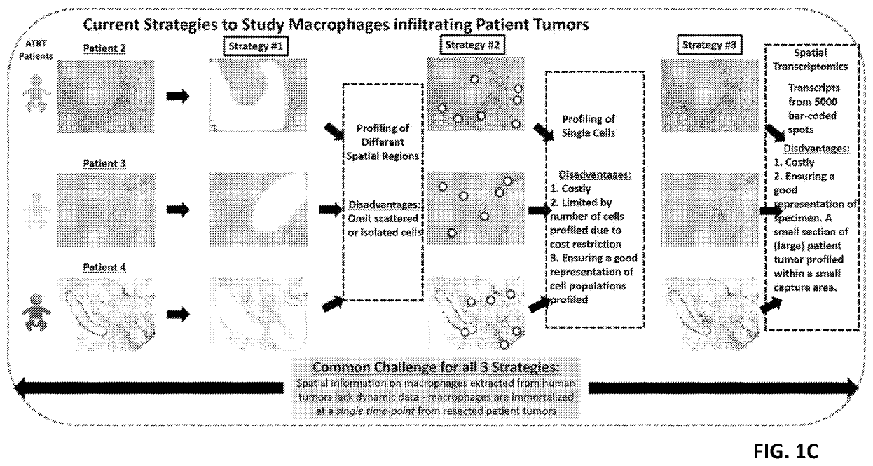

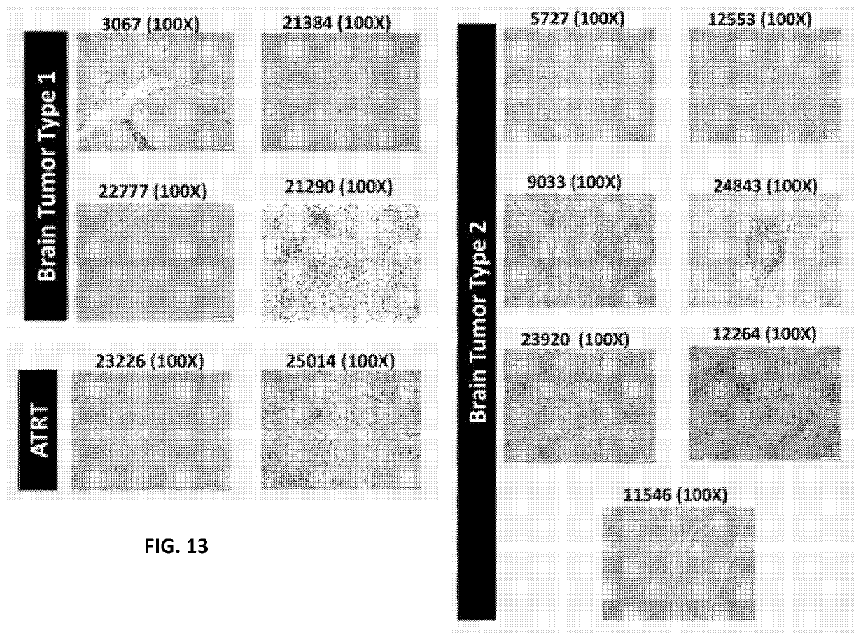

M2 macrophages have been implicated with poorer patient survival in medulloblastoma, another type of paediatric embryonal brain tumour. The inventor of the present disclosure first examined four patient ATRT tumours for macrophage infiltration with immunohistochemical staining using CD163 (M2 macrophage marker) monoclonal antibody. CD163 was exclusively expressed on alternatively activated or M2-polarized macrophages. Patient ATRT tumours depicted an architecture of macrophage compartment interfacing with tumour cell compartment; this macrophage compartment was clearly absent in normal brain tissue (FIG. 1A to 1B). FIG. 1A to 1B shows macrophage-tumour ecosystems depicting challenges to study infiltrative macrophages among patient tumours. Atypical teratoid rhabdoid tumours (ATRTs) exhibit an architecture of a macrophage compartment (absent in normal brain tissue) interfacing with the tumour cell compartment. Morphologically, CD163 was expressed on macrophages and not tumour cells. Macrophage-enriched compartment with high density of macrophages was nested at tumour border (edge of patient tumour resected from normal brain) (FIG. 1A). Normal brain (control) was barely infiltrated by macrophages (FIG. 1A). Intra-tumoural heterogeneity of macrophage infiltration within each single patient tumour (Patient 1-4) was consistently observed across patient tumours, demonstrating that stroma was distinctly infiltrated by high number of macrophages compared to tumour compartment (p=0.022, Paired T-test, FIG. 1B). Macrophages sometimes formed a well-defined perivascular ring suggesting origins related to circulating myeloid cells in Patient 4 (FIG. 1B). The inventor of the present disclosure surveyed a mix bag of 13 paediatric brain tumours from a single institution and found similar enrichment of CD163 macrophages among a good proportion of patient tumours (FIG. 13). The inventor of the present disclosure observed that not all patient tumours within each histological type demonstrated high CD163 expression—two ATRT tumours in this cohort, one demonstrated CD163hi expression and the other CD163lo (FIG. 13). This suggests that within a single histological type, individual patient tumours vary in macrophage infiltration.

Many strategies currently employed to study macrophages among patient tumours, explore the spatial relationship of macrophages in tumours immortalized at a single time-point (FIG. 1C), such as genomic profiling of micro-dissected regions of macrophages in patient tumours (Strategy 1), single cell sequencing of individual macrophage in patient tumours (Strategy 2) and spatial transcriptomics (Strategy 3). Challenges of Strategy 1 include omission of scattered, invasive macrophages (FIG. 1B) in patient tumour, whilst limitations of Strategy 2 include (1) high cost to sequence large numbers of single cells, (2) data limited by the number of cells sequenced (cost limitation), and (3) ensuring good representative population of single cells sequenced. The common problem of both Strategy 1 and 2, is that both approaches involve tearing apart the macrophage microenvironment compartment from the tumour compartment. The unifying challenge among all three strategies is that spatial information on macrophages extracted from human tumours lack dynamic data macrophages are immortalized at a single time-point from resected patient tumours, resulting in a loss of opportunity to study the in-situ, real-time interaction between the tumour and macrophage compartments. To approach this problem, a secretome was derived from human CD11b+ (pan-macrophage) cells (FIG. 2A). The inventor of the present disclosure first treated human-myeloid-derived cells with PMA-cytokine cocktail to induce CD11b (pan-macrophage marker) expression. CD11b (pan-macrophage marker) was consistently high 80-90% (FIG. 2A, FIG. 14A), whilst CD163 (M2 macrophage marker) expression was very low (FIG. 14B).

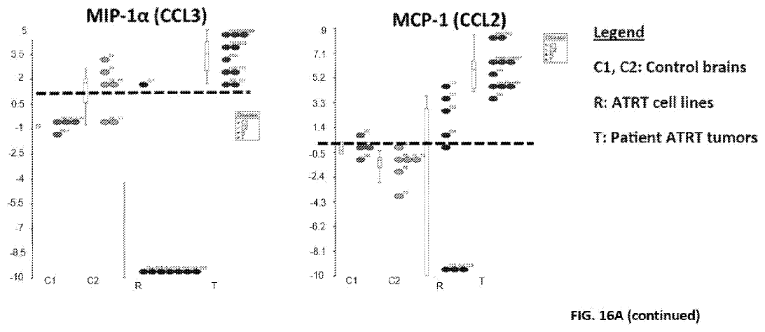

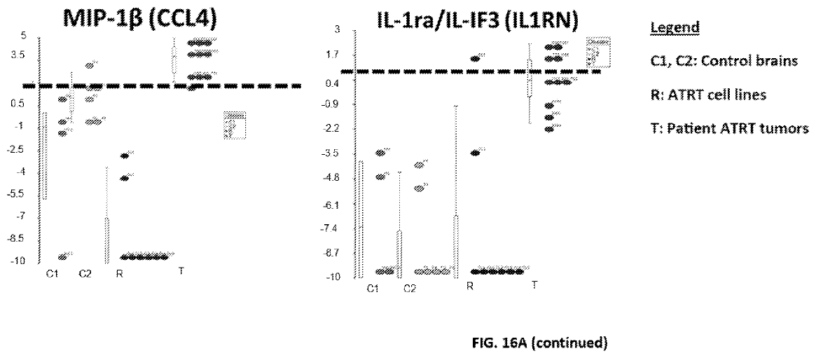

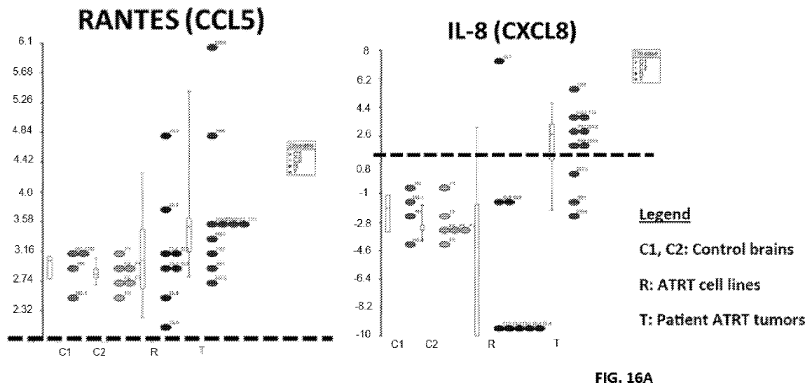

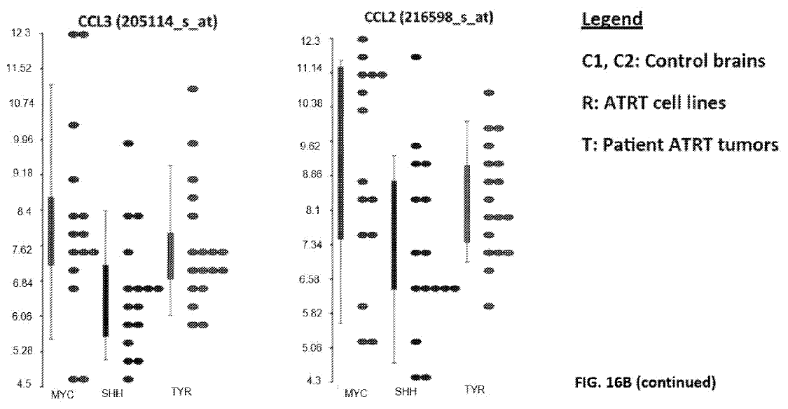

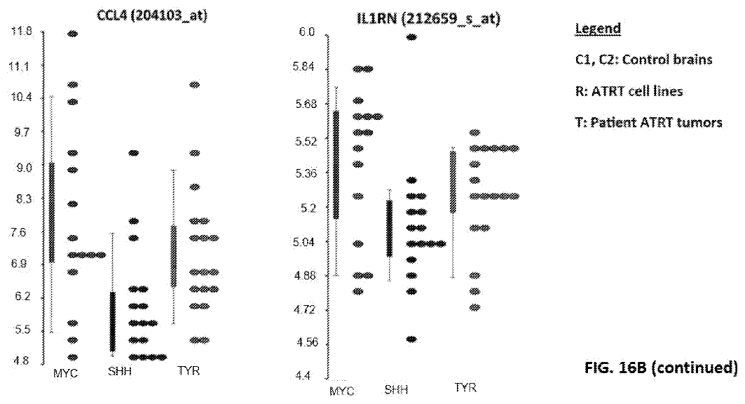

Array analyses of 36-cytokine-chemokines were performed to compare the secreted factors in 3 different conditions: (1) tumour cells only conditioned media, (2) CD11b+ cells only conditioned media, and (3) tumour-CD11b+ co-culture. Results demonstrated an enrichment pattern of 6 cytokines-chemokines released by CD11b+ macrophages in the microenvironment (FIG. 2B). The inventor of the present disclosure employed the most aggressive ATRT tumour cells (CHLA-06) from the panel of 7 ATRT cell line in the present disclosure to create the tumour compartment (FIG. 15), based on the assumption that the most rapid growing in-vitro cell line CHLA-06 will secrete the most complete spectrum of tumour-promoting factors. Importantly, presence of tumour cells enhanced the production of these 6 specific cytokines-chemokines (CCL2/MCP-1, MIP-1α/MIP-1β, CCL5/RANTES, MIF, IL-1ra/ILIF3, IL-8) by CD11b+ cells (FIG. 2B). Tumour cells in isolated culture did not produce this panel of cytokines (FIG. 2B) apart from MIF, indicating that this cytokine panel was unique to and released by the CD11b+ cell component. Comparing 10 patient tumours, 10 controls (normal brain tissue controls from various brain regions of cerebellum, cerebrum, pons) and 8 patient-derived cell lines in Discovery Set (RNA-seq, n=28 samples), this secretome was enriched only among patient tumours and not normal brain controls nor patient-derived tumour cell lines (FIG. 2C, FIG. 16A). Normal brain controls were distinctly secretome-bland (FIG. 2C). Remarkably, this enrichment pattern among patient tumours was recapitulated in the expanded 36-cytokine-chemokine panel (all candidates on cytokine array platform, FIG. 2B). Therefore, FIG. 2A and FIG. 2B show secretome derived from CD11b+ macrophages are enriched in specific molecular subtypes of ATRT.

The inventor of the present disclosure next compared the secretome enrichment patterns among patient tumours of three different ATRT molecular subtypes (MYC-ATRT, SHH-ATRT, TYR-ATRT) in Validation Set comprising of 49 patient ATRT tumours classified by molecular subtypes. MYC-ATRTs were the most enriched for secretome, followed by TYR-ATRTs, while SHH-ATRTs were almost devoid of secretome (FIG. 2D, FIG. 16B). Similarly, the enrichment pattern of the expanded 36-cytokine-chemokine panel (all candidates on cytokine array platform) recapitulated the secretome enrichment pattern among three ATRT molecular subtypes (MYC>TYR>SHH) and was evident only among patient ATRT tumours and not normal brain nor cell lines, in both Discovery (FIG. 2C) and Validation Sets (FIG. 2D).

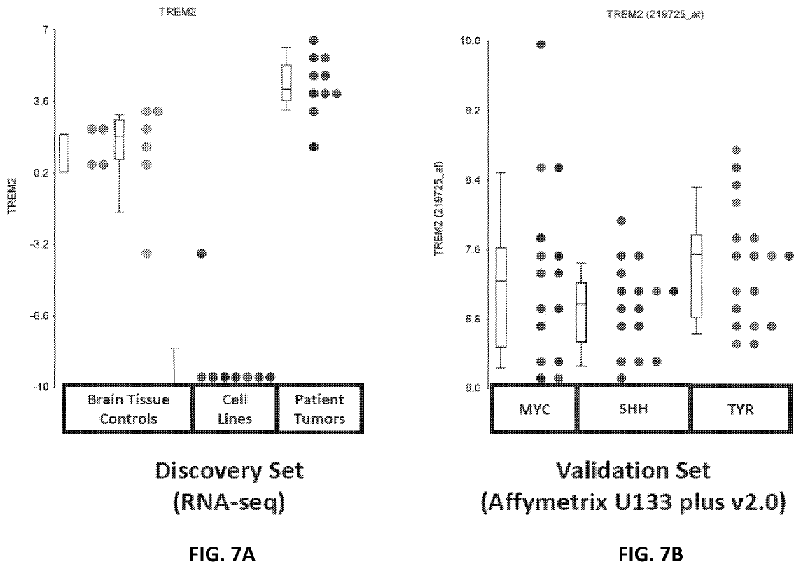

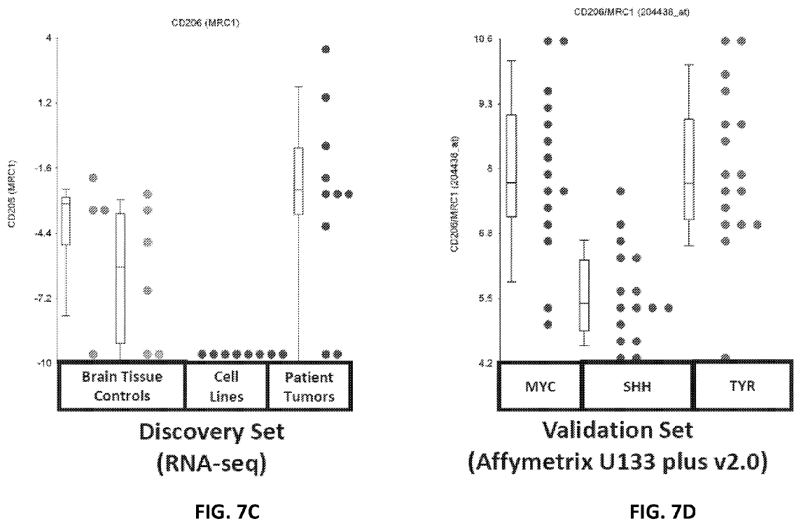

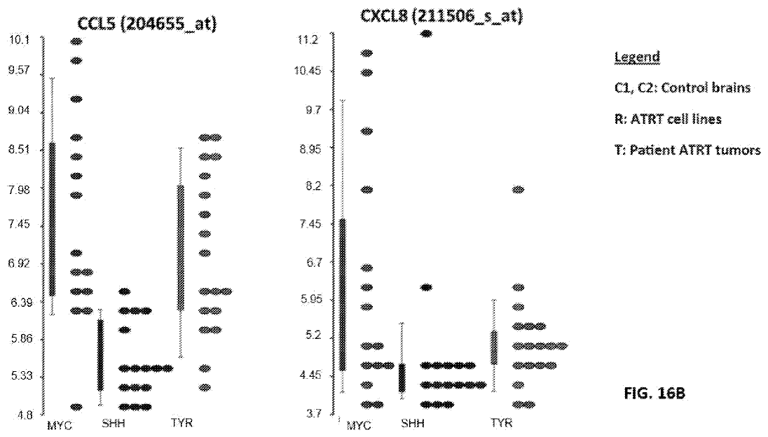

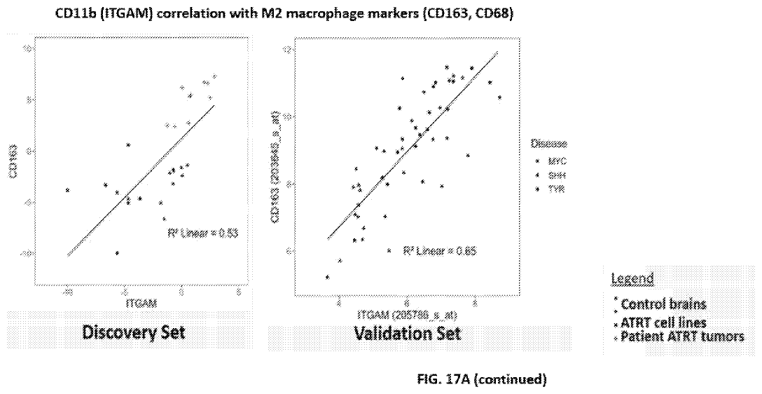

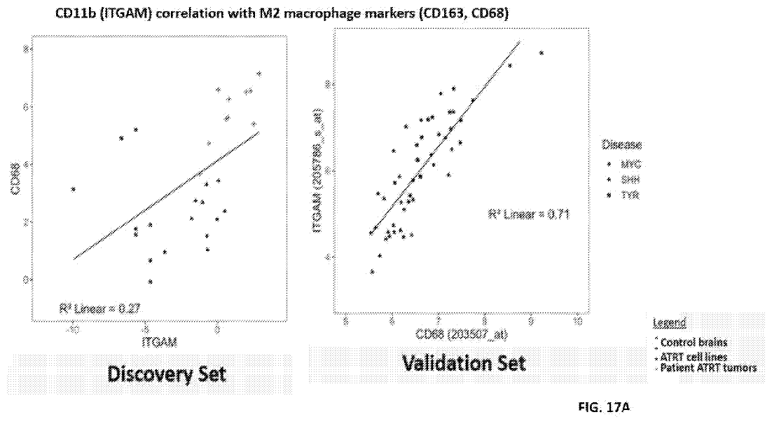

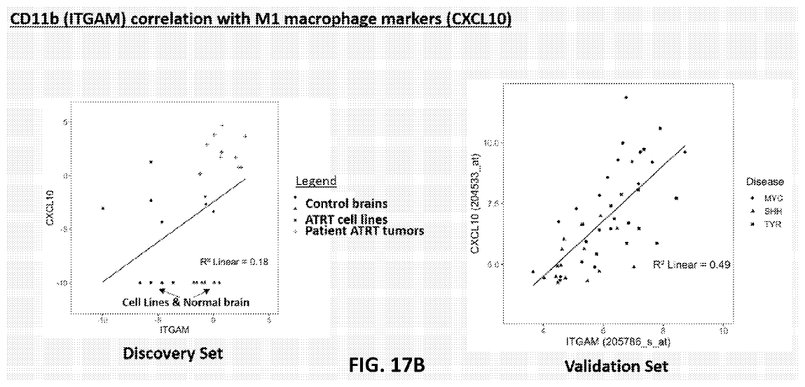

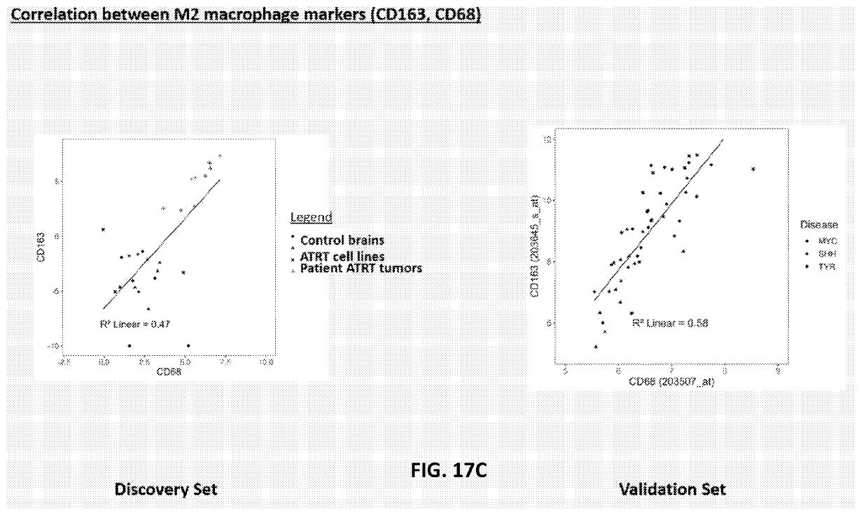

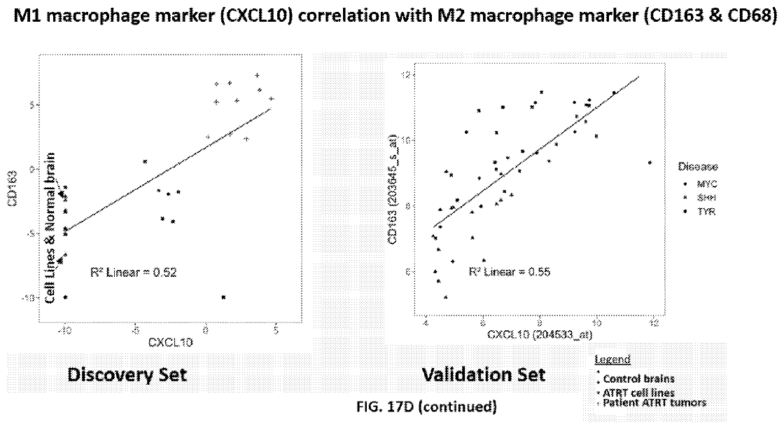

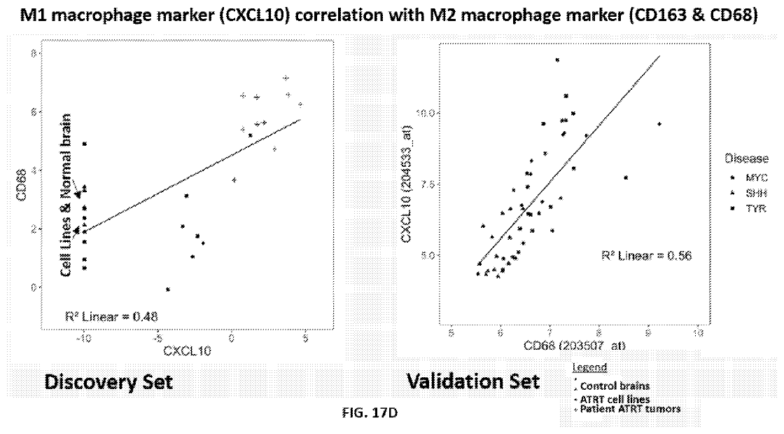

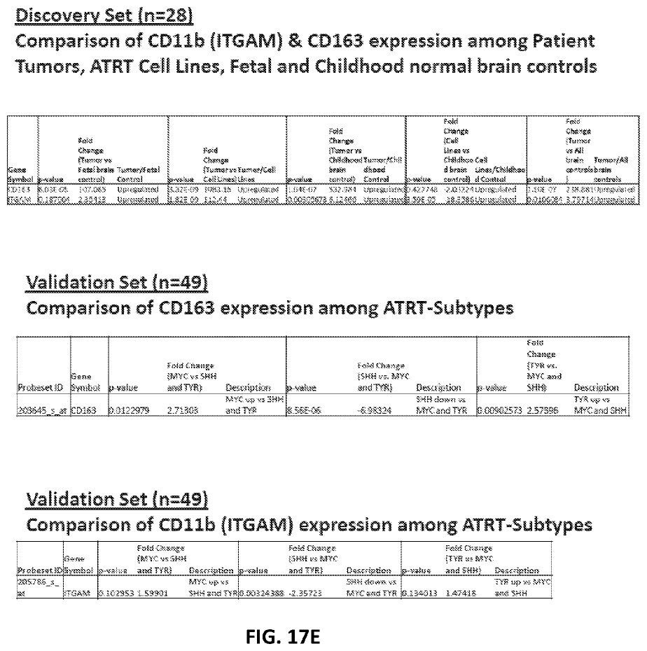

FIG. 3A to FIG. 3E show that macrophage-enrichment pattern in patient tumours matches secretome-enriched subtypes. Macrophage infiltration density further mirrored the enrichment patterns of secretome. Quantitative bioinformatic evaluation of macrophage markers among patient tumours, controls and patient-derived tumour cell lines in Discovery Set (RNA-seq, n=28 samples), confirmed that patient tumours were enriched in CD11b (pan-macrophage marker) and CD163 macrophages (M2 macrophage marker), but not tumour cell lines nor normal brain tissues (FIG. 3A). Control brains were comparably low in CD11b and CD163 macrophage-content. Macrophage markers of various subsets, specifically CD11b and CD163, correlated well among patient tumours but not in tumour cell lines nor normal brain tissues (FIG. 3B, FIG. 17). Among 3 molecular ATRT-subtypes in Validation Set, MYC-ATRTs and TYR-ATRTs were macrophage-enriched tumours (FIG. 3C-3D), matching secretome-rich subtypes, indicating distinct signalling pathways influencing macrophage recruitment and function in patient tumours of these secretome-rich subtypes. The inventor of the present disclosure further interrogated additional macrophage markers in both Discovery Set and Validation Set and observed similar trends (FIG. 7 to 11). FIG. 7A to 7D show expression of additional macrophage markers (TREM2 and CD206) in Discovery Set (n=28) and Validation Set (n=49). Dot plots indicating expression of TREM2 and CD206 in FIG. 7A and FIG. 7C, Discovery Set (n=28) by RNA-sequencing, and FIG. 7B and FIG. 7D, Validation Set (n=49) by Affymetrix U133 plus 2.0, classified by molecular subtypes. SHH-ATRTs were macrophage-low and secretome-low. Tumours displaying M2 macrophage-enriched phenotype (CD163hi) were frequently MYC-ATRTs and TYR-ATRTs and were significantly enriched in dendritic cell maturation pathway and complement system (FIG. 3E). These findings confirmed the immunohistochemical findings of the present disclosure that macrophages inhabit only patient tumours (FIG. 1A to 1B), but not normal brain tissue controls nor patient-derived tumour cell lines.

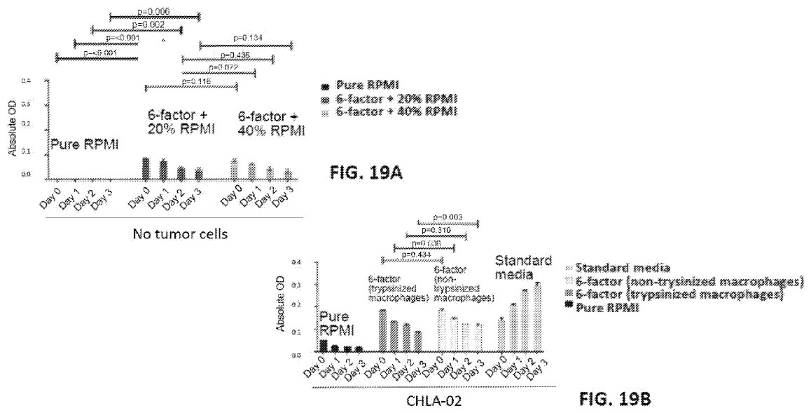

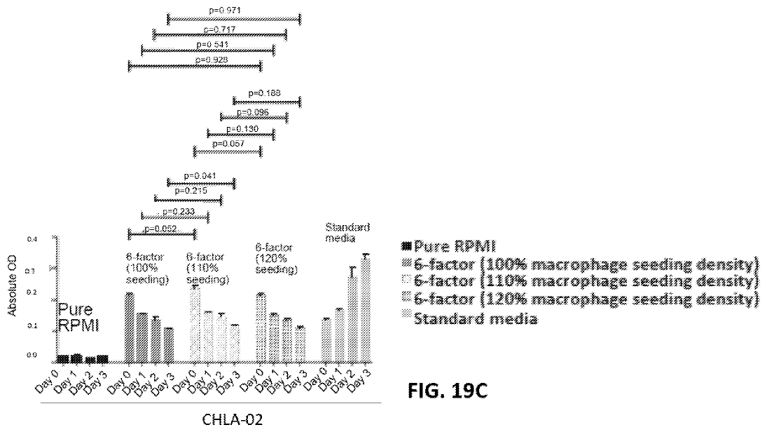

To elucidate the effects of the secretome on tumour cell proliferation, the inventor of the present disclosure cultured a panel of 6 individual patient-derived ATRT cell lines in all three in-vitro conditions of macrophage-tumour microenvironment the inventor of the present disclosure have developed (FIG. 2B): Condition 1—Tumour cell factors, Condition 2—CD11b+ cell factors, Condition 3—CD11b+ and tumour cell co-culture factors. FIG. 4A to 4H show tumour cells that proliferate and rearrange to form extensive, interlacing, web-like structures connecting cells over long-distance radius in macrophage secretome during nutrient stress. The inventor of the present disclosure contrasted the growth of the 6 tumour cell lines (FIG. 4A to 4B) in all 3 microenvironment conditions (using supernatants derived from respective cells cultured in RPMI base media without any serum nor growth factors) against pure RPMI base media (control, without any serum nor growth factors), over a duration of 72 h. Clearly, pure RPMI base media (control, without any serum nor growth factors) was a state of nutrient stress and was not able to support tumour cell proliferation (FIG. 4A to 4B). Supernatants derived from CD11b+ cell factors (FIG. 4A) and CD11b+ and tumour cell co-culture factors (FIG. 4B), resulted in higher tumour cell proliferation compared to tumour cell factors (FIG. 18A). Among all 6 ATRT cell lines, 2 cell lines (CHLA-02 and BT-37) displayed an obvious increase in optical density (OD) readings above the baseline OD readings of secretome (FIG. 19). BT-37 was a patient-derived xenograft cell line of ATRT. This may suggest certain tumour cell lines can preserve intrinsic capability to respond to macrophage-related factors. It was interesting to note that the 2 cell lines which demonstrated higher proliferative abilities (CHLA-02, BT-37) were the same cell lines that were marginally secretome-rich compared to all other cell lines (secretome-bland, FIG. 2C). This suggests the notion that through in-vitro passaging, most tumour cell lines possibly lose components of microenvironment-responsive factors/receptors in the stroma, as suggested by the lesser growth-inducing response stimulated by exogenous secretome in the other 4/6 cell ATRT lines. The growth-promoting effects of these supernatants peaked at 72 h, beyond which tumour proliferation rate trended down (FIGS. 4B, 4C and 4D).

These results further indicate that the panel of cytokine-chemokine factors alone were not sufficient to sustain long term tumour growth in-vitro beyond 72 h without standard growth media supplements (standard in-vitro nutrients also need to be refreshed every 48-72 h for standard cell cultures of these cell lines). This suggests that such macrophage-tumour micro-ecosystems possibly provide early and temporary niches for initial tumour growth spurt, and an alternative fuel source during nutrient-stress state when the microenvironment is deprived of nutrients. Supernatants derived from CD11b+ and tumour cell co-culture, provided the best sustained growth promoting effect at 72 h, compared to CD11b+ supernatants (FIG. 2B, FIG. 4). Tumour-cell-derived conditioned media was not effective in sustaining tumour growth (FIG. 18A), indicating tumour cell factors were not sufficient (FIG. 2B). Six factors in combination were effective. Cytokine array results earlier indicated supernatants derived from CD11b+ and tumour cell co-culture factors contained higher quantities of these six specific cytokines-chemokines compared to CD11b+ conditioned media (FIG. 2B). These six factors CCL2/MCP-1, MIP-1α/MIP-1β, CCL5/RANTES, MIF, IL-1ra/ILIF3, IL-8 have been independently described to affect macrophages or tumourigenesis.

Furthermore, for the first time, the inventor of the present disclosure observed that the unique combination of these 6 factors was capable of preventing tumour cell death in all four ATRT cell lines tested (CHLA-02, BT-37, CHLA-04, CHLA-06, FIG. 4E). This secretome was comparable to standard growth media in preventing cancer cell death up to 48 h, remarkably sustaining a critical mass of cancer cells during nutrient stress. At longer timepoints 72-96 h, more cell death occurred in secretome compared to standard growth media (FIG. 4E), indicating that secretome was able to provide initial support, but secretome alone without nutrient supply was insufficient for prolonged periods. It is particularly interesting to note that secretome was able to prevent cell death for much longer duration of 72-96 h in CHLA-04, which was derived from a recurrent ATRT tumour obtained at autopsy with widespread dissemination to the brain and cerebrospinal fluid (FIG. 4E). This indicates that in this highly aggressive tumour model, although the secretome did not cause a high degree of tumour proliferation (FIGS. 4A, 4B), instead it was capable of preventing tumour cell death for longer periods, contributing to a mechanism for tumour to survive and disseminate. A similar ability of secretome in preventing cell death for prolonged periods 72-96 h (FIG. 4E) but not support a high degree of tumour proliferation (FIGS. 4A, 4B), was observed in CHLA-06, the most aggressive and fast growing ATRT cell line in-vitro. These findings in the most aggressive cell lines-the fastest growing in-vitro (CHLA-06), as well as from a recurrent tumour derived at autopsy with widespread dissemination to the brain and cerebrospinal fluid (CHLA-04), suggest a possible mechanism for the secretome in tumour resistance and relapse.

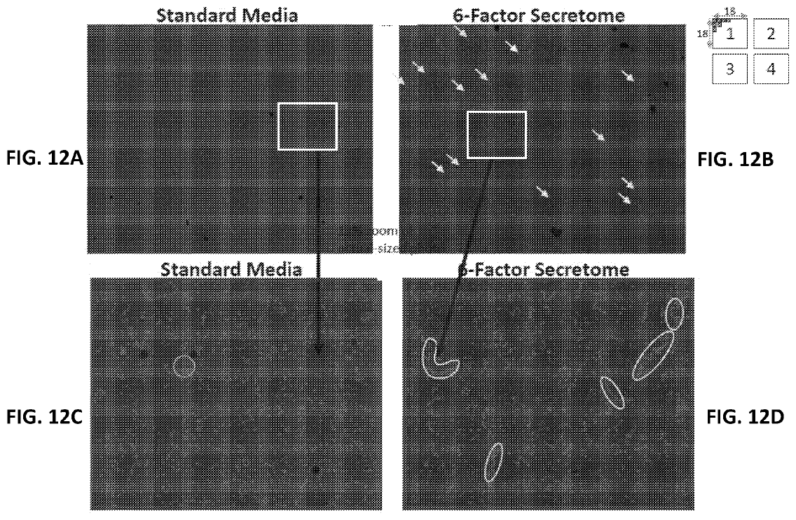

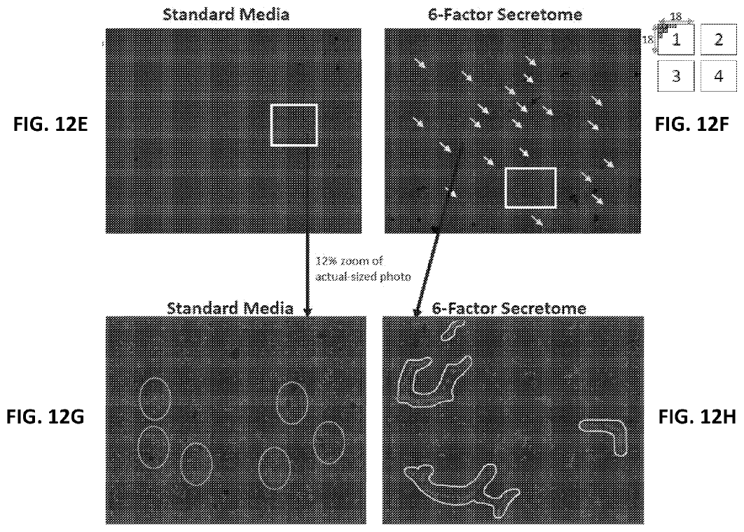

The inventor of the present disclosure discovered that tumour cells which normally grow in neurospheres in standard growth media, when instead grown in secretome, rearrange themselves to form interlacing web-like structures connecting cells linearly over long-distance radius in-vitro (linear patterning), after 24 hours of nutrient stress (FIGS. 4F, 4G, FIG. 20 to 24). Cancer cells on in-vitro cultures have never been shown to exhibit such a capability in standard media conditions. This observation suggests a crosstalk between cells in a secretome microenvironment during nutrient stress or deprivation, which can be important for tumour cells to survive, re-arrange themselves into a web-like architecture to remain resilient without a stable and continuous nutrient supply. FIG. 12A to FIG. 12H show large image photomicrographs that capture large areas of tumour cells (CHLA-02) grown in T75 flask demonstrating the distinct interlacing web-like architecture of tumour cells grown secretome which contrasted with tumour cell clusters growing in standard growth media (control).

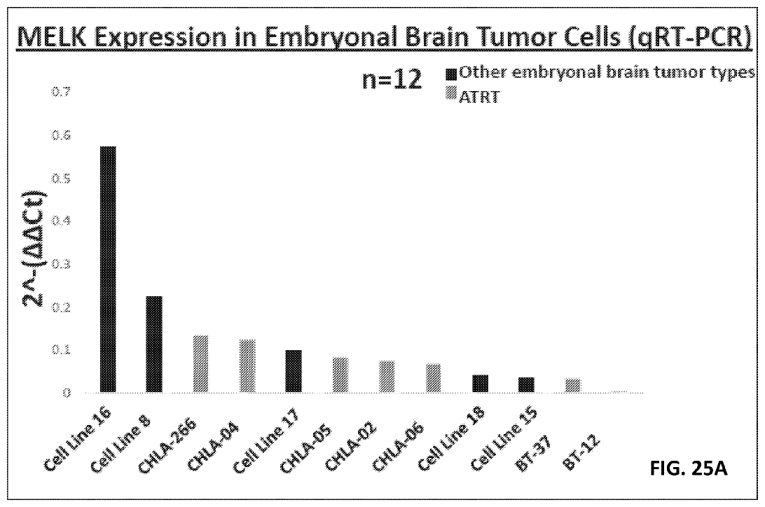

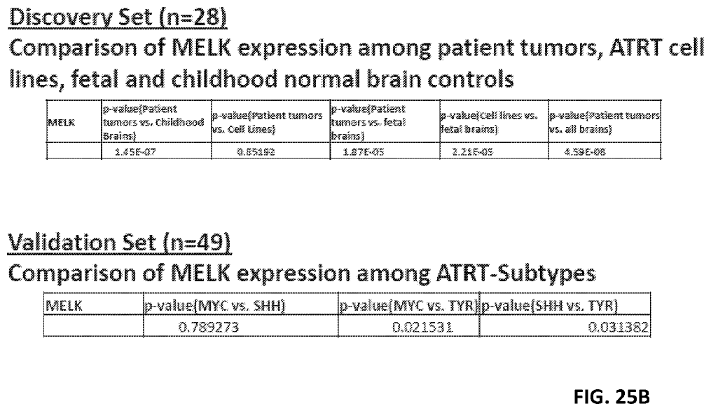

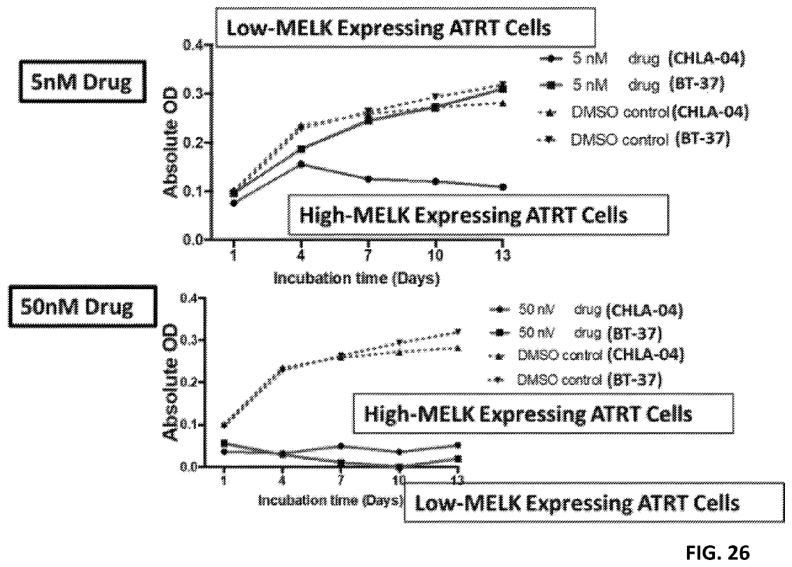

Next, to evaluate the effects of secretome in modulating drug response within the tumour compartment, the inventor of the present disclosure curated potential cell cycle targets within ATRT patient tumours and available corresponding molecular inhibitors. The inventor of the present disclosure postulated that the tumour compartment will be enriched in cell cycle genes during rapid tumour growth and cell division, and therefore cell cycle genes would provide an appropriate target to inhibit growth of the tumour compartment. The inventor of the present disclosure and other groups found maternal embryonic leucine kinase (MELK) enriched among patient ATRT tumours, as well as other tumour types. FIG. 5A to 5H show that secretome impedes drug response and provides a protective niche to rescue tumour cells from initial drug insults. The inventor of the present disclosure further established MELK as a universal target across three ATRT-subtypes (FIG. 5A). MELK was expressed among 6/7 ATRT cell lines (FIG. 25, BT-37 lower, ATRT-95 no expression). OTSSP167, a molecular inhibitor of MELK, was efficacious across a panel of six ATRT cell lines in nanomolar ranges (FIG. 5B, FIG. 26). OTSSP167 induced apoptotic cell death of ATRT cells (CHLA-02, BT-37), causing apoptotic cell death (58.1% early and late apoptotic cells in BT-37 cells, 50.7% in CHLA-02 cells at 48 hours of OTSSP167 treatment, FIG. 5C). Immunoblotting demonstrated p-Histone H 2A.X was increased after 24 h, 48 h and 72 h OTSSP167 treatment in CHLA-02 and BT-37 cells (FIG. 5D), indicative of OTSSP167 inducing DNA damage. c-PARP was also increased with OTSSP167 treatment (FIG. 5D), supporting activation of intrinsic apoptosis pathway. p53 was increased with OTSSP167 treatment, consistent with normal stress response. The inventor of the present disclosure selected the two cell lines CHLA-02 and BT-37 which demonstrated stronger growth proliferative capabilities in the secretome (FIG. 4), to examine for drug response in the presence of secretome.

The inventor of the present disclosure found that the presence of secretome reduced drug efficacy during initial phase (FIG. 5E), providing a pro-survival niche to rescue tumour cells during early drug response. The inventor of the present disclosure show that tumour cells cultured in standard growth media with OTSSP167, exhibited rapid, sustained inhibition of tumour growth indicating a rapidly efficacious drug response in standard growth media (CHLA-02, BT-37, FIG. 5E). In contrast, tumour cells cultured in secretome derived from CD11b+ cells conditioned media or supernatants derived from CD11b+ and tumour cell co-culture factors, consistently demonstrated a dampened response to tumour growth inhibition by OTSSP167 in secretome microenvironment (FIG. 5E). Six factors in combination were effective in dampening the drug response, indicating the drug is less effective in a secretome microenvironment compared to an in-vitro screen employing standard growth media. Within patient tumours, these 6 factors were enriched in the microenvironment, which suggests a lower drug efficacy in a secretome-rich microenvironment in patient tumours.

The secretome is a scalable and clinically relevant resource as a laboratory-stage phenotypic screen for drug testing among cancer cell lines. It can predict the pro-survival contributory component of a restricted repertoire of microenvironment cytokines-chemokines. It serves as a tool to measure the drug efficacy in the presence of a minimal pool of microenvironmental factors. In addition, these factors are stable during long term storage at −80° C. and maintained its efficacy after long-term storage at −80° C. for 3 years (FIG. 4D—results of CHLA-02 (Replicate 1) and BT-37 assays (Replicate 1) using supernatants stored for 3 years at −80° C.). The six-factor-secretome drug screening approach of the present disclosure, can provide a readily available scalable resource for in-vitro laboratory testing of other cancer types to identify cancers that exploit secretome as alternative fuel source. This secretome provides a favourable tumour ecosystem that reprograms malignant cells to survive nutrient stress and other selection pressures such as early drug response. Additionally, when treated with OTSSP167, tumour cells maintained their interlacing architecture in secretome (FIGS. 5H, 5E, FIG. 27, FIG. 28), in contrast to tumour cells (CHLA-02) breaking up into smaller clusters when treated with OTSSP167 in standard growth media, suggesting a potential mechanism for therapy resistance provided by secretome.

These findings indicate a pattern of pro-survival niches supported by secretome, which is advantageous for tumour cell survival during nutrient stress and exogenous drug stress. In a native, no drug state, media nutrients induced a slow gain in proliferative rate, while a six-factor-secretome microenvironment in a nutrient-poor cancer cell niche provoked a rapid and sustained proliferative rate (FIG. 4). This suggests that the secretome provides a superior ecosystem for tumour growth spurt during nutrient deprivation. In a drug-exposed state, tumour proliferative response to OTSSP167 was dampened in the presence of secretome (FIG. 5E), in contrast to standard media nutrient environment whereby drug exposure resulted in rapid inhibition of tumour proliferation. This indicates that a drug which is shown efficacious in-vitro in standard growth media (traditional drug testing approach), when tested on the same cell lines in a secretome-screen, it is less efficacious.

This quadruple mechanism of secretome capable of (1) inducing rapid early growth spurt and (2) preventing tumour cell death during nutrient stress, simultaneously (3) dampening drug response of tumour cells and maintaining stable interlacing web-like architecture of tumour cells when exposed to drug, provide strong advantages for tumour cell resilience and therapy resistance (FIG. 4G). Some patient tumours are secretome-rich (FIG. 2C to 2D), which highlights a gap during translation of in-vitro effective drugs to clinical trials, whereby there is an unmet need to better accurately match patient tumours which are secretome-rich and potentially less responsive to certain drugs.

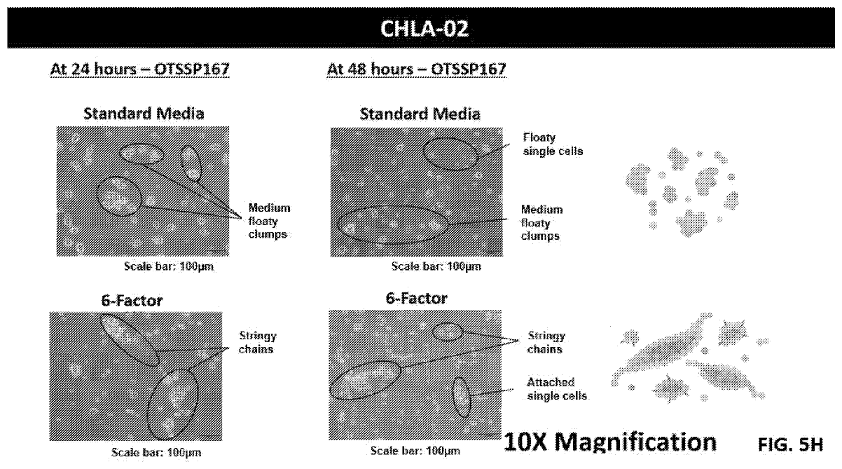

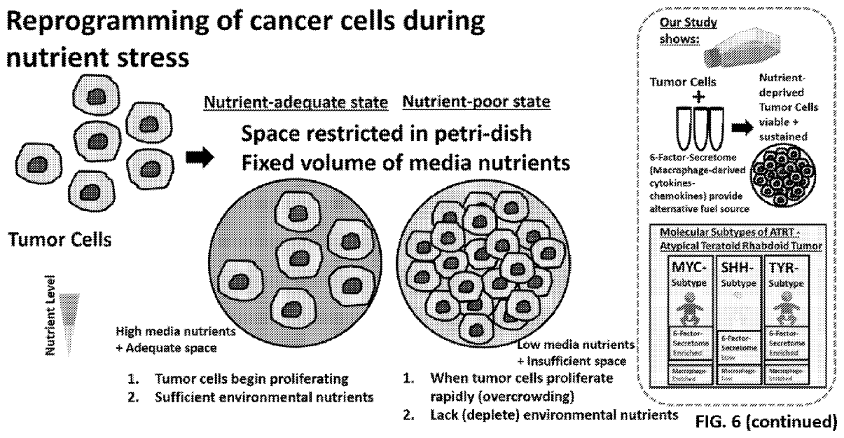

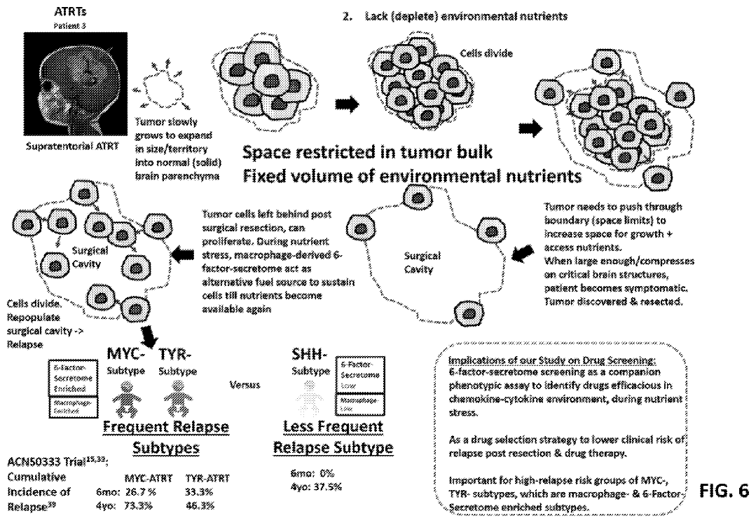

Another consideration is that macrophages can provide these secretome micro-ecosystems to promote tumour relapse-an alternative fuel source to protect against cell death (FIGS. 4E, 4F) and accelerate tumour growth spurt (FIG. 4D) of residual cancer cells residing near tumour margins post-surgical bulk resection of tumour-a mechanism for tumour relapse. The inventor of the present disclosure has observed macrophage-enriched compartment with high density of macrophages nested at tumour borders in some patient tumours resected (FIG. 1A). This observation is further strengthened by the finding that frequent relapse-subtypes of ATRT-MYC-ATRTs, TYR-ATRTs (FIG. 6) are also macrophage-enriched and secretome-rich subtypes (FIG. 2C-2D). Data from ACNS0333 reported patients with SHH-ATRTs had no relapses in the first 6 months and a cumulative incidence (CI) of relapse of 37.5% at 4 years, while for TYR-ATRTs and MYC-ATRTs had CI of relapse of 33.3% and 26.7% at 6 months, and 46.3% and 73.3% at 4 years respectively (p=0.088, Presented. Unpublished data.). This trend is interesting, despite the fact that ACNS0333 was not powered to determine prognostic indicators of relapse. Larger data set compilation is needed to study these variables.

A limitation of the study of the present disclosure was that only six-factors (identified out of 36 candidates on cytokine array) were employed in the in-vitro assays of the present disclosure. The threshold for detection was restricted by the minimum quantity of protein detectable by cytokine arrays. The serum-free/cell-free supernatants (FIG. 2B) could contain other factors in lower quantities not evaluable through this assay. Using a higher specification protein assay such as mass spectrometry may detect a greater spectrum of chemokines-cytokines. However, the data of the present disclosure has proven that patient tumours, particularly MYC-ATRTs and TYR-ATRTs are highly enriched in 36 candidates on cytokine array (FIG. 2B-2C), likely harbouring more than 36 cytokines-chemokines. Merely defining all the cytokines-chemokines using high resolution protein assays adds little knowledge to the mechanistic functions of the entire secretome spectrum in the patient tumour. More importantly, the inventor of the present disclosure shows that using this culture system comprising of a minimum of 6 factors, the inventor of the present disclosure were already able to elicit the effects on tumour growth spurt and dampening of drug efficacy. Another consideration is that the secretome assay of the present disclosure measures up to 72 h. The amount of protein produced by CD11b+ cells in the in-vitro assay of the present disclosure was in physiological quantities, and not unlimited. Hence, this assay also parallels the biological concentrations of endogenous secretome production in patient tumours, which the data of the present disclosure recapitulated—a small quantity of this mixture of cytokines-chemokines in a patient tumour is sufficient to support an initial growth spurt which is critical during tumour initiation. Within patients' tumours, the problem is amplified because macrophages residing in-situ can continuously produce secretomes as alternative fuel source to sustain a minimum pool of critical cancer cells during nutrient stress until nutrient sources replete through new tumour vasculature. This is important in solid tumours—accessibility to nutrients within solid tumours is regulated by proximity to the vasculature. The growth and survival of rapidly expanding solid tumours require a continuous oxygen and nutrient supply which needs to be maintained by the tumour neovasculature. Cells located adjacent to the vasculature use nutrients and oxygen to fuel proliferation. However, cells distant from the vasculature have diminished accessibility to nutrients and will need to engage alternative fuel resources such as macrophage secretomes. Therefore, drugs which are efficacious both in nutrient-replete and secretome-based conditions will be highly attractive.

Macrophage infiltration in ATRTs was reported independently in other smaller data sets, with subtype-specific differences-previous analysis however, reflected infiltration of CD68+microglia/macrophages were significantly higher in SHH-ATRTs (16 tumours) compared to both TYR-ATRTs (10 tumours, p=0.0006) and MYC-ATRTs (8 tumours, p=0.0157), which is in contrast to the current findings of the present disclosure which uncovered SHH-ATRTs comprise of a macrophage-depleted tumour-subtype. This also strengthens the challenge the inventor of the present disclosure highlighted earlier-when studying macrophages in patient tumours, specimens are harvested and immortalized at a single time-point. During this process, the inventor of the present disclosure loses the opportunity to study the real-time dynamics of macrophage and tumour compartments in-situ as an ecosystem. Although the evaluation of CD163 as a marker for M2 macrophages may not be fully elucidated, numerous other studies have consistently used CD163 for that purpose, including studying tumours and cell lines. In addition, tumour-infiltrating macrophages in the brain comprise of both resident microglial and bone-marrow-derived circulating monocytes entering the brain. There continues to be debate about the existence of M1 and M2 microglial. The mechanistic approach of the present disclosure of employing the secretome assay enabled a multi-time-point sampling to study the milieu during tumour development.

From the data of the present disclosure on BT-37 (FIG. 5E), this xenograft-derived cell line supernatant alone can support some tumour growth unlike the other cell line (CHLA-02), suggesting the likelihood of growth-promoting factors contributed by mouse stroma components in xenograft cell lines (BT-37), which may be absent in patient-derived-cell lines (CHLA-02) after long term passaging. In-vivo, OTSSP167 was effective in prolonging animal survival on an aggressive autopsy-derived patient-derived orthotopic xenograft mouse model of ATRT (FIG. 5F). But like in most preclinical testing, the mice eventually succumbed to the tumour even though drug administered was able to slow tumour growth. This is a gap for clinical translation. Microenvironmental factors constantly secreted by other cell types in the host tissue, can provide a critical pro-survival niche for early tumour cells. Tumour cells can be sustained even in the absence of environmental nutrients, which circles into the important challenge of tumour relapses. The assays of the present disclosure demonstrate—as long as there are supporting cell types (macrophages) providing a minimal secretome, in a niche with a few surviving tumour cells, such as in conditions after surgical bulk resection/post-therapy, tumour cells can be re-invigorated (FIG. 6). Since drug exposure times for invitro assays can differ markedly from those achievable in-vivo, the in-vivo data of the present disclosure conclusively demonstrated that the therapeutic activity of OTSSP167 can induce tumour growth reduction at OTSSP167 levels achieved in-vivo. Our results indicate OTSSP167 was clinically effective in-vivo on the aggressive autopsy-derived ATRT Model (p=0.032, FIG. 5F) of the present disclosure. OTSSP167 has been shown to penetrate blood-brain-barrier of another ATRT mouse model. Taken together, the data of the present disclosure reveals for the first time, the functional hallmarks of secretome in macrophage-tumour ecosystems, which can overcome nutrient deprivation states by providing a constant pro-survival niche. Such a secretome can re-invigorate a small but critical, surviving pool of tumour cells, which can be important mechanisms of relapse and treatment resistance.

The study of the present disclosure addresses the knowledge gap on how a macrophage-associated secretome, enriching the microenvironment of patient tumours, can mechanistically impact tumour proliferation during nutrient stress by providing an alternative fuel source. Further, these findings raise further questions on how such macrophage-associated secretomes can impact the clinical success of drugs during clinical trial translation, since drugs are traditionally tested using standard media in-vitro, but patient tumours can be secretome-rich. The inventor of the present disclosure proposes a secretome-screen as a companion phenotypic assay to identify drugs efficacious in chemokine-cytokine environment to evaluate the contributory component of tumour microenvironment factors, in conjunction with traditional in-vitro drug screening employing standard growth media, as well as in-vivo preclinical testing. The approach of the present disclosure adds value to the strategy for drug selection and patient stratification, to lower clinical risk of relapse post-resection and post-drug therapy. This is particularly important for high-relapse risk groups of MYC-and TYR-subtypes, which are macrophage-and six-factor-secretome enriched subtypes.

In summary, the inventor of the present disclosure found that a secretome derived from macrophages, enables cancer cells to proliferate in a nutrient-poor state within a cancer cell niche of a paediatric embryonal brain tumour, ATRT, during early tumour development. This secretome is equal to standard growth media in preventing cancer cell death, remarkably sustaining a critical mass of cancer cells during nutrient stress. Specific molecular subtypes of ATRT emerged as secretome-enriched, matching macrophage-enrichment patterns. High-relapse-risk subtypes were discovered to be concurrently secretome-rich and macrophage-enriched. The results of the present disclosure unravel for the first time, a previously unexplored role of a unique macrophage secretome in providing an alternative fuel to sustain cancer cells when nutrients are depleted, and implications in relapse-subtypes. Furthermore, the response of ATRT cells to drugs is altered in secretome. Clearly, the presence of the secretome reduces OTSSP167 efficacy and protects against cancer cell death during initial tumourigenesis, providing a pro-survival niche to rescue drugged cells during early drug exposure. Drugs effective against cancer cells screened in standard media, may not be equally efficacious in secretome-phenotypes of patient tumours. Strikingly, secretome prevents tumour cell death for prolonged periods in aggressive tumour models and models of cerebrospinal dissemination, suggesting a role in tumour resistance and relapse. The inventor of the present disclosure discovered that tumour cells which normally grow in neurospheres in standard growth media, when instead grown in the secretome, rearrange themselves to form interlacing web-like architecture connecting cells linearly over long-distance radius in-vitro, after 24 hours. This observation suggests a crosstalk between cells in a secretome microenvironment during nutrient stress or deprivation, which can be important for tumour cells to survive and remain resilient without a stable and continuous nutrient supply. Additionally, when treated with OTSSP167, tumour cells maintained these interlacing structures in secretome, which suggests a potential mechanism for drug resistance. Therefore, the secretome of the present disclosure can be used to identify drug resistance in cells in response to drug treatment.

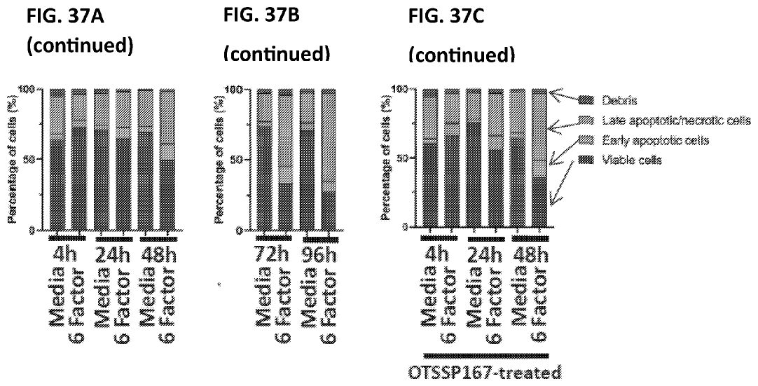

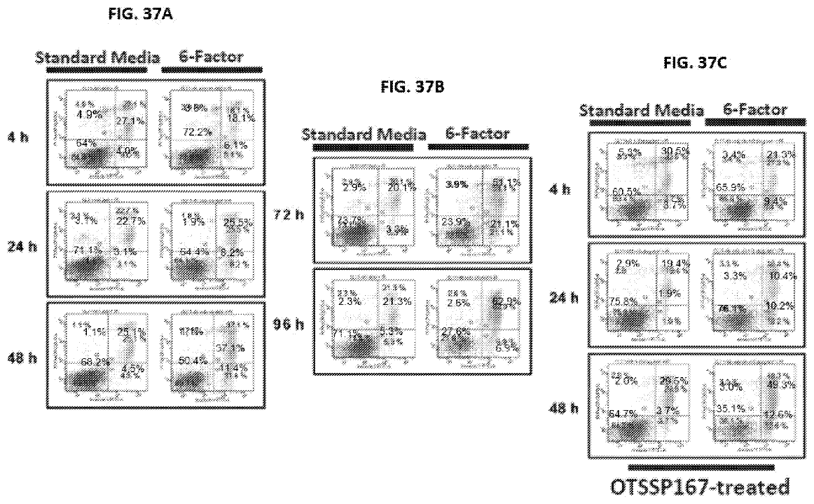

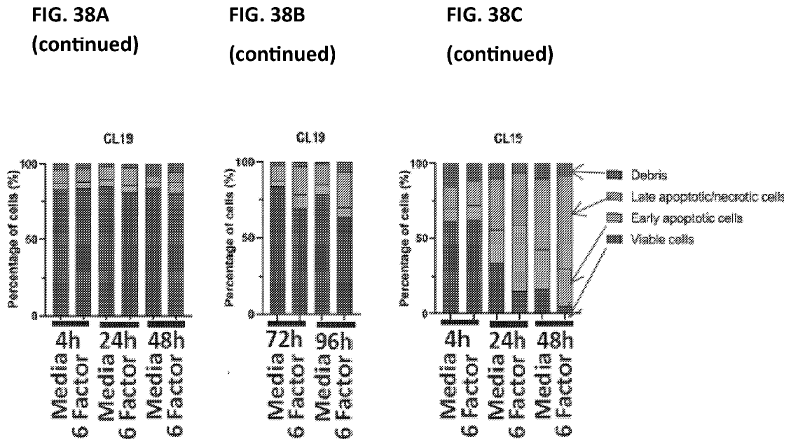

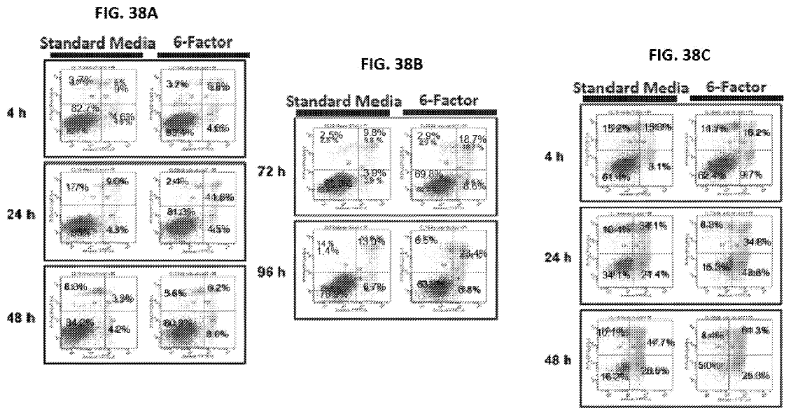

The drug response for a diseased cell (such as medulloblastoma cells) may be more sensitive in the presence of the secretome as compared to standard media. In response to OTSSP167, neurospheres CHLA01 (primary tumour cells) and CHLA01R (recurrent/relapse tumour cells) survive better while Dao7 and D341 medulloblastoma cells have higher cell death in secretome compared to standard media. In response to Ispinesib, D341, CHLA-01 and CHLA-01R survive better in secretome compared to standard media. In addition, FIG. 37A to 37C shows CHLA-02 cells undergoing more cell death with OTSSP167 in secretome compared to standard media conditions. FIG. 38A to 38C shows BT-37 cells undergo more cell death with OTSSP167 in secretome compared to standard media conditions.

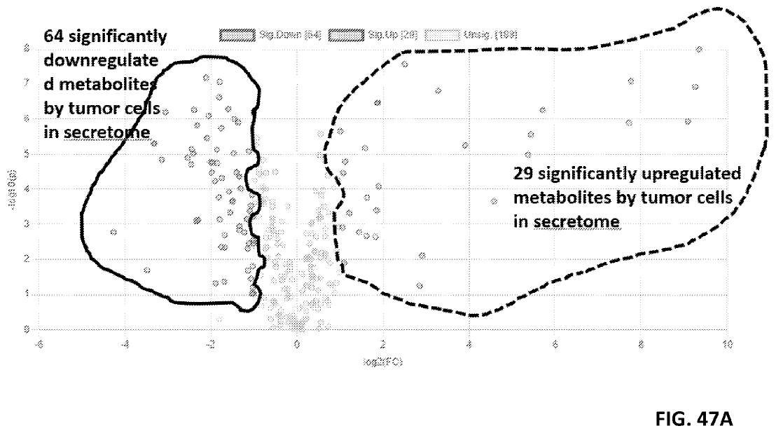

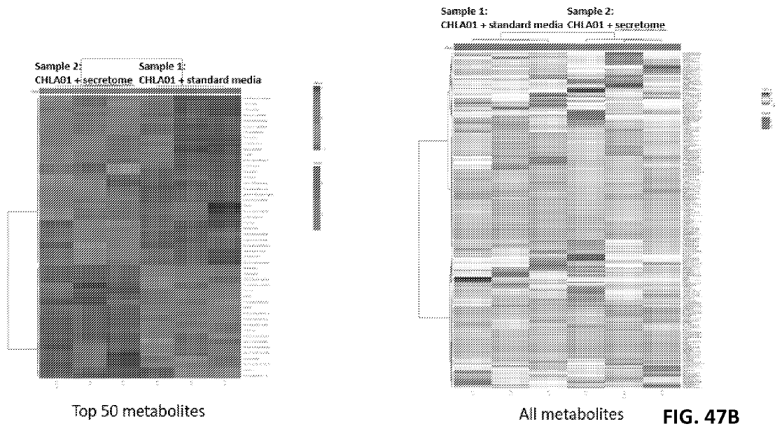

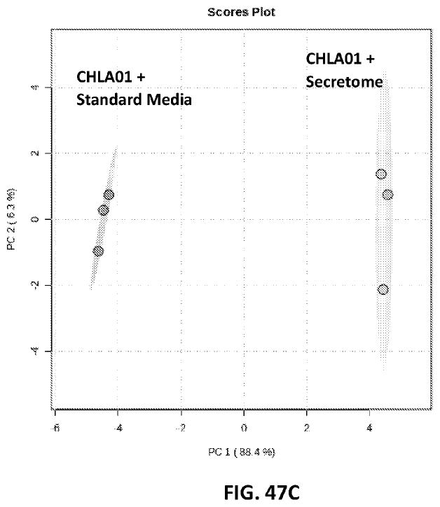

The present disclosure shows summary of metabolomic studies with untargeted Orbitrap assay (FIG. 47 to FIG. 50) Data were analysed using MetaboAnalyst 6.0. software. Sample 1 includes CHLA01 grown in standard media and sample 2 includes CHLA01 grown in secretome. Three replicates were performed with CHLA01 grown in standard media versus three replicates with CHLA01 grown in secretome (total of six experiments). Part 1 analyses includes using the entire list (all) of metabolites that emerged from the above six experiments (without expression values), using pathway metabolite set libraries (1) SMPDB-The Small Molecule Pathway Database)(99 metabolite sets based on normal human metabolic pathways). Top 25 Enriched Metabolite Sets demonstrated that CHLA01 tumour cells cultured in both secretome and standard media, are enriched in methylhistidine metabolism. Part 2 analyses includes using Upregulated or Downregulated metabolites detected in tumour cells (CHLA01) cultured in secretome for 24 h (compared against tumour cells cultured in standard media for 24 h), generated in the experiment (3 replicates for each of both samples) of the present disclosure, using pathway metabolite set libraries (1) SMPDB-The Small Molecule Pathway Database) (99 metabolite sets based on normal human metabolic pathways), (2) KEGG (80 metabolite sets based on KEGG human metabolic pathways (Dec 2023)), and (3) Drug related library (461 metabolite sets based on drug pathways from SMPDB).

Pathways that are upregulated with the SMPDB Database includes biotin metabolism (Top #1 pathway with enrichment ratio<25), taurine & hypotaurine metabolism (Top #2 pathway with enrichment ratio<15) and ammonia recycling pathway (enrichment Ratio<10). Pathways that are downregulated with SMPDB Database includes methylhistidine metabolism (Enrichment ratio<12). Pathways that are upregulated with KEGG Database includes taurine & hypotaurine metabolism (Top #1 pathway with enrichment ratio<25) and biotin metabolism (Top #2 pathway with enrichment ratio<20). Pathways that are downregulated with KEGG Database includes valine, leucine, isoleucine biosythesis (top #1 pathway with enrichment ratio<25), phenylalanine, tyrosine, tryptophan biosynthesis (top #1 pathway with enrichment ratio<25). There are drug pathways that are upregulated from SMPDB database with pathways that emerged including amikacin pathway.

Therefore, important metabolic pathways that emerged from the metabolomic studies include Methylhistidine metabolism, Biotin metabolism, Taurine and HypoTaurine Metabolism and others. Methylhistidine metabolism emerges as an important metabolic pathway when the list of metabolites from both tumour cells (CHLA01) grown in both secretome & standard media, are combined. Methylhistidine is known to be implicated in cancer cachexia which suggests that secretome reprograms cancer cell metabolism. In actual patient tumour context, the secretome microenvironment possibly generates metabolites that promote weight loss in cancer patients (cancer cachexia) by altering the metabolism/nutrient use. Cancer drug screening in-vitro, if able to identify drugs that downregulates methylhistidine metabolism in cancer cells (in secretome microenvironment), will be able to not just slow down the cancer progression in the patient but also avoid the patient suffering from cancer cachexia. If cancer drug screening is done purely on standard media, the metabolites that are altered in secretome (such as methylhistidine) will be missed. Therefore, secretome as a companion drug development tool, will advantageously provide this additional important parameter of identifying effective drugs that may avoid cancer cachexia in patients. The data of the present disclosure shows that Methylhistidine metabolism (Enrichment ratio<12) is downregulated in tumour cells (CHLA01) grown in secretome compared to standard media. This can indicate that cancer cells in the state of nutrient deprivation (in cachexic patients with low muscle reserves) can switch to secretome for sustenance and continue the growth of the tumour.

Biotin metabolism is enriched in tumour cells (CHLA01) grown in secretome compared to standard media. Biotin has been established to be higher in tumour compared to non-tumour tissues. Taurine and HypoTaurine Metabolism is enriched in tumour cells (CHLA01) grown in secretome compared to standard media. This pathway has been implicated in gliomas and pediatric brain tumours. Targeting drugs effective in secretome become an important and attractive strategy.

### Important Translational/Clinical Implications From Data of Metabolomic Studies

### A. Methylhistidine Metabolism Emerges as an Important Metabolic Pathway When the List of Metabolites From Both Tumour Cells (CHLA01) Grown in Both Secretome & Standard Media are Combined.

Methylhistidine is known to be implicated in cancer cachexia which suggests that the secretome reprograms cancer cell metabolism. In actual patient tumour context, the secretome microenvironment possibly generates metabolites that promote weight loss in cancer patient (cancer cachexia) by altering the metabolism/nutrient use. Cancer drug screening in-vitro, if able to identify drugs that downregulates methylhistidine metabolism in cancer cells (in secretome microenvironment), will be able to not just slow down the cancer progression in the patient but also avoid the patient suffering from cancer cachexia. If cancer drug screening is done purely on standard media, the metabolites that are altered in secretome (such as methylhistidine) will be missed. Therefore, secretome as a companion drug development tool, will advantageously provide this additional important parameter of identifying effective drugs that may avoid cancer cachexia in patients. The data of the present disclosure shows that Methylhistidine metabolism (Enrichment ratio<12) is downregulated in tumour cells (CHLA01) grown in secretome compared to standard media. This can indicate that cancer cells in the state of nutrient deprivation (in cachexic patients with low muscle reserves) can switch to secretome for sustenance and continue the growth of the tumour. Targeting drugs effective in secretome thus becomes an important and attractive strategy.

B. Biotin Metabolism is Enriched in Tumour Cells (CHLA01) Grown in Secretome Compared to Standard Media. Biotin has Been Established to be Higher in Tumour Compared to Non-Tumour Tissues, Which Correlated to Overexpression of Biotin Receptors

Cancer cells appear to have hijacked biotin metabolism to sustain their proliferation. These observations have also motivated efforts to develop biotin-based drug delivery systems for cancer treatment. Enriched biotin metabolism in CHLA01 grown in secretome also indicate reinforced and enhanced capabilities of cancer cells to survive and proliferate in secretome (and secretome-phenotypes of patient tumours) as shown in the present disclosure.

Biotin is a water-soluble vitamin required by all organisms because of its essential role in carboxylation reaction. In non-cancerous cells, a major function of biotin is to serve as a coenzyme for biotin-dependent carboxylases, including acetyl-coenzyme A (CoA) carboxylase 1 (ACC1), ACC2, methylcrotonoyICoA carboxylase 1 (MCCC1), propionyl-CoA carboxylase (PCC), and pyruvate carboxylase (PC), which play crucial roles in fatty acid metabolism, gluconeogenesis, and amino acid catabolismthese carboxylases are synthesized in an inactive form (apocarboxylases) and require activation by the holocarboxylase synthetase (HLCS) enzyme via biotinylation. Subsequently, biotin must be released from the holocarboxylases, which is catalyzed by the enzyme biotinidase. Although less well understood, histone proteins can also be biotinylated by HLCS, and the levels of biotinylated histones may affect cell proliferation, epigenetic, and gene expression alterations. As expected, cancer cells appear to have hijacked biotin metabolism to sustain their proliferation. Biotin levels were higher in tumour versus non-tumour tissues, which correlated with the overexpression of biotin receptors. These observations have also motivated efforts to develop biotin-based drug delivery systems for cancer treatment. Given that regulated biotin metabolism is ubiquitous and essential across nature, targeting biotin regulation within deregulated pathways may represent a potential approach to disable GBM.

Dependency of GBM on biotin distribution suggests that the rational co-targeting of biotin-dependent metabolism and epigenetic pathways may be explored for GSC eradication. Biotin is a water-soluble vitamin required by all organisms by virtue of its essential role in carboxylation reactions. Although the metabolism and role of biotin in intermediary metabolism are well established, biotin remains one of the most poorly understood water-soluble vitamins in terms of nutritional requirements and responsiveness to physiological and pharmacological states. Significant advances in the understanding of biotin nutrition have been recently accomplished through the description of the kinetics and regulation of biotin transport and improved methods for biotin status assessment. Additionally, the potential role of biotin in the regulation of gene expression has been strengthened through description of altered gene expression during biotin deficiency and through newly described enzymatic activities of the enzyme biotinidase.

C. Taurine and Hypo Taurine Metabolism is Enriched in Tumour Cells (CHLA01) Grown in Secretome Compared to Standard Media. This Pathway has Been Implicated in Gliomas and Pediatric Brain Tumours.

### Cryopreservation of Cells

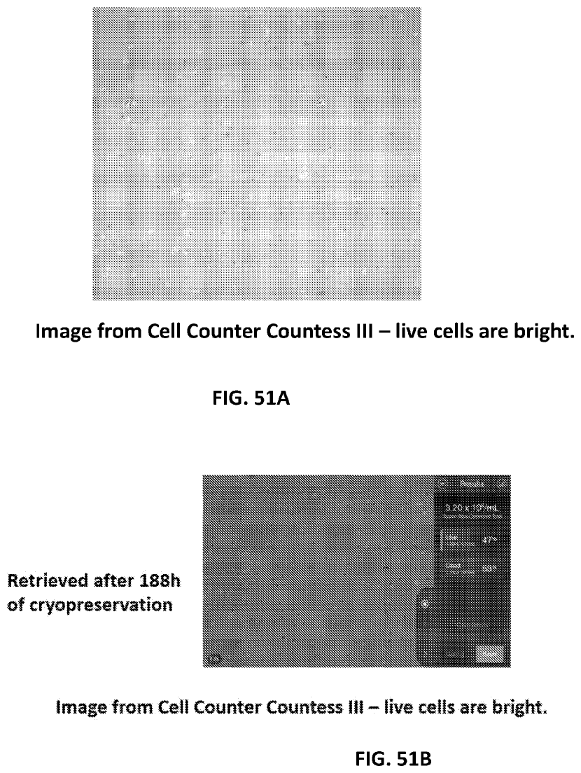

The inventor of the present disclosure discovered that the secretome serves the additional function for cell cryopreservation (FIG. 51), a novel substitute, non-inferior or superior to standard media-based (traditional method) method of cell cryopreservation. The inventor of the present disclosure made freezing media using 90% of secretome and 10% DMSO. CHLA 01R cells were frozen using freezing media made of 90% 6-factor secretome and 10% DMSO. The inventor of the present disclosure cryo-froze 2.49 million live cells (63% viability) in 1.9 ml of freezing media made of 90% of secretome and 10% DMSO, in a cryovial, placed in Mr Frosty in −80 degrees Celsius and then transferred to liquid nitrogen within 24 h, which is the standard protocol for cryopreservation. The inventor of the present disclosure retrieved the cells 65 hours later, and the total cell count was slightly higher than what was frozen, 2.7 million cells (51% viability). A loss of ~10% viability is similar if not superior to the results using standard media-based freezing media in our experience with CHLA01R cells. In addition, cells were retrieved 188 hours later, and the total cell count was higher than what was frozen, 3.0 million cells (47% viability). A loss of ~15% viability is similar if not superior to the results using standard media-based freezing media in our experience with CHLA01R cells. Interestingly, the cells seemed to have been able to maintain or even regain viability possibly during the dispensing processing (room temperature)/the freezing process using secretome-based freezing media, which is usually not the case for standard-media based freezing media. Usually, the total number of viable cells would have decreased after undergoing the freeze-thaw-retrieval harsh cryopreservation process. Therefore, freezing media made of 90% of secretome and 10% DMSO, is comparable if not superior to freezing media made of standard media and 10% DMSO.

Conceptual Advance/Clinical Relevance: The strategy of the present disclosure has important clinical implications. Many preclinical successes have not been equally paralleled by success at clinical trials. This novel macrophage secretome provides a superior ecosystem with an alternative fuel source for tumour growth spurt during nutrient stress. The inventor of the present disclosure discovered that a drug which is shown efficacious in-vitro in standard growth media (traditional drug testing approach), when tested on the same cell lines in a secretome-screen, is less efficacious. Some patient tumours are secretome-rich, while in-vitro drug testing conditions are nutrient-rich and secretome-poor. This highlights a gap during translation of invitro effective drugs to clinical trials, whereby there is an unmet need to better accurately match patient tumours which are secretome-rich and potentially less responsive to certain drugs. This problem is amplified in solid tumours-the growth and survival of rapidly expanding solid tumours require a continuous oxygen and nutrient supply which needs to be maintained by the tumour neovasculature. When such nutrient supplies are lacking, the presence of a macrophage secretome can provide the needed milieu as an immediate alternative fuel source to rescue and sustain tumour cells distant to vascular supply. Tumour cells can therefore survive through interim periods before new nutrients can enrich the microenvironment through newly formed tumour neovasculature. Relapse contributes to a major cause of cancer deaths. The ability of the macrophage secretome to propagate the residual tumour cells during disease relapse, will serve an important mechanism in tumour relapse.

### DETAILED DESCRIPTION OF FIGURES

Example embodiments of the disclosure will be better understood and readily apparent to one of ordinary skill in the art from the following discussions and if applicable, in conjunction with the figures. Example embodiments are not necessarily mutually exclusive as some may be combined with one or more embodiments to form new exemplary embodiments. The example embodiments should not be construed as limiting the scope of the disclosure.

FIG. 1A shows an immunohistochemical staining of CD163 monoclonal antibody on ATRT tumours that demonstrates morphologically, that CD163 is expressed on macrophages and not tumour cells. Macrophage-enriched compartment with high density of macrophages (dark cells stained by CD163 antibody) indicated by arrow, nested at tumour border indicated by arrow. Normal brain (control) is barely infiltrated by macrophages.

FIG. 1B shows intra-tumoural heterogeneity of macrophage (dark cells stained by CD163antibody) infiltration within each single patient tumour (Patient 1-4) demonstrating stroma is distinctly, densely infiltrated by macrophages compared to tumour compartment (p=0.022, Paired T-test). An expanded independent validation cohort demonstrates similar trend of macrophage infiltration by immunohistochemistry in a mixed bag of pediatric brain tumour types (FIG. 13). Macrophages form a distinct perivascular ring suggesting origins related to circulating myeloid cells in Patient 4.

FIG. 1C shows a schematic diagram with strategies to study macrophages infiltrating patient tumours including Strategy 1: Molecular profiling of dissected regions of macrophages in patient tumours, Strategy 2: Single cell sequencing of individual macrophage in patient tumours. Challenges of Strategy 1 include omission of scattered, invasive macrophages in patient tumour, whilst challenges of Strategy 2 include (1) high cost to sequence large numbers of single cells, (2) data limited by the number of cells sequenced (cost limitation), and (3) ensuring good representative population of single cells sequenced. Common problem of both Strategy 1 and 2 is that both strategies involve tearing apart of the macrophage microenvironment compartment from the tumour compartment at a single time-point, resulting in a loss of opportunity to study the in-situ, real-time dynamics between the tumour and macrophage compartments.

FIG. 2A shows a diagram of an in-vitro system to derive six-factor-secretome using CD11b+ human-myeloid-derived cells.

FIG. 2B shows a diagram/blot of the three conditions of macrophage-tumour microenvironment that were established in-vitro: (1) Tumour cell factors, (2) CD11b+ cell factors, (3) CD11b+ and tumour cell co-culture factors. 36-cytokine-chemokine array analyses performed for above conditioned media and co-culture supernatant demonstrated a pattern of 6-cytokine enrichment related to CD 11b+ macrophages in the microenvironment. Presence of tumour cells enhanced the production of specific cytokines-chemokines (CCL2/MCP-1, MIP-1α/MIP-1β,CCL5/RANTES, MIF, IL-1ra/IL-IF3, IL-8) by CD11b+ cells.

FIG. 2C shows a heatmap illustrating six-factor-secretome enrichment in patient tumours in comparison to normal brain tissues and tumour cell lines in Discovery Set of 28 samples (RNA-seq) comprising of 10 patient tumours, 8 patient-derived cell lines and 10 controls (childhood and foetal normal brain tissues). Enrichment pattern of expanded 36-cytokine-chemokine panel (candidates on cytokine array platform) recapitulated six-factor-secretome pattern. According to the heatmap, ATRT patient tumours are upregulated while normal brain controls and ATRT tumour cell lines are downregulated.

FIG. 2D shows a heatmap demonstrating six-factor-secretome enrichment among patient tumours of specific ATRT molecular subtypes (MYC>TYR>SHH) in Validation Set (GSE 70678; Affymetrix U133 plus 2.021) of 49 patient ATRT tumours classified by molecular subtypes. Enrichment pattern of expanded 36-cytokine-chemokine panel (candidates on cytokine array platform) recapitulated six-factor-secretome enrichment pattern among three ATRT molecular subtypes (MYC>TYR>SHH), and was evident among patient ATRT tumours in both FIG. 2C, Discovery and FIG. 2D, Validation Sets. According to the heatmap, MYC-ATRT are highly upregulated, TYR-ATRT are upregulated while SHH-ATRT are downregulated.

FIG. 3A shows dot plots indicating expression of CD163 (M2 macrophages) and CD11b/ITGAM (total macrophages) in Discovery Set (n=28) classified by molecular subtypes.

FIG. 3B shows dot plots indicating expression of CD163 (M2 macrophages) and CD11b/ITGAM (total macrophages) in Validation Set (n=49) classified by molecular subtypes. Patient tumours were enriched in CD11b/ITGAM (pan-macrophage) and CD163 macrophages (M2 macrophage), but not tumour cell lines (Student T-Test. **: p<0.005, NS: not significant). CD11b expression was 112-fold higher (patient tumours: cell lines) and CD163 expression 1083-fold higher among patient tumours compared to cell lines (FIG. 17).

FIG. 3C shows correlation plots of total macrophage marker (CD11b/ITGAM) against marker associated with M2 macrophages (CD163) in patient tumours, patient derived tumour cell lines of ATRT and normal brain controls, in FIG. 3A, Discovery Set and, FIG. 3B, Validation Set. Macrophage markers of various subsets correlated well among patient tumours but not in tumour cell lines or normal brain tissues. Pearson correlation with p-value<0.0.5 and R-square >0.6 show that the two genes are highly positively correlated in gene expression. MYC-ATRTs and TYR-ATRTs were macrophage-enriched tumours, matching six-factor-secretome-enriched subtypes. SHH-ATRTs were macrophage-low and six-factor-secretome-low in enrichment.

FIG. 3D shows graphs that tumours displaying M2 macrophage-enriched phenotype (CD163+) were frequently MYC-ATRTs and TYR-ATRTs.

FIG. 3E shows pathways that tumours with high CD163 expression were significantly enriched in dendritic cell maturation pathway and complement system (positive z-score, see Methods).

FIG. 4A and FIG. 4B show bar graphs depicting growth-promoting effects of each in-vitro microenvironment conditioned media (derived from base media RPMI) on five patient-derived ATRT cell lines (CHLA-04, CHLA-02, CHLA-05, CHLA-06, CHLA-266) and one patient-derived xenograft cell line (BT-37) over Day 0-3, compared to base media RPMI control condition. FIG. 4A show supernatants derived from CD11b+ cells (Microenvironment 1). FIG. 4B show supernatants derived from CD11b+ and tumour cell co-culture (Microenvironment 2). Supernatants derived from tumour cells (Microenvironment 3) shown in FIG. 18. Two cell lines (CHLA-02 and BT-37) demonstrated high proliferative rates in CD11b+ conditioned media and CD11b+ -tumour cell co-culture supernatants, compared to other cell lines.

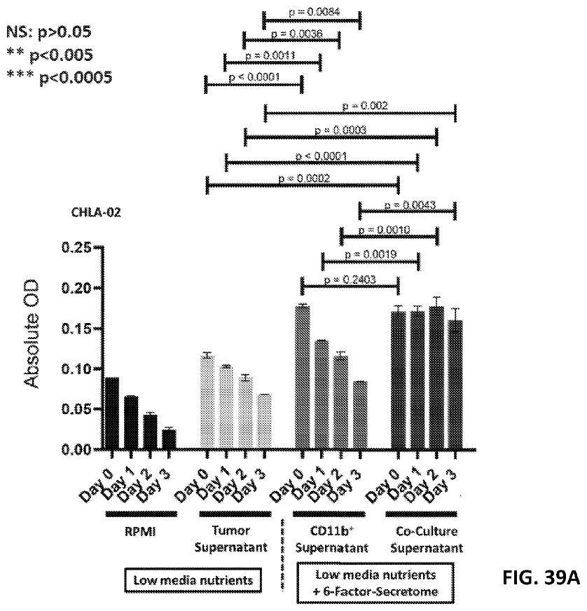

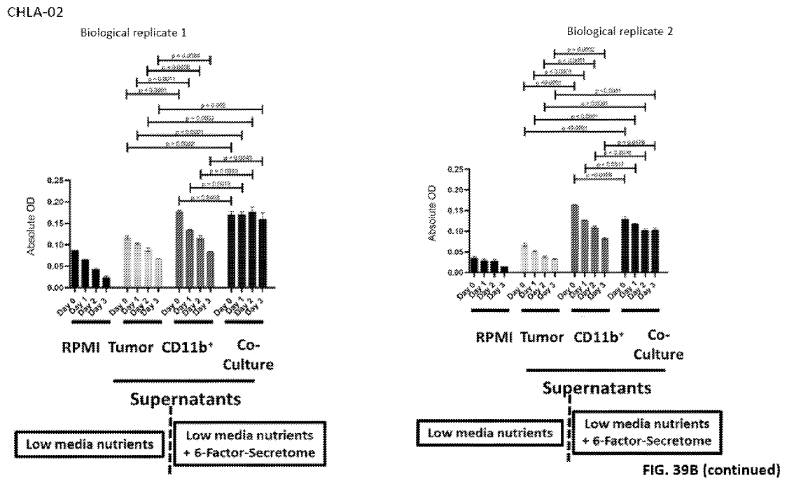

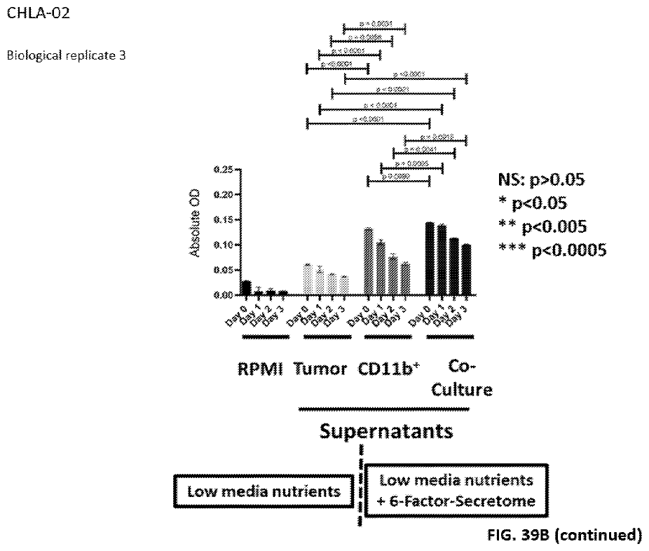

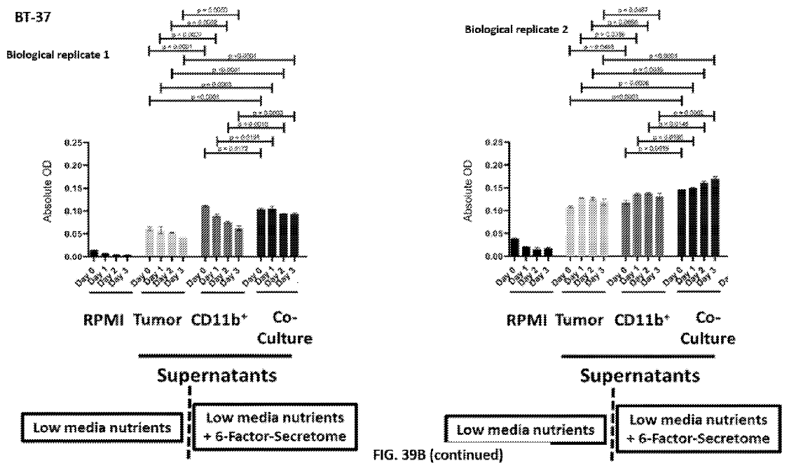

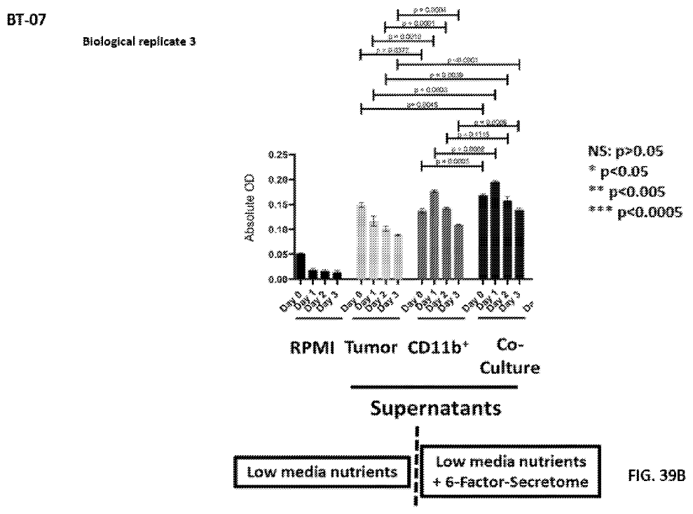

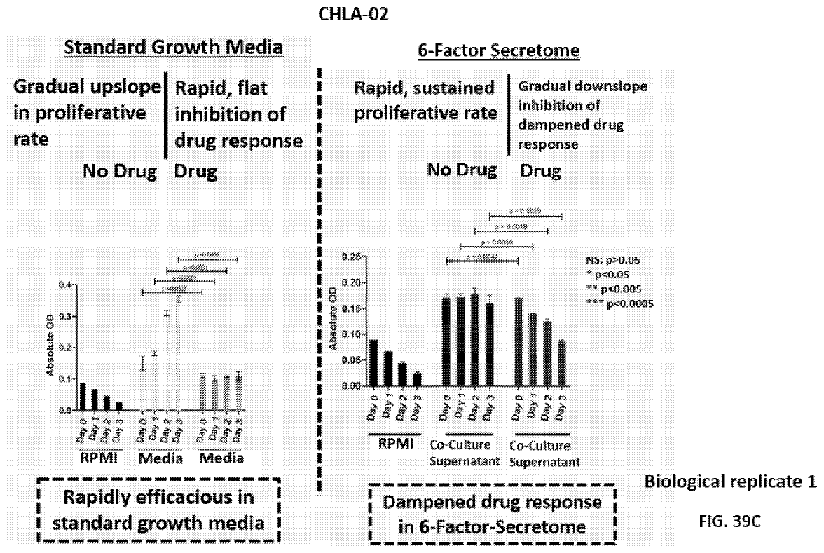

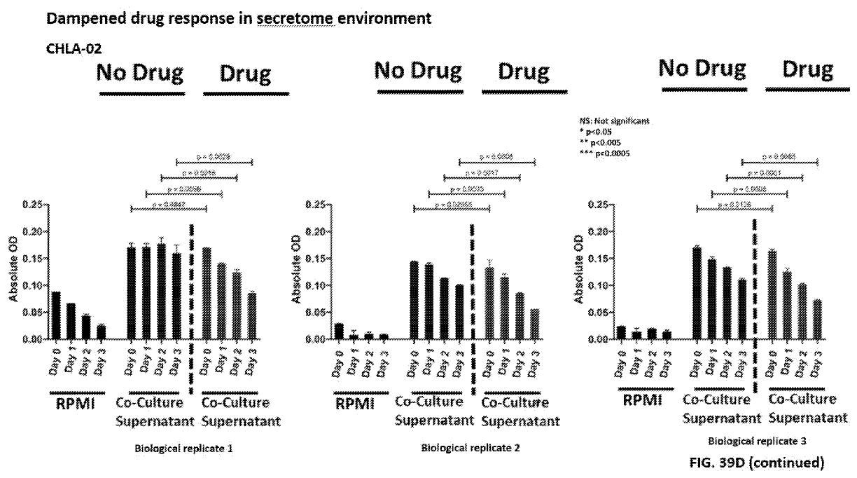

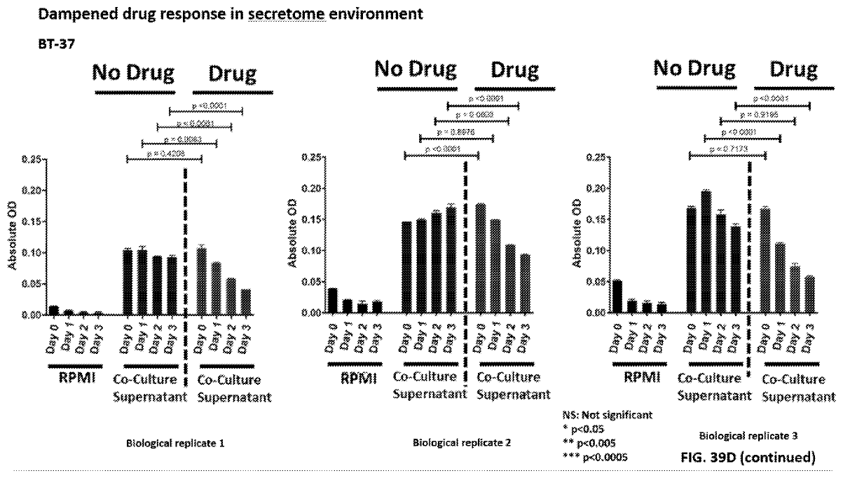

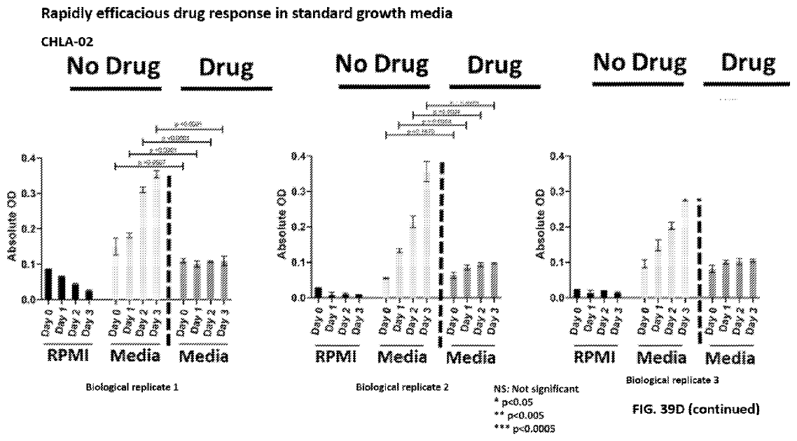

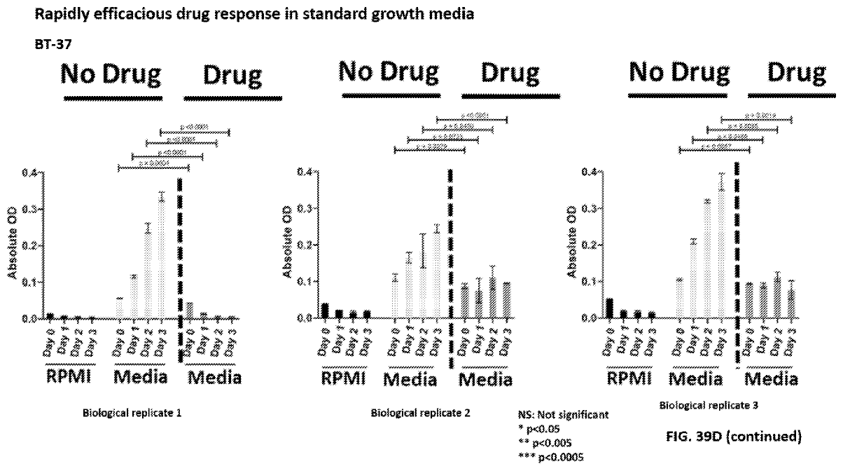

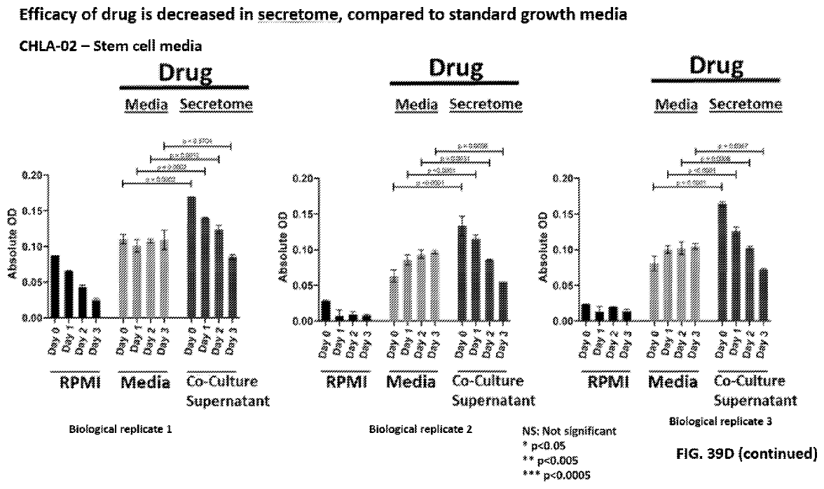

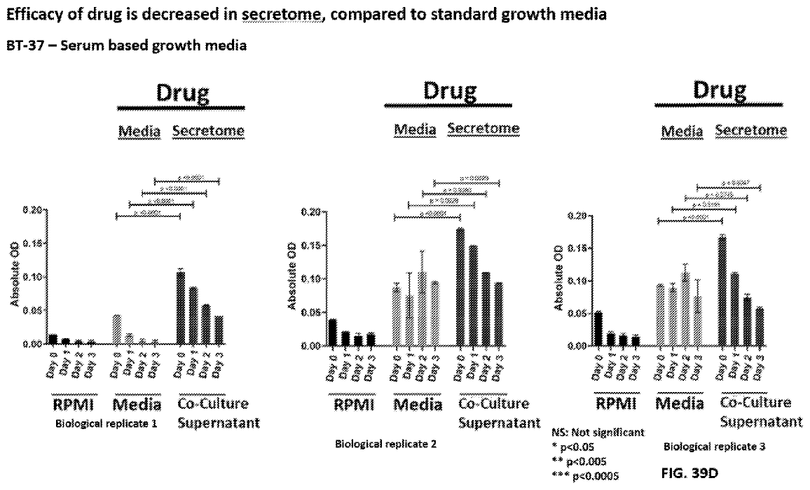

FIG. 4C and FIG. 4D show bar graphs that during resource-limited nutrient deprivation state, cancer cells can sustain their proliferation using this secretome. Secretome derived from CD11b+ macrophages co-cultured with tumour cells (CHLA-02 and BT-37) provided more sustained tumour proliferative effect compared to secretome derived from CD11b+ cells alone (actual p-values in FIG. 39).

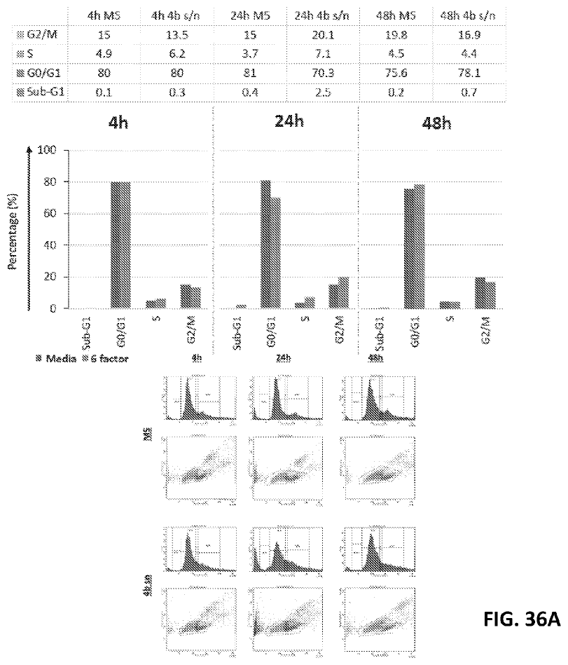

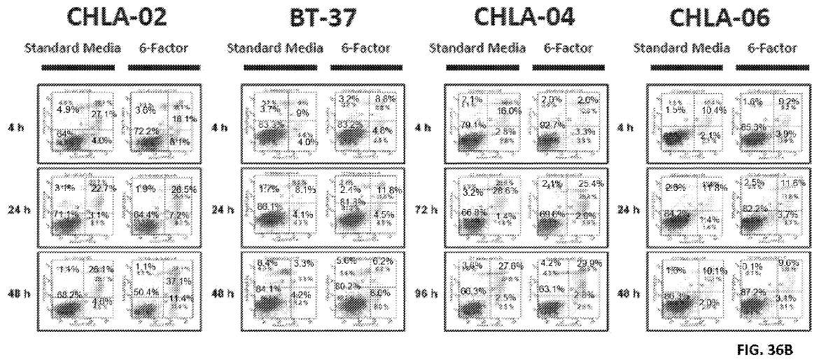

FIG. 4E shows flow cytometry using AnV/PI comparing cell death between standard growth media versus six-factor-secretome (FIG. 36).

FIG. 4F shows photomicrographs (Magnification 4X, 10X, 20X): Tumour cells rearrange to form extensive, interlacing, web-like structures connecting cells over long-distance radius (CHLA-02 in T75 flasks) in six-factor-secretome after 24 h of nutrient stress, in contrast to growth in clusters during nutrient-replete conditions (standard growth media).

FIG. 4G shows large image photomicrographs to capture large areas of tumour cells (CHLA-02) grown in T75 flask demonstrated the distinct interlacing web-like architecture of tumour cells aligning linearly in six-factor secretome (arrows, white irregular line; linear patterning) which contrasted with tumour cell clusters (white circles) growing in standard growth media (FIG. 13). Click enlarge “+” on FIG. 12B to view linear web-like patterning of tumour cells. Inset (box) zoomed in for enlarged views.

FIG. 4H shows a diagram depicting a quadruple mechanism of secretome capable of (1) inducing rapid early growth spurt and (2) preventing tumour cell death during nutrient stress, simultaneously (3) dampening drug response of tumour cells and maintaining stable interlacing web-like architecture of tumour cells when exposed to drug, provide strong advantages for tumour cell resilience and therapy resistance.

FIG. 5A shows a diagram and plots that maternal embryonic leucine kinase (MELK) is a universal target in 3 ATRT subtypes. Dot plots showing MELK expression in patient tumours, tumour cell lines, normal brain tissue control (fetal and childhood) in Discovery Set (RNA-seq, n=28) and Validation Set (Affymetrix U133 plus 2.0, n=49). Student T-Test. *: p<0.05,**: p<0.005. (FIG. 25). OTSSP167, a cell cycle inhibitor against MELK (in Phase 1/2 trials for adult cancers) was used to target tumour compartment common to patient tumours and tumour cell lines. Bar graphs showing qRT-PCR validation of MELK among ATRT cell lines (FIG. 25A).

FIG. 5B shows graphs with dose-response effect of OTSSP167 on six ATRT cell lines over 13 days. OTSSP167 was effective in nanomolar ranges (FIG. 26).

FIG. 5C and FIG. 5D show flow cytometry or western blot data of OTSSP167 induced apoptotic cell death of ATRT cells. Flow cytometry analysis for apoptotic cell death (Annexin V+PI−) and cell cycle analyses of OTSSP167-treated CHLA-02 (Day 4 IC50 dose—20.76 nM) and BT-37 (Day 4 IC50 dose-70.42 nM). Immunoblotting demonstrated p-Histone H2A.X was increased with 24 h, 48 h and 72h-OTSSP167 treatment in CHLA-02 and BT-37 cells, indicative of OTSSP167 inducing DNA-damage. c-PARP was also increased with OTSSP167 treatment, supporting activation of intrinsic apoptosis pathway.

FIG. 5E shows graphs and figures that in a drug-naïve state (DMSO-vehicle control), six-factor-secretome evoked a rapid growth spike, contrasted to standard growth media which promoted a gradual increase in tumour proliferation. In the presence of drug (OTSSP167), rapid growth inhibition was observed in standard growth media, contrasted to dampened drug response in secretome microenvironment. OTSSP167 efficacy was decreased in six-factor-secretome, compared to standard growth media. (*: p<0.005, **: p<0.0005, NS: Not significant). Actual p-values in FIG. 39.

FIG. 5F shows a graph with overall survival of animals that was significantly improved with OTSSP167 treatment in an aggressive autopsy-derived patient-derived orthotopic xenograft (PDOX) Model (p=0.032, Log-rank test).

FIG. 5G shows flow cytometry using AnV/PI comparing cell death of OTSSP167-treated BT-37 and CHLA in six-factor-secretome (FIG. 37, FIG. 38). OTSSP167-treated BT-37 cells undergo more cell death than CHLA-02 cells in secretome after 24-48 h.

FIG. 5H shows photomicrographs (Magnification 10×): Tumour cells treated with OTSSP167 in secretome maintained their interlacing, web-like architecture in contrast to tumour cells (CHLA-02) breaking up into smaller clusters when treated with OTSSP167 in standard growth media, suggesting a potential mechanism for therapy resistance provided by secretome.

FIG. 6 shows a graphical abstract with a schematic depicting implication of the study of the present disclosure on drug screening and patient relapse.

FIG. 7A shows a graph of patient tumours (Discovery Set) that were enriched in TREM2 and expression of TREM2 followed the trend of CD11b/ITGAM (pan-macrophage) and CD163macrophages (M2 macrophage), but not tumour cell lines. TREM2 expression was 12572-fold higher (patient tumours: cell lines. Student T Test. **: p<0.005) and CD206 expression 141-fold higher among patient tumours compared to cell lines (Student T-Test. **: p<0.005). TREM2 expression on control normal brain tissues was low, similar to CD11b/ITGAM and CD163.

FIG. 7B shows a graph of TREM2 expression across 3 ATRT-subtypes (Validation Set), median ranged between 6.8-7.6, with much less variation between subtypes compared to CD11b (median ranged 5-6.6, FIG. 3A) and CD163 (median ranged 7.6-10.2).

FIG. 7C shows a graph with CD206 expression on RNA-seq (Discovery Set) that was overall lower compared to TREM2 but followed a similar general trend to TREM2.

FIG. 7D shows a graph with CD206 expression across 3 ATRT-subtypes (Validation Set), with MYC-and TYR-ATRTs more enriched in CD206 compared to SHH-ATRTs. A similar trend was observed in CD11b/ITGAM and CD163 (FIG. 3B).

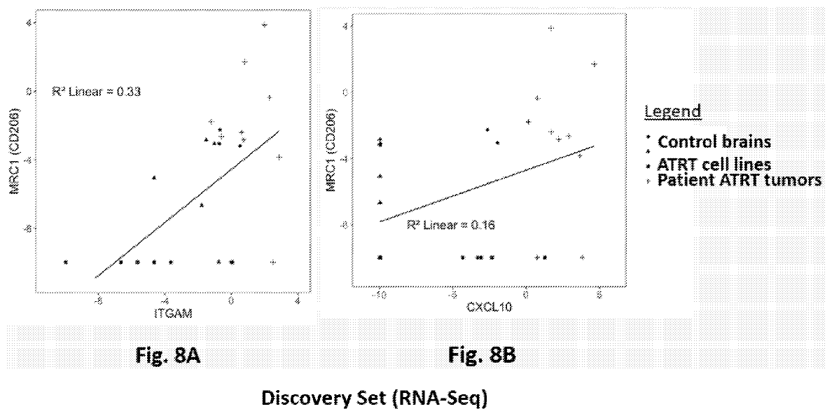

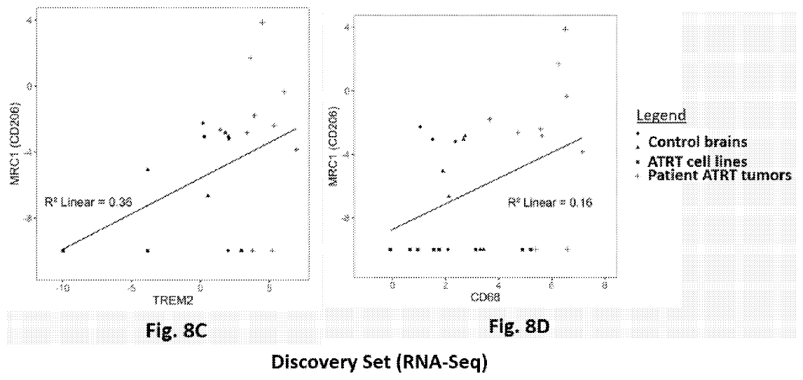

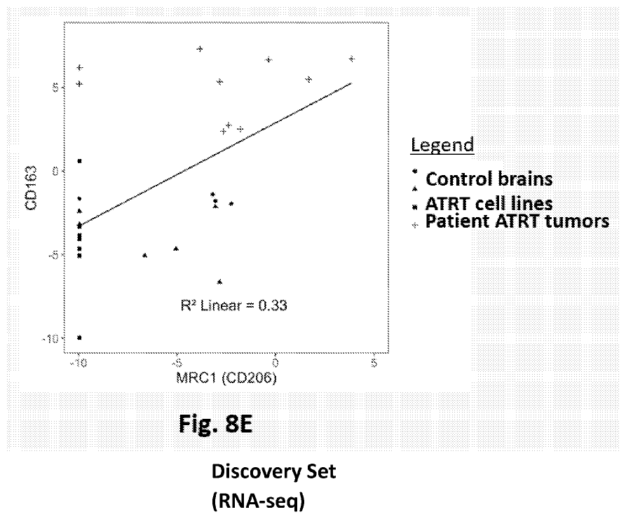

FIG. 8A shows correlation plots of CD206 against macrophage marker CD11b/ITGAM in patient tumours, patient-derived tumour cell lines of ATRT and normal brain controls, in Discovery Set (RNA-seq: patient tumours, tumour cell lines, normal brain tissues). Macrophage markers of various subsets correlated less well with CD206 on RNA-seq platform. Pearson correlation with p-value<0.05 and R-square<0.6 show that the two genes are highly positively correlated in gene expression.

FIG. 8B shows correlation plots of CD206 against macrophage marker CXCL10 in patient tumours, patient-derived tumour cell lines of ATRT and normal brain controls, in Discovery Set (RNA-seq: patient tumours, tumour cell lines, normal brain tissues). Macrophage markers of various subsets correlated less well with CD206 on RNA-seq platform. Pearson correlation with p-value<0.05 and R-square<0.6 show that the two genes are highly positively correlated in gene expression.

FIG. 8C shows correlation plots of CD206 against macrophage marker TREM2 in patient tumours, patient-derived tumour cell lines of ATRT and normal brain controls, in Discovery Set (RNA-seq: patient tumours, tumour cell lines, normal brain tissues). Macrophage markers of various subsets correlated less well with CD206 on RNA-seq platform. Pearson correlation with p-value<0.05 and R-square<0.6 show that the two genes are highly positively correlated in gene expression.

FIG. 8D shows correlation plots of CD206 against macrophage marker CD68 in patient tumours, patient-derived tumour cell lines of ATRT and normal brain controls, in Discovery Set (RNA-seq: patient tumours, tumour cell lines, normal brain tissues). Macrophage markers of various subsets correlated less well with CD206 on RNA-seq platform. Pearson correlation with p-value<0.05 and R-square<0.6 show that the two genes are highly positively correlated in gene expression.

FIG. 8E shows correlation plots of CD206 against macrophage marker CD163 in patient tumours, patient-derived tumour cell lines of ATRT and normal brain controls, in Discovery Set (RNA-seq: patient tumours, tumour cell lines, normal brain tissues). Macrophage markers of various subsets correlated less well with CD206 on RNA-seq platform. Pearson correlation with p-value<0.05 and R-square<0.6 show that the two genes are highly positively correlated in gene expression.

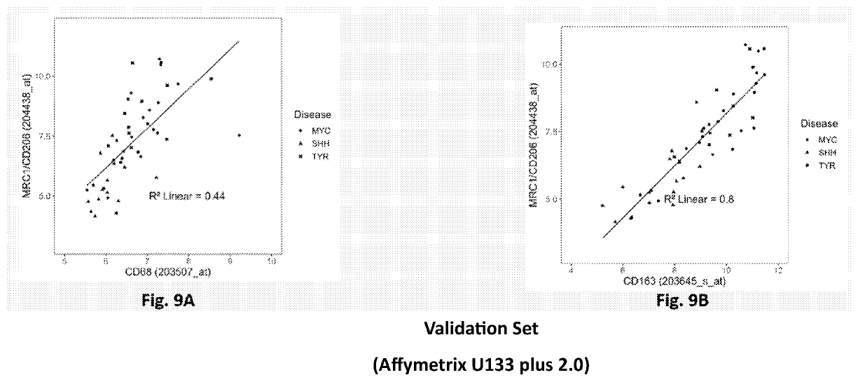

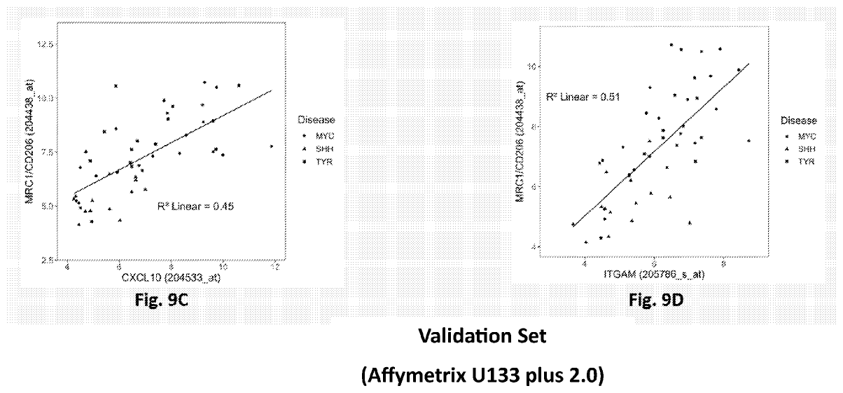

FIG. 9A shows correlation plots of CD206 against macrophage marker CD68 in patient tumours across 3 ATRT-subtypes, in Validation Set (Affymetrix U133 plus 2.0). CD206 correlated very well with CD163 (R-square=0.8) while other macrophage markers of various subsets (i.e., CD68, CXCL10, CD11b/ITGAM) correlated less well with CD206 on Affymetrix U133 plus 2.0 platform. Pearson correlation with p-value<0.05 and R-square<0.6 show that the two genes are highly positively correlated in gene expression.

FIG. 9B shows correlation plots of CD206 against macrophage marker CD163 in patient tumours across 3 ATRT-subtypes, in Validation Set (Affymetrix U133 plus 2.0). CD206 correlated very well with CD163 (R-square=0.8) while other macrophage markers of various subsets (i.e., CD68, CXCL10, CD11b/ITGAM) correlated less well with CD206 on Affymetrix U133 plus 2.0 platform. Pearson correlation with p-value<0.05 and R-square<0.6 show that the two genes are highly positively correlated in gene expression.

FIG. 9C shows correlation plots of CD206 against macrophage marker CXCL10 in patient tumours across 3 ATRT-subtypes, in Validation Set (Affymetrix U133 plus 2.0). CD206 correlated very well with CD163 (R-square=0.8) while other macrophage markers of various subsets (i.e., CD68, CXCL10, CD11b/ITGAM) correlated less well with CD206 on Affymetrix U133 plus 2.0 platform. Pearson correlation with p-value<0.05 and R-square<0.6 show that the two genes are highly positively correlated in gene expression.

FIG. 9D shows correlation plots of CD206 against macrophage marker CD11b/ITGAM, in patient tumours across 3 ATRT-subtypes, in Validation Set (Affymetrix U133 plus 2.0). CD206 correlated very well with CD163 (R-square=0.8) while other macrophage markers of various subsets (i.e., CD68, CXCL10, CD11b/ITGAM) correlated less well with CD206 on Affymetrix U133 plus 2.0 platform. Pearson correlation with p-value<0.05 and R-square<0.6 show that the two genes are highly positively correlated in gene expression.

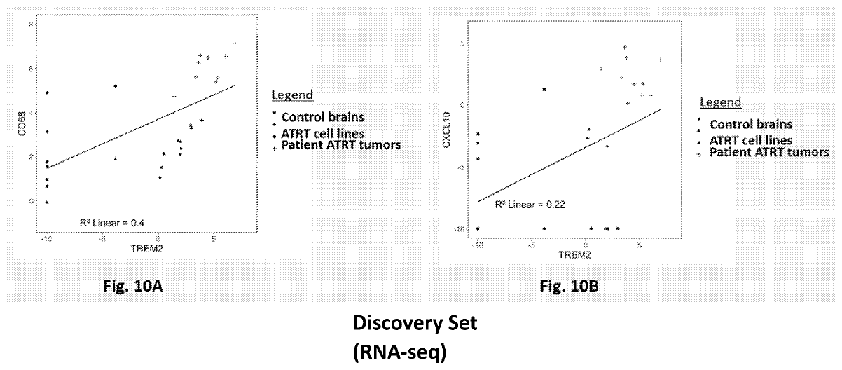

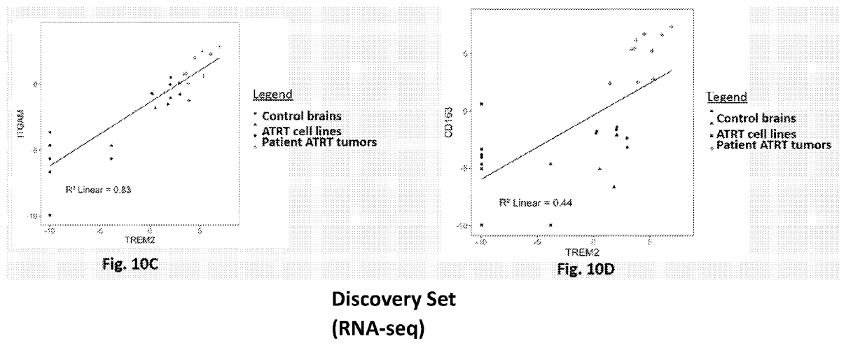

FIG. 10A shows correlation plots of TREM2 against macrophage marker CD68 in patient tumours, patient-derived tumour cell lines of ATRT and normal brain controls, in Discovery Set (RNA-seq: patient tumours, tumour cell lines, normal brain tissues). CD11b/IGTAM correlated very well with TREM2 (R-square=0.83) while other macrophage markers of various subsets (i.e., CD68, CXCL10, CD163) correlated less well with TREM2 on RNA-seq platform. Pearson correlation with p value<0.05 and R-square<0.6 show that the two genes are highly positively correlated in gene expression.

FIG. 10B shows correlation plots of TREM2 against macrophage marker CXCL10 in patient tumours, patient-derived tumour cell lines of ATRT and normal brain controls, in Discovery Set (RNA-seq: patient tumours, tumour cell lines, normal brain tissues). CD11b/ITGAM correlated very well with TREM2 (R-square=0.83) while other macrophage markers of various subsets (i.e., CD68, CXCL10, CD163) correlated less well with TREM2 on RNA-seq platform. Pearson correlation with p value<0.0.5 and R-square<0.6 show that the two genes are highly positively correlated in gene expression.

FIG. 10C shows correlation plots of TREM2 against macrophage marker CD11b/ITGAM in patient tumours, patient-derived tumour cell lines of ATRT and normal brain controls, in Discovery Set (RNA-seq: patient tumours, tumour cell lines, normal brain tissues). CD11b/IGTAM correlated very well with TREM2 (R-square=0.83) while other macrophage markers of various subsets (i.e., CD68, CXCL10, CD163) correlated less well with TREM2 on RNA-seq platform. Pearson correlation with p value<0.0.5 and R-square<0.6 show that the two genes are highly positively correlated in gene expression.

FIG. 10D shows correlation plots of TREM2 against macrophage marker CD163 in patient tumours, patient-derived tumour cell lines of ATRT and normal brain controls, in Discovery Set (RNA-seq: patient tumours, tumour cell lines, normal brain tissues). CD11b/ITGAM correlated very well with TREM2 (R-square=0.83) while other macrophage markers of various subsets (i.e., CD68, CXCL10, CD163) correlated less well with TREM2 on RNA-seq platform. Pearson correlation with p value<0.05 and R-square<0.6 show that the two genes are highly positively correlated in gene expression.

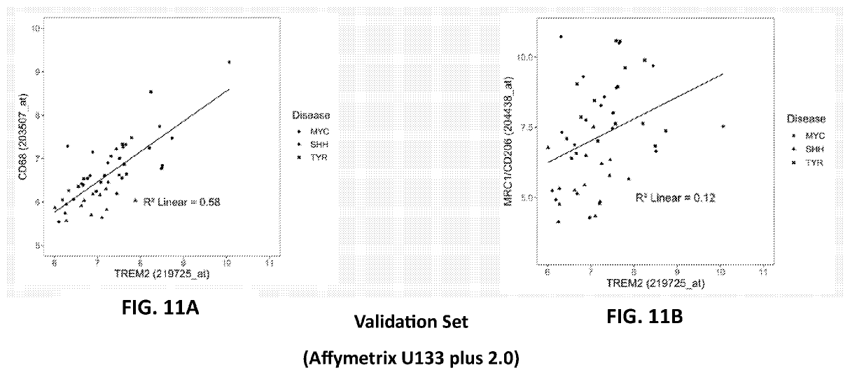

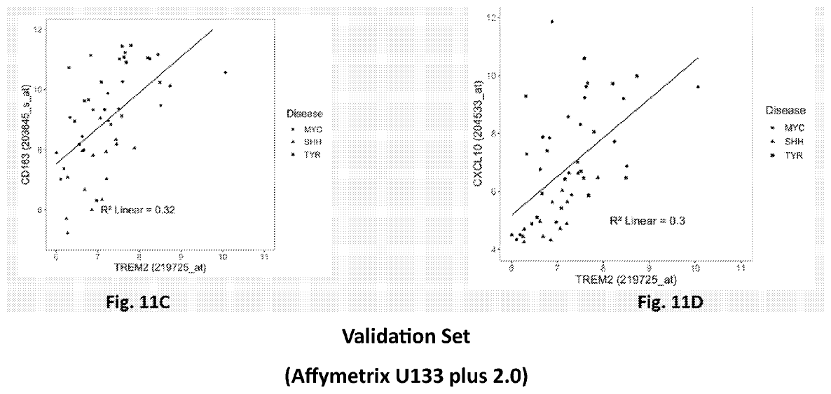

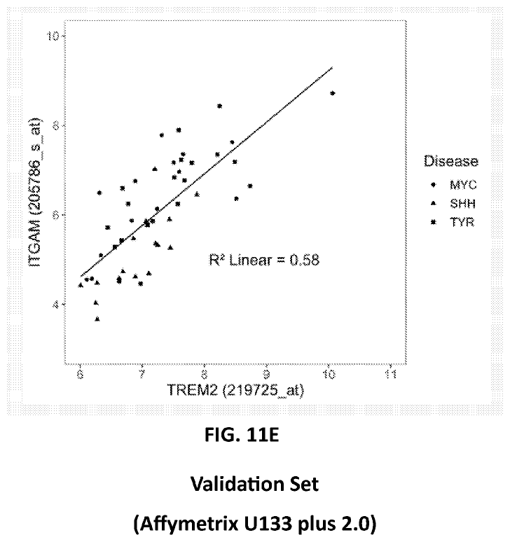

FIG. 11A shows correlation plots of TREM2 against macrophage marker CD68 in patient tumours across 3 ATRT-subtypes, in Validation Set (Affymetrix U133 plus 2.0). TREM2correlated fairly with CD11b/ITGAM (R-square=0.58), similar trend with Affymetrix U133 plus 2.0 (FIG. 10) but less well. TREM2 also correlated fairly with CD68 (R-square=0.57). Other macrophage markers of various subsets (i.e., CXCL10, CD163, CD206) correlated less well with TREM2 on Affymetrix U133 plus 2.0 platform. Pearson correlation with p-value<0.05 and R-square>0.6 show that the two genes are highly positively correlated in gene expression.

FIG. 11B shows correlation plots of TREM2 against macrophage marker CD206 in patient tumours across 3 ATRT-subtypes, in Validation Set (Affymetrix U133 plus 2.0). TREM2 correlated fairly with CD11b/ITGAM (R-square=0.58), similar trend with Affymetrix U133 plus 2.0 (FIG. 10) but less well. TREM2 also correlated fairly with CD68 (R-square=0.57). Other macrophage markers of various subsets (i.e., CXCL10, CD163, CD206) correlated less well with TREM2 on Affymetrix U133 plus 2.0 platform. Pearson correlation with p-value<0.05 and R-square<0.6 show that the two genes are highly positively correlated in gene expression.

FIG. 11C shows correlation plots of TREM2 against macrophage marker CD163 in patient tumours across 3 ATRT-subtypes, in Validation Set (Affymetrix U133 plus 2.0). TREM2 correlated fairly with CD11b/ITGAM (R-square=0.58), similar trend with Affymetrix U133 plus 2.0 (FIG. 10) but less well. TREM2 also correlated fairly with CD68 (R-square=0.57). Other macrophage markers of various subsets (i.e., CXCL10, CD163, CD206) correlated less well with TREM2 on Affymetrix U133 plus 2.0 platform. Pearson correlation with p-value<0.05 and R-square<0.6 show that the two genes are highly positively correlated in gene expression.

FIG. 11D shows correlation plots of TREM2 against macrophage marker CXCL10 in patient tumours across 3 ATRT-subtypes, in Validation Set (Affymetrix U133 plus 2.0). TREM2 correlated fairly with CD11b/ITGAM (R-square=0.58), similar trend with Affymetrix U133 plus 2.0 (FIG. 10) but less well. TREM2 also correlated fairly with CD68 (R-square=0.57). Other macrophage markers of various subsets (i.e., CXCL10, CD163, CD206) correlated less well with TREM2 on Affymetrix U133 plus 2.0 platform. Pearson correlation with p-value<0.0.5 and R-square<0.6 show that the two genes are highly positively correlated in gene expression.

FIG. 11E shows correlation plots of TREM2 against macrophage marker CD11b/ITGAM in patient tumours across 3 ATRT-subtypes, in Validation Set (Affymetrix U133 plus 2.0). TREM2 correlated fairly with CD11b/ITGAM (R-square=0.58), similar trend with Affymetrix U133 plus 2.0 (FIG. 10) but less well. TREM2 also correlated fairly with CD68 (R-square=0.57). Other macrophage markers of various subsets (i.e., CXCL10, CD163, CD206) correlated less well with TREM2 on Affymetrix U133 plus 2.0 platform. Pearson correlation with p-value<0.05 and R-square<0.6 show that the two genes are highly positively correlated in gene expression.

FIG. 12A and FIG. 12C show microscope pictures of CHLA-02 taken 24 h after incubation in standard growth media. Tumour cells grown in standard media formed clusters of various sizes (circle) whereas tumour cells grown in six-factor-secretome (FIGS. 12B and 12D) formed interlacing web-like stringy chains with cells aligning linearly (irregular white line). Click enlarge “+” on FIG. 12B to view linear web-like patterning of tumour cells. Inset (white box) zoomed in for enlarged views (2.6 mm×2.07 mm flask area). FIG. 12C show data that was acquired using a fluorescent microscope: 4 large photomicrographs were acquired per flask at each time point (24 h and 48 h). Each large photomicrograph covers 1.36 cm×1.08 cm flask area. Direction of photomicrographs captured was in the anticlockwise sequence shown in the diagram boxes below 1, 3, 4, 2. Each large photomicrograph is composed of 324(18×18) smaller photomicrographs stitched together by the microscope with 10% overlap. Each large photomicrograph=Total 324 smaller photomicrographs stitched together with 10% overlap.

FIG. 12B and FIG. 12D show microscope pictures of CHLA-02 that were taken 24 h after incubation in six-factor secretome. Tumour cells grown in standard media formed clusters of various sizes (circle) whereas tumour cells grown in six-factor-secretome formed interlacing web-like stringy chains with cells aligning linearly (irregular white line). Click enlarge “+” on

FIG. 12B to view linear web-like patterning of tumour cells. Inset (white box) zoomed in for enlarged views (2.6 mm×2.07 mm flask area). FIG. 12D show data that was acquired using a fluorescent microscope: 4 large photomicrographs were acquired per flask at each time point (24 h and 48 h). Each large photomicrograph covers 1.36 cm×1.08 cm flask area. Direction of photomicrographs captured was in the anticlockwise sequence shown in the diagram boxes below 1, 3, 4, 2. Each large photomicrograph is composed of 324 (18×18) smaller photomicrographs stitched together by the microscope with 10% overlap. Each large photomicrograph=Total 324 smaller photomicrographs stitched together with 10% overlap.

FIG. 12E show microscope pictures of CHLA-02 that were taken 48 h after incubation in standard growth media. Tumour cells grown in standard media formed clusters of various sizes (circle) whereas tumour cells grown in six-factor-secretome formed interlacing web-like stringy chains with cells aligning linearly (irregular white line). Click enlarge “+” on FIG. 12F to view linear web-like patterning of tumour cells. Inset (white box) zoomed in for enlarged views. Each large photomicrograph=Total 324 smaller photomicrographs stitched together with 10% overlap.

FIG. 12F shows microscope pictures of CHLA-02 that were taken 48 h after incubation in 6-factor secretome. Tumour cells grown in standard media formed clusters of various sizes (circle) whereas tumour cells grown in six-factor-secretome formed interlacing web-like stringy chains with cells aligning linearly (irregular white line). Click enlarge “+” on FIG. 12F to view linear web-like patterning of tumour cells. Inset (white box) zoomed in for enlarged views Each large photomicrograph=Total 324 smaller photomicrographs stitched together with 10% overlap.

FIG. 12G shows microscope pictures of CHLA-02 that were taken 48 h after incubation in standard growth media. Tumour cells grown in standard media formed clusters of various sizes (circle) whereas tumour cells grown in six-factor-secretome formed interlacing web-like stringy chains with cells aligning linearly (irregular white line). Click enlarge “+” on FIG. 12F to view linear web-like patterning of tumour cells.

FIG. 12H shows microscope pictures of CHLA-02 that were taken 48 h after incubation in six-factor-secretome. Tumour cells grown in standard media formed clusters of various sizes (circle) whereas tumour cells grown in six-factor-secretome formed interlacing web-like stringy chains with cells aligning linearly (irregular white line). Click enlarge “+” on FIG. 12F to view linear web-like patterning of tumour cells.

FIG. 13 shows an immunohistochemical survey on CD163+M2 macrophage infiltration within a mix bag of 13 paediatric brain tumours comprising of three histological brain tumour types, from a single institution. The inventor of the present disclosure found enrichment of CD163-expressing macrophages among a good proportion of patient tumours across different histological types. The inventor of the present disclosure observed that not all patient tumours within each histological type demonstrated high CD163 expression-two ATRT tumours in this cohort, one demonstrated CD163hi expression and the other CD163lo, supporting our findings on gene expression platforms in FIG. 3. This indicates that within a single histological type, patient tumours vary in macrophage infiltration.

FIG. 14A shows flow cytometry plots of the creation of CD11b+ human myeloid-derived cells. Unstimulated human myeloid-derived cells (U937) barely expressed CD11b. U937 cells are stimulated with different cytokines/chemokines that were suggested to influence macrophages in the presence of PMA. Different combinations of proteins were tested on U937 and PMA stimulation. IL-4 (arrow) induced the highest expression of CD11b (pan macrophage marker). Highest expression of CD11b (pan-macrophage marker) was achieved (80-90%) with PMA and IL-4 (10 ng/ml or 20n g/ml). (FIG. 2A). A variety of cytokines were tested with PMA to stimulate human myeloid-derived cells. Stimulation with PMA+IL-4 resulted in the highest population of CD11b+ cells.

FIG. 14B shows flow cytometry plots of CD163 (M2 macrophage marker) expression that was very low.

FIG. 15 shows microscopy images of the growth morphology of the large panel of 7 patient-tumour-derived cell lines of ATRT of the present disclosure and 1 patient-derived orthotopic xenograft (PDOX) cell line. Of 8 ATRT cell lines, CHLA-06 was the most aggressive and fastest growing in-vitro phenotype in the panel of ATRT tumour models of the present disclosure. ATRT95 was too slow growing and not suitable for in-vitro experiments. The inventor of the present disclosure selected CHLA-06, the most aggressive ATRT tumour cells from the panel of 7 ATRT cell line of the present disclosure to create the tumour compartment, based on the assumption that the most rapid growing in-vitro cell line will provide/secrete the most complete spectrum/concoction of tumour-promoting factors.

FIG. 16A shows graphs with expanded view of six-factor-secretome in Discovery Set (RNA-seq, n=28). Tumours expressed higher levels of six-factor-secretome. Probe for RANTES (CCL5) on RNA sequencing platform appeared to work less well on RNA-seq.

FIG. 16B shows graphs of Affymetrix U133 plus 2.0 platform in Validation Set, RANTES (CCL5) was expressed at higher levels in MYC-ATRTs and TYR-ATRTs, compared to SHH-ATRTs. Expanded view of six-factor-secretome in Validation Set (GSE70678; Affymetrix U133 plus 2.0, n=49) of ATRTs classified by molecular subtypes. MYC-ATRTs and TYR-ATRTs expressed higher levels of six-factor-secretome except for CXCL8 which is similarly expressed across 3 ATRT subtypes using Affymetrix U133 plus 2.0 platform.

FIG. 17A shows plots of correlation of pan-macrophage marker CD11b (ITGAM) with M2 macrophage markers (CD163, CD68) in Discovery Set (RNA-seq, n=28) and Validation Set (GSE 70678; Affymetrix U133 plus 2.0, n=49). CD11 b, CD163 and CD68 correlated well among tumour samples in Discovery Set, and in Validation Set (MYC-subtype, SHH-subtype and TYR-subtype) classified by molecular subtypes. Macrophage markers correlated poorly among cell lines and normal brain tissue controls (fetal and childhood brains) in Discovery Set.

FIG. 17B shows plots of pan-macrophage marker CD11b (ITGAM) correlated poorly with M1 macrophage marker (CXCL10) in both Discovery Set and Validation Set. Normal brain tissues and cell lines correlated poorly.

FIG. 17C shows plots of M2 macrophage markers (CD163 and CD68) correlated well with each other, among patient tumours in both Discovery Set and Validation Set.

FIG. 17D shows plots of_M1 macrophage marker (CXCL10) corelated well with both M2 macrophage markers (CD163 and CD68) patient tumours in Validation Set. Cell lines and normal brain tissues in Discovery Set, did not correlate well.

FIG. 17E shows tables of comparison of gene expression of macrophage markers in Discovery Set and Validation Set.

FIG. 18A shows graphs that among all 6 ATRT cell lines studied, only 2 cell lines (CHLA-02 and BT-37) demonstrated an obvious increase in optical density (OD) readings above the baseline OD readings of six-Factor-secretome. Supernatants derived from CD11b+ cell factors (FIG. 4A) and CD11b+ and tumour cell co-culture factors (FIG. 4B), resulted in higher tumour cell proliferation compared to tumour cell factors. Baseline absorbance (OD readings) of conditioned media from tumour cells (cell lines), CD11b+ cells or co-culture supernatants (CD11b+ cells+Tumour cells). Frozen (−80° C.) supernatants versus freshly processed supernatants did not differ in baseline OD (data not shown).

FIG. 18B shows microscopy images that conditioned media from cell lines, CD11b+ cells or co-culture supernatants (CD 11b+ cells+Tumour cells) were cell-free. Scale bar: 100 μm, magnification: 10×.

FIG. 19A shows a graph with minimal baseline optical density (OD) of six-factor-secretome. The inventor of the present disclosure compared the baseline absorbance (without tumour cells) of 20% (20 μl) RPMI: 80% (80 μl) six-factor secretome and 40% (40 μL) RPMI: 60% (60 μl) six-factor secretome, the difference was not statistically significant (p value>0.05, Welch's unpaired T-test). The inventor of the present disclosure employed the former ratio for the assays of the present disclosure. Absorbance (OD readings) in proliferations assays using six-factor secretome was therefore not attributable to baseline absorbance of the secretome.

FIG. 19B shows a graph with non-trypsinized macrophages and trypsinized macrophages. Macrophages were harvested with or without trypsinization, re-plated and secretome harvested. Six-factor secretome derived from non-trypsinized macrophages versus trypsinized macrophages at the same seeding density (p value<0.05, Welch's unpaired T test), induced CHLA-02 proliferation.

FIG. 19C shows a graph of six-factor-secretome harvested from increased macrophage seeding density did not lead to higher CHLA-02 proliferation rates. Macrophage seeding density was increased by 10% and 20% and treated with PMA+IL-4 for 48 hours. Macrophages were harvested in pure RPMI at the 110% density or 120% density. Increasing macrophage seeding density did not produce any significant increase in CHLA-02 proliferation.

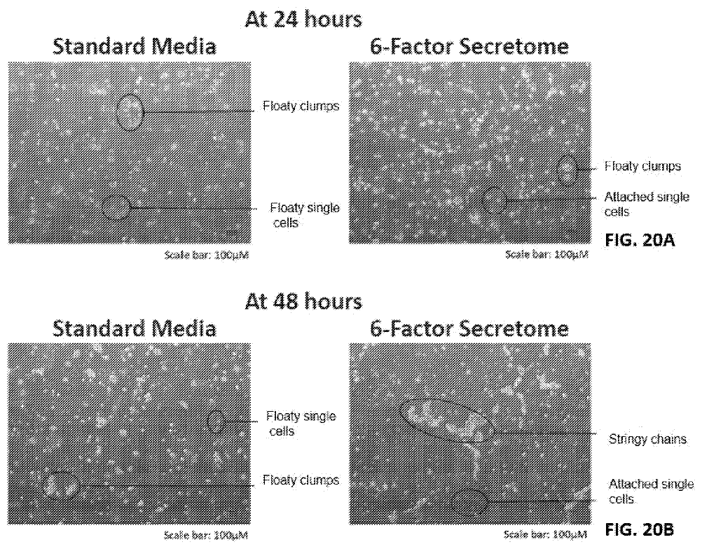

FIG. 20A shows microscopy images of chemo-attractant effects of six-factor-secretome on CHLA-02 cultured in T25 flasks. CHLA-02 cells grew in suspended clusters in standard growth media. CHLA-02 cells cultured in six-factor-secretome formed extensive, interlacing web-like structures connecting cells over long-distance radius (stringy chains) at 24 hours instead of cell clusters. Scale bar: 100 μm, magnification: 4×.

FIG. 20B shows microscopy images of chemo-attractant effects of 6-factor-secretome on CHLA-02 cultured in T25 flasks. CHLA-02 cells grew in suspended clusters in standard growth media. CHLA-02 cells cultured in six-factor-secretome formed extensive, interlacing web-like structures connecting cells over long-distance radius (stringy chains) at 48 hours, instead of cell clusters. Scale bar: 100 μm, magnification: 4×.

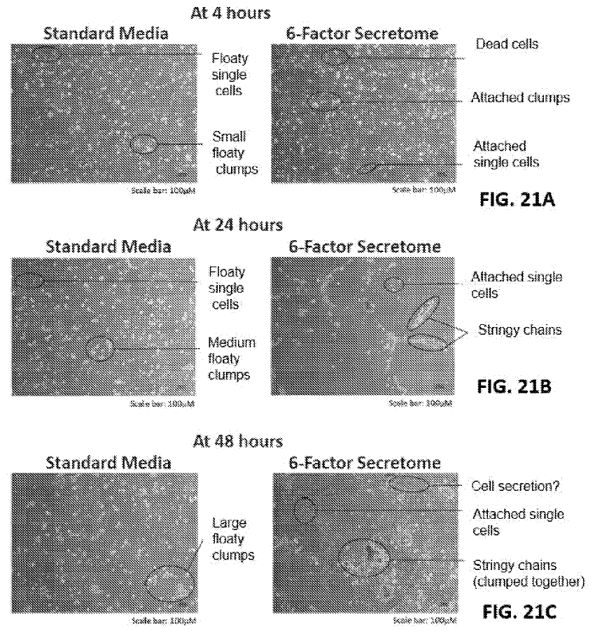

FIG. 21A shows microscopy images of chemo-attractant effects of six-factor-secretome on CHLA-02 cultured in T75 flasks. CHLA-02 cells grew in suspended clusters in standard growth media. CHLA- 02 cells cultured in six-factor-secretome formed cell clusters at 4 hours. Scale bar: 100 μm, magnification: 4×.

FIG. 21B shows microscopy images of chemo-attractant effects of six-factor-secretome on CHLA-02 cultured in T75 flasks. CHLA-02 cells grew in suspended clusters in standard growth media. CHLA-02 cells cultured in six-factor-secretome formed extensive, interlacing web-like structures connecting cells over long-distance radius (stringy chains) at 24 hours instead of cell clusters. Scale bar: 100 μm, magnification: 4×.

FIG. 21C shows microscopy images of chemo-attractant effects of six-factor-secretome on CHLA-02 cultured in T75 flasks. CHLA-02 cells grew in suspended clusters in standard growth media. CHLA-02 cells cultured in six-factor-secretome formed extensive, interlacing web-like structures connecting cells over long-distance radius (stringy chains) at 48 hours, instead of cell clusters. Scale bar: 100 μm, magnification: 4×.

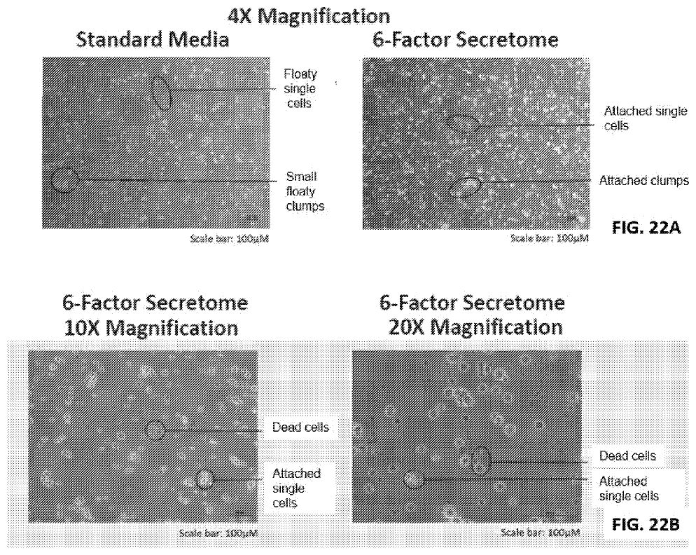

FIG. 22A shows microscopy images of the effects of 4-hour incubation of CHLA-02 in six-factor secretome cultured in T75 flasks observed under various magnification in T75 flasks. Replicate experiment: CHLA-02 cells grew in clusters in suspension, in standard culture media. CHLA-02 cells cultured in six-factor-secretome formed cell clusters at 4 hours under 4× magnification. Scale bar: 100 μm.

FIG. 22B shows microscopy images of the effects of 4-hour incubation of CHLA-02 in six-factor secretome cultured in T75 flasks observed under various magnification in T75 flasks. Replicate experiment: CHLA-02 cells grew in clusters in suspension, in standard culture media. CHLA-02 cells cultured in six-factor-secretome formed cell clusters at 4 hours under 10× and 20× magnification. Scale bar: 100 μm.

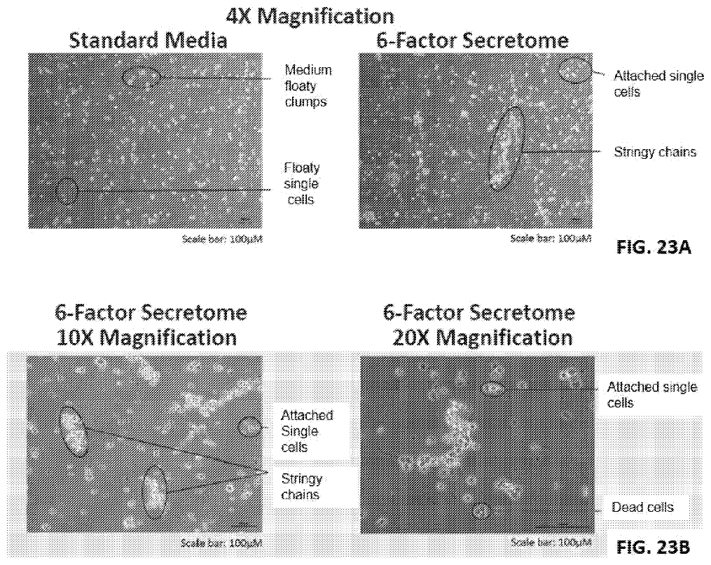

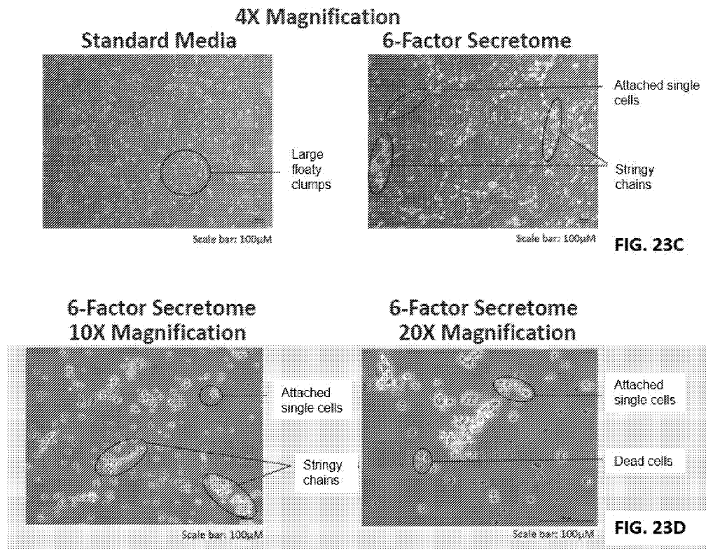

FIG. 23A shows microscopy images of the chemo-attractant effects of 24 hours incubation of CHLA-02 in six-factor secretome observed under various magnification in T75 flasks. Replicate experiment: CHLA-02 cells grew in clusters in suspension, in standard culture media. CHLA-02 cells cultured in six-factor-secretome formed extensive, interlacing web-like structures connecting cells over long-distance radius after 24 hours under 4× magnification. Scale bar: 100 μm.

FIG. 23B shows microscopy images of the chemo-attractant effects of 24 hours incubation of CHLA-02 in six-factor secretome observed under various magnification in T75 flasks. Replicate experiment: CHLA-02 cells grew in clusters in suspension, in standard culture media. CHLA-02 cells cultured in six-factor-secretome formed extensive, interlacing web-like structures connecting cells over long-distance radius after 24 hours under 10× and 20× magnification. Scale bar: 100 μm.

FIG. 23C shows microscopy images of the chemo-attractant effects of 48 hours incubation of CHLA-02 in six-factor-secretome observed under various magnification in T75 flasks. Replicate experiment: CHLA-02 cells grew in clusters in suspension, in standard culture media. CHLA-02 cells cultured in six-factor-secretome formed extensive, interlacing web-like structures connecting cells over long-distance radius after 48 hours under 4× magnification. Scale bar: 100 μm.

FIG. 23D shows microscopy images of the chemo-attractant effects of 48 hours incubation of CHLA-02 in six-factor-secretome observed under various magnification in T75 flasks. Replicate experiment: CHLA-02 cells grew in clusters in suspension, in standard culture media. CHLA-02 cells cultured in six-factor-secretome formed extensive, interlacing web-like structures connecting cells over long-distance radius after 48 hours under 10× and 20× magnification. Scale bar: 100 μm.

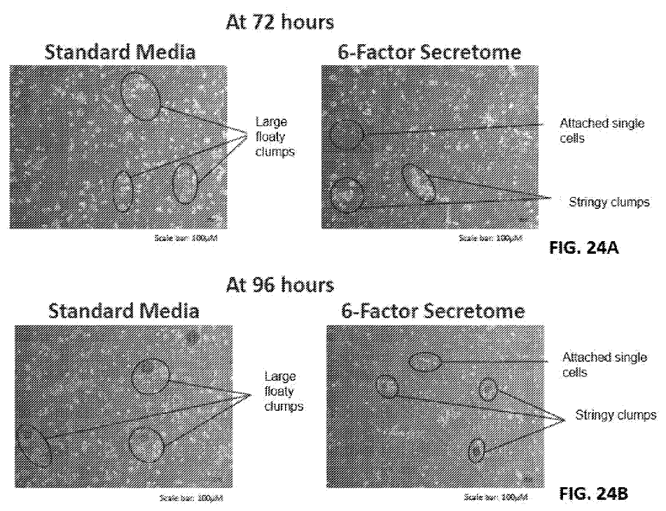

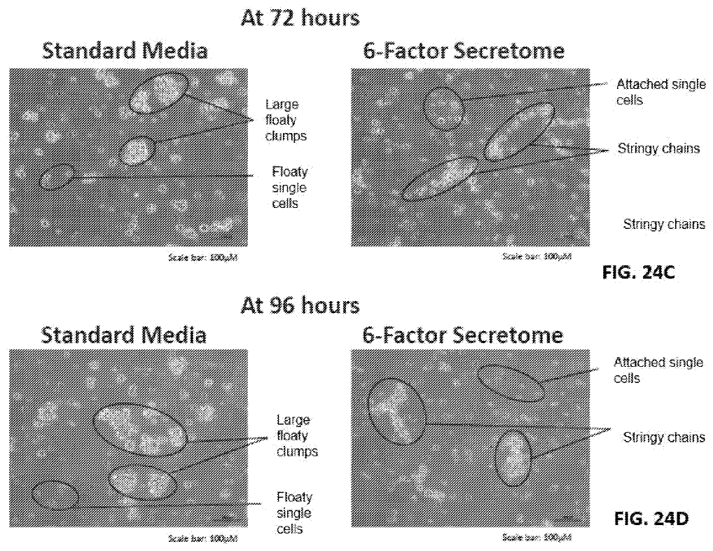

FIG. 24A shows microscopy images of the effects of six-factor-secretome on CHLA-02 cultured in T75 flasks at longer timepoints. CHLA-02 cultured in 6-factor-secretome formed cell clusters at longer timepoints as stringy chains of CHLA-02 cells became less prominent (disintegrated) at 72 hours. Scale bar: 100 μm, magnification: 4×.

FIG. 24B shows microscopy images of the effects of six-factor-secretome on CHLA-02 cultured in T75 flasks at longer timepoints. CHLA-02 cultured in six-factor-secretome formed cell clusters at longer timepoints as stringy chains of CHLA-02 cells became less prominent (disintegrated) at 96 hours. Scale bar: 100 μm, magnification: 4×.

FIG. 24C shows microscopy images of the effects of six-factor-secretome at longer timepoints cultured in T75 flasks at higher magnification. CHLA-02 cultured in six-factor secretome formed cell clusters at longer timepoints as stringy chains of CHLA-02 cells became less prominent (disintegrated) at 72 hours. Scale bar: 100 μm, magnification: 10×.

FIG. 24D shows microscopy images of the effects of six-factor-secretome at longer timepoints cultured in T75 flasks at higher magnification. CHLA-02 cultured in six-factor secretome formed cell clusters at longer timepoints as stringy chains of CHLA-02 cells became less prominent (disintegrated) at 96 hours. Scale bar: 100 μm, magnification: 10×.

FIG. 25A shows bar graphs showing qRT-PCR validation of MELK among ATRT cell lines and cell lines from other embryonal brain tumour types.

FIG. 25B shows a table with the comparison of MELK expression in Discovery Set and Validation Set. p-values were calculated using Student T-Test.

FIG. 26 shows a line graph of OTSSP167 that was effective against MELK-high BT-37 cells (5 nM dose) and MELK-low CHLA-04 (50 nM dose).

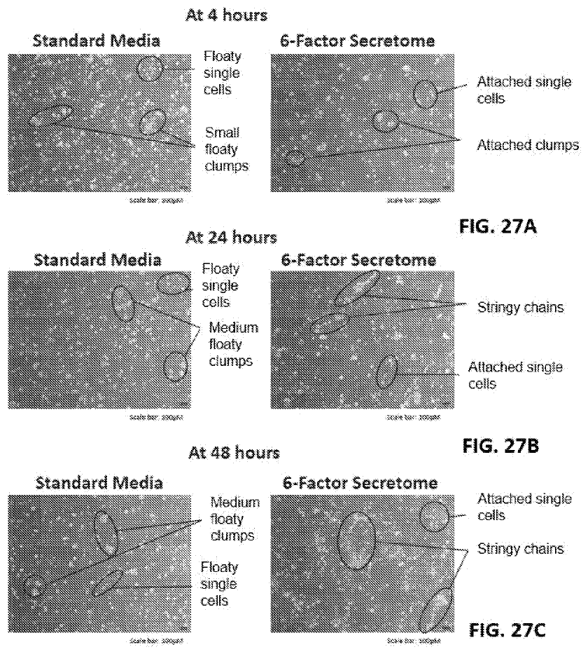

FIG. 27A shows microscopy images of the effects of OTSSP167 on CHLA-02 in six-factor-secretome cultured in T75 flasks. OTSSP167 treated CHLA-02 cells grew in clusters in suspension, in standard growth media. Treatment of CHLA-02 cells with OTSSP167 in six-factor-secretome formed cell clusters at 4 hours. Scale bar: 100 μm, magnification 4×.

FIG. 27B shows microscopy images of the effects of OTSSP167 on CHLA-02 in six-factor-secretome cultured in T75 flasks. OTSSP167 treated CHLA-02 cells grew in clusters in suspension, in standard growth media. Treatment of CHLA-02 cells with OTSSP167 in six-factor-secretome formed stringy chains at 24 hours. Scale bar: 100 μm, magnification 4×.

FIG. 27C shows microscopy images of the effects of OTSSP167 on CHLA-02 in six-factor-secretome cultured in T75 flasks. OTSSP167 treated CHLA-02 cells grew in clusters in suspension, in standard growth media. Treatment of CHLA-02 cells with OTSSP167 in six-factor-secretome formed even more prominent stringy chains which were afloat at 48 hours. Scale bar: 100 μm, magnification 4×.

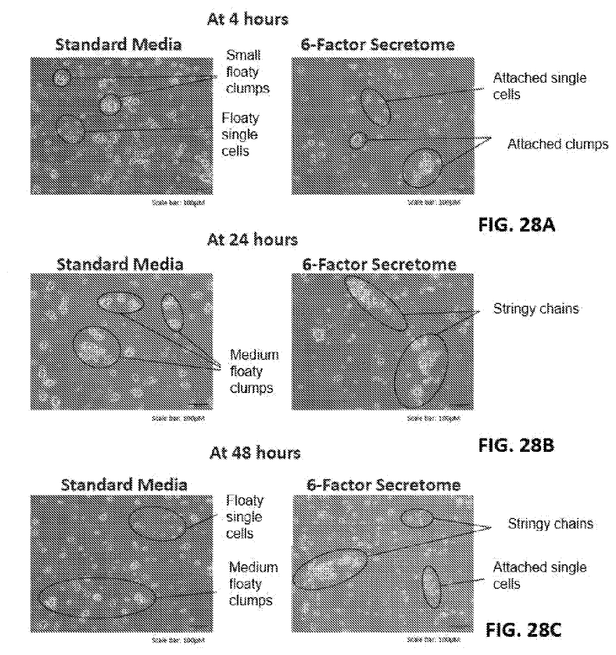

FIG. 28A shows microscopy images of the effects of OTSSP167 on CHLA-02 in six-factor-secretome cultured in T75 flasks at higher magnification. OTSSP167 treated CHLA-02 cells grow in clusters in suspension, in standard culture media. Treatment of CHLA-02 cells with OTSSP167 in six-factor secretome showed cell clusters at 4 hours. Scale bar: 100 μm, magnification: 10×.

FIG. 28B shows microscopy images of the effects of OTSSP167 on CHLA-02 in six-factor-secretome cultured in T75 flasks at higher magnification. OTSSP167 treated CHLA-02 cells grow in clusters in suspension, in standard culture media. Treatment of CHLA-02 cells with OTSSP167 in six-factor secretome formed stringy chains at 24 hours. Scale bar: 100 μm, magnification: 10×.

FIG. 28C shows microscopy images of the effects of OTSSP167 on CHLA-02 in six-factor-secretome cultured in T75 flasks at higher magnification. OTSSP167 treated CHLA-02 cells grow in clusters in suspension, in standard culture media. Treatment of CHLA-02 cells with OTSSP167 in six-factor secretome s formed even more prominent stringy chains afloat at 48 hours. Scale bar: 100 μm, magnification: 10×.

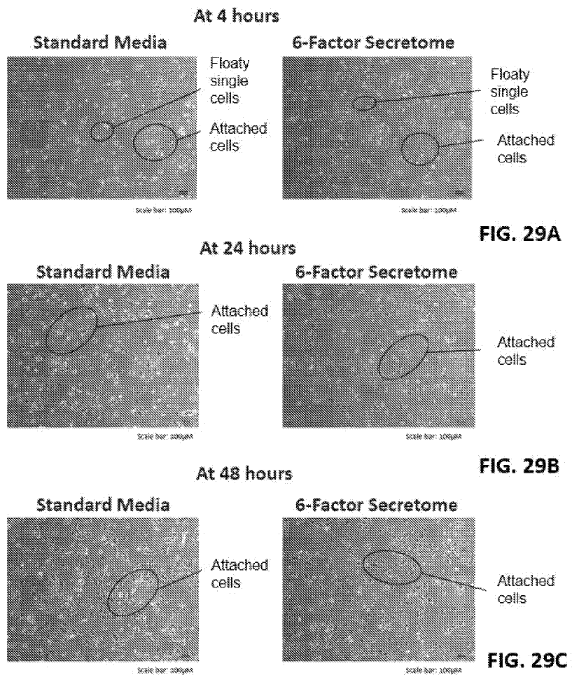

FIG. 29A shows microscopy images with no difference in gross morphology of BT-37 cells cultured in standard culture media versus six-factor secretome in T75 flasks. BT-37 cells exhibited mixed morphology-grew in clusters, monolayer and suspended clusters in standard growth media. BT-37 cells cultured in six-factor secretome showed no obvious difference compared to standard growth media at 4 hours. Scale bar: 100 μm, magnification: 4×.

FIG. 29B shows microscopy images with no difference in gross morphology of BT-37 cells cultured in standard culture media versus six-factor secretome in T75 flasks. BT-37 cells exhibited mixed morphology-grew in clusters, monolayer and suspended clusters in standard growth media. BT-37 cells cultured in six-factor secretome showed no obvious difference compared to standard growth media at 24 hours. Scale bar: 100 μm, magnification: 4×.

FIG. 29C shows microscopy images with no difference in gross morphology of BT-37 cells cultured in standard culture media versus six-factor secretome in T75 flasks. BT-37 cells exhibited mixed morphology-grew in clusters, monolayer and suspended clusters in standard growth media. BT-37 cells cultured in six-factor secretome showed no obvious difference compared to standard growth media at 48 hours. Scale bar: 100 μm, magnification: 4×.

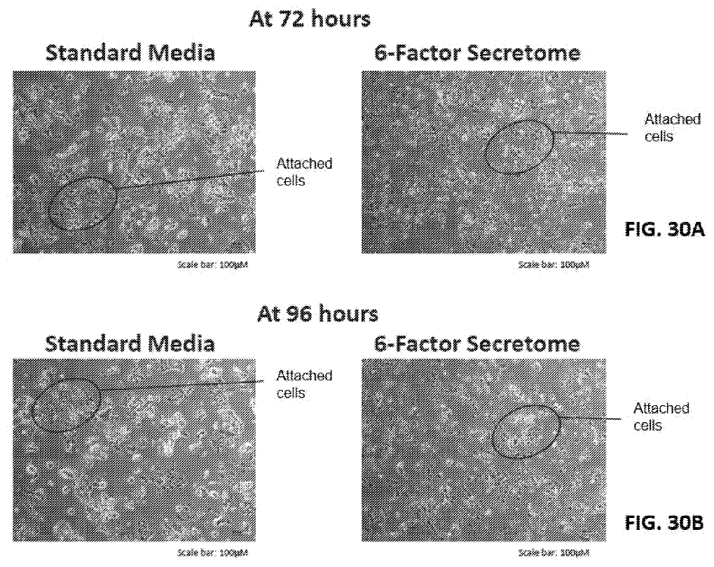

FIG. 30A shows microscopy images with no difference in gross morphology of BT-37 cultured in standard culture media versus six-factor-secretome in T75 flasks at longer timepoints. BT-37 cells exhibited mixed morphology-grew in clusters, monolayer and suspended clusters, in standard growth media. BT-37 cells cultured in six-factor-secretome appeared more sparse, but otherwise no obvious differences compared to standard growth media at 72 hours. Scale bar: 100 μm, magnification: 4×.

FIG. 30B shows microscopy images with no difference in gross morphology of BT-37 cultured in standard culture media versus six-factor-secretome in T75 flasks at longer timepoints. BT-37 cells exhibited mixed morphology-grew in clusters, monolayer and suspended clusters, in standard growth media. BT-37 cells cultured in six-factor-secretome appeared more sparse, but otherwise no obvious differences compared to standard growth media at 96 hours. Scale bar: 100 μm, magnification: 4×.

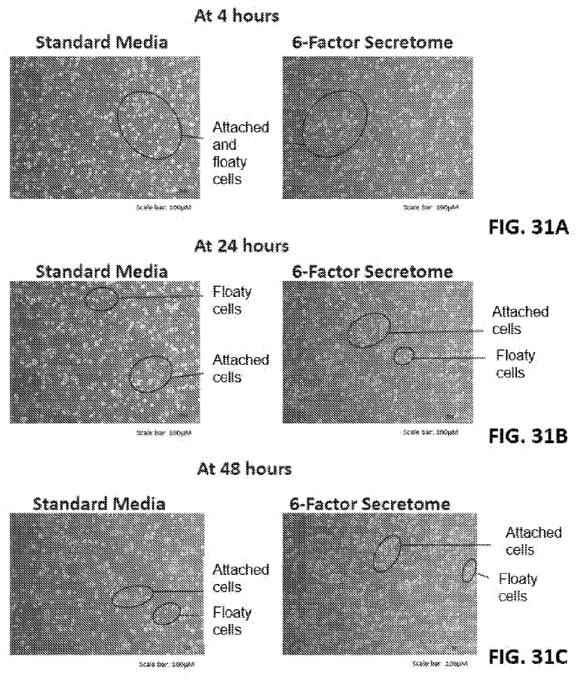

FIG. 31A shows microscopy images with more clumps of OTSSP167-treated BT-37 in T75 flasks observed in six-factor secretome at longer timepoints. OTSSP167-treated BT-37 cells exhibit mixed morphology—grow in cluster in monolayer and in suspension, in standard culture media. Treatment of BT-37 cells with OTSSP167 in six-factor secretome had no obvious difference to standard culture media at 4 hours. Scale bar: 100 μm, magnification: 4×.FIG. 31B shows microscopy images with more clumps of OTSSP167-treated BT-37 in T75 flasks observed in six-factor secretome at longer timepoints. OTSSP167-treated BT-37 cells exhibit mixed morphology—grow in cluster in monolayer and in suspension, in standard culture media. Treatment of BT-37 cells with OTSSP167 in six-factor secretome formed more prominent clumps at 24 hours. Scale bar: 100 μm, magnification: 4×.

FIG. 31C shows microscopy images with more clumps of OTSSP167-treated BT-37 in T75 flasks observed in six-factor secretome at longer timepoints. OTSSP167-treated BT-37 cells exhibit mixed morphology—grow in cluster in monolayer and in suspension, in standard culture media. Treatment of BT-37 cells with OTSSP167 in six-factor secretome formed more prominent clumps at 48 hours. Scale bar: 100 μm, magnification: 4×.

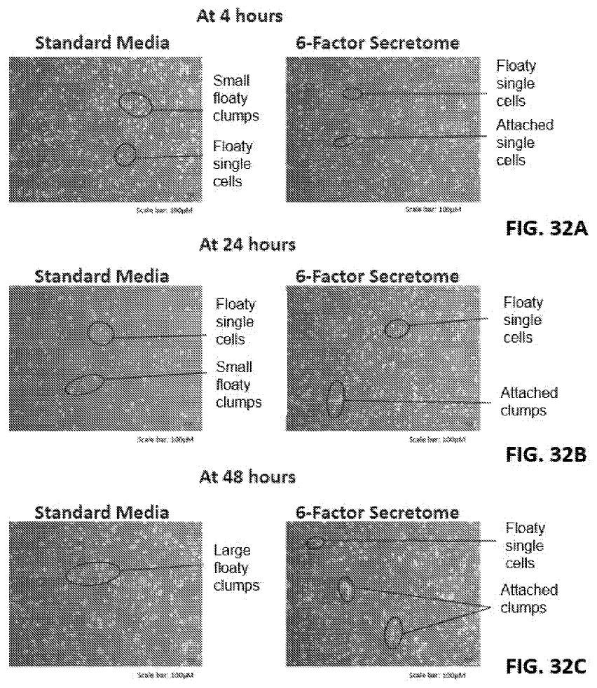

FIG. 32A shows microscopy images with CHLA-04 formed adherent cells in six-factor-secretome in T75 flasks. CHLA-04 cells grew in suspended clusters, in standard growth media. CHLA-04 cells cultured in six-factor-secretome exhibited growth morphology of adherent cells and floaty single cells at 4 hours. Scale bar: 100 μm, magnification: 4×.

FIG. 32B shows microscopy images with CHLA-04 formed adherent cells in six-factor-secretome in T75 flasks. CHLA-04 cells grew in suspended clusters, in standard growth media. CHLA-04 cells cultured in six-factor-secretome formed adherent clumps at 24 hours. Scale bar: 100 μm, magnification: 4×.

FIG. 32C shows microscopy images with CHLA-04 formed adherent cells in six-factor-secretome in T75 flasks. CHLA-04 cells grew in suspended clusters, in standard growth media. CHLA-04 cells cultured in six-factor-secretome formed adherent clumps at 48 hours. Scale bar: 100 μm, magnification: 4×.

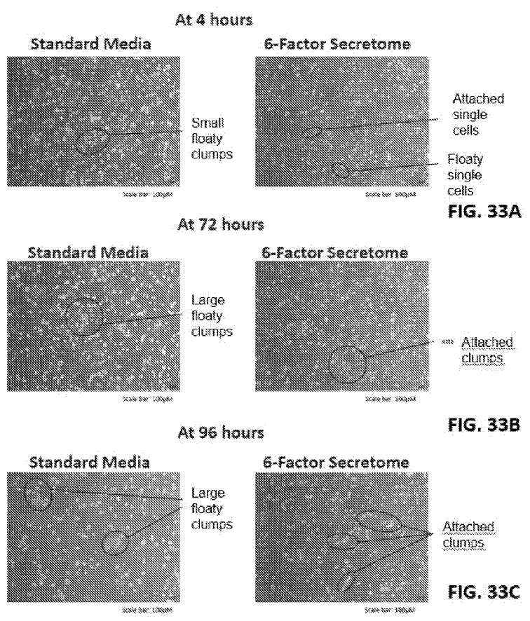

FIG. 33A shows microscopy images with CHLA-04 forming smaller cell clumps in six-factor-secretome in T75 flasks at longer timepoints. CHLA-04 grew in small floaty clumps in standard growth media at 4 hours. Scale bar: 100 μm, magnification: 4×.

FIG. 33B shows microscopy images with CHLA-04 forming smaller cell clumps in six-factor-secretome in T75 flasks at longer timepoints. CHLA-04 cells cultured in six-factor-secretome formed smaller adherent clusters at longer timepoints, as compared to cell clusters in standard growth media condition at 72 hours. Scale bar: 100 μm, magnification: 4×.

FIG. 33C shows microscopy images with CHLA-04 forming smaller cell clumps in six-factor-secretome in T75 flasks at longer timepoints. CHLA-04 cells cultured in six-factor-secretome formed smaller adherent clusters at longer timepoints, as compared to cell clusters in standard growth media condition at 96 hours. Scale bar: 100 μm, magnification: 4×.

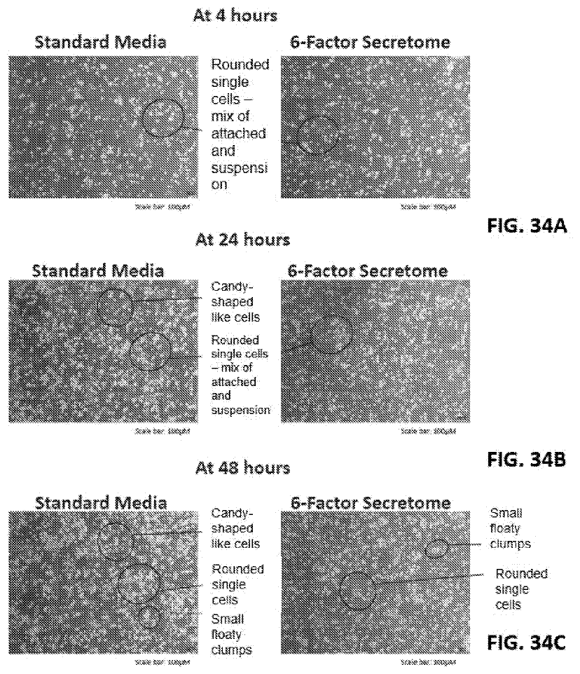

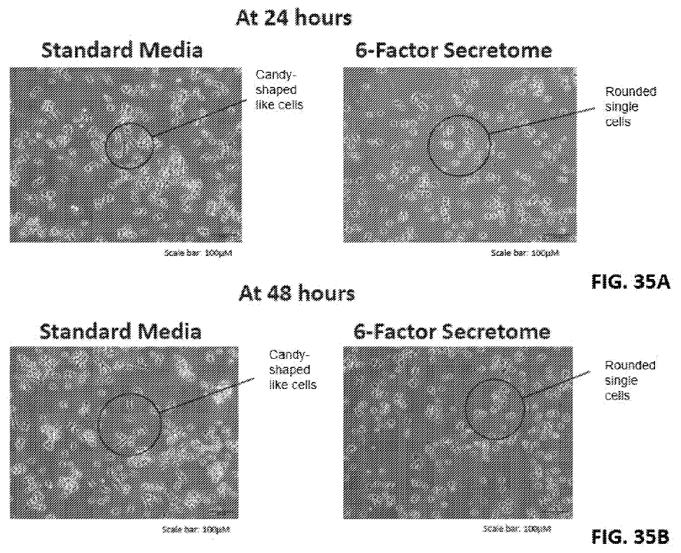

FIG. 34A shows microscopy images with low magnification demonstrating a similar growth morphology of CHLA-06 in standard growth media and in six-factor-secretome in T75 flasks. CHLA-06 cells exhibited mixed morphology in standard growth media—cells grew in single cell morphology, either attached or in suspension. CHLA-6 cells were less confluent when cultured in six-factor-secretome, but otherwise showed no difference in morphology as compared to standard growth media at 4 hours, (b) 24 hours and (c) 48 hours. Scale bar: 100 μm, magnification: 4×.

FIG. 34B shows microscopy images with low magnification demonstrating a similar growth morphology of CHLA-06 in standard growth media and in six-factor-secretome in T75 flasks. CHLA-06 cells exhibited mixed morphology in standard growth media—cells grew in single cell morphology, either attached or in suspension. CHLA-6 cells were less confluent when cultured in six-factor-secretome, but otherwise showed no difference in morphology as compared to standard growth media at 24 hours. Scale bar: 100 μm, magnification: 4×.

FIG. 34C shows microscopy images with low magnification demonstrating a similar growth morphology of CHLA-06 in standard growth media and in six-factor-secretome in T75 flasks. CHLA-06 cells exhibited mixed morphology in standard growth media—cells grew in single cell morphology, either attached or in suspension. CHLA-6 cells were less confluent when cultured in six-factor-secretome, but otherwise showed no difference in morphology as compared to standard growth media at 48 hours. Scale bar: 100 μm, magnification: 4×.

FIG. 35A shows microscopy images with high magnification demonstrating changes in morphology of CHLA-06 in six-factor secretome in T75 flasks. CHLA-06 cells were attached and in suspension and exhibited candy-like morphology when cultured in standard growth media, but when cultured in six-factor-secretome, formed rounded single cells in suspension and were less confluent, at 24 hours. Scale bar: 100 μm, magnification: 10×.

FIG. 35B shows microscopy images with high magnification demonstrating changes in morphology of CHLA-06 in six-factor secretome in T75 flasks. CHLA-06 cells were attached and in suspension and exhibited candy-like morphology when cultured in standard growth media, but when cultured in six-factor-secretome, formed rounded single cells in suspension and were less confluent, at 48 hours. Scale bar: 100 μm, magnification: 10×.

FIG. 36A shows a bar graph with unremarkable cell cycle changes in BT-37 when compared in standard growth media or in six-factor-secretome, by flow cytometry using PI.

FIG. 36B shows flow cytometry plots using AnV/PI comparing cell death between standard growth media versus six-factor-secretome (FIG. 4E).

FIG. 37A shows a bar chart and flow cytometry plots where in treatment-naïve state, cell death (apoptosis and necrosis) follows a similar trend in secretome and standard growth media conditions, from 4-48 h, with greater cell death at longer timepoint 48 h.

FIG. 37B shows a bar chart and flow cytometry plots where with prolongation over 72-96 h, cell death is more marked in secretome compared to standard growth media conditions.

FIG. 37C shows a bar chart and flow cytometry plots where with OTSSP167 treatment, cell death is more marked in secretome compared to standard growth media conditions, and the percentage of cell death is greater at longer time-points of 24-48 h.

FIG. 38A shows a bar chart and flow cytometry plots where in treatment-naïve state, cell death (apoptosis and necrosis) follows a similar trend in secretome and standard growth media conditions, from 4-48 h, with greater cell death at longer timepoint 48 h.

FIG. 38B shows a bar chart and flow cytometry plots with prolongation over 72-96 h, cell death is more marked in secretome compared to standard growth media conditions.

FIG. 38C shows a bar chart and flow cytometry plots with OTSSP167 treatment, cell death is more marked in secretome compared to standard growth media conditions, and the percentage of cell death is greater at longer time-points of 24-48 h.

FIG. 39A shows a bar graph with p-values of the graph shown in FIG. 4C.

FIG. 39B shows bar graphs with p-values of graphs shown in FIG. 4D.

FIG. 39C shows bar graphs with p-values of graphs shown in FIG. 5E.

FIG. 39D shows bar graphs with p-values of graphs shown in FIG. 5E.

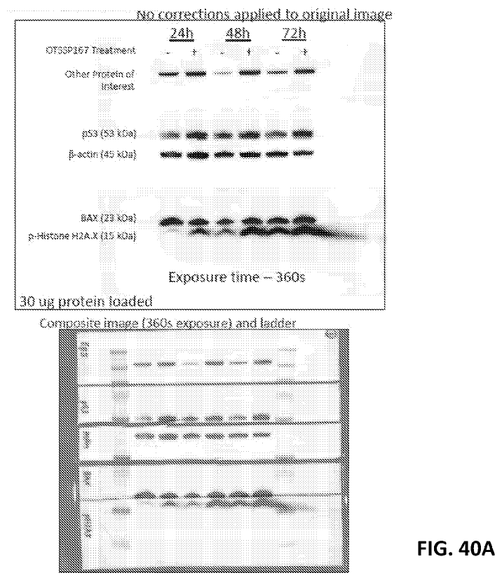

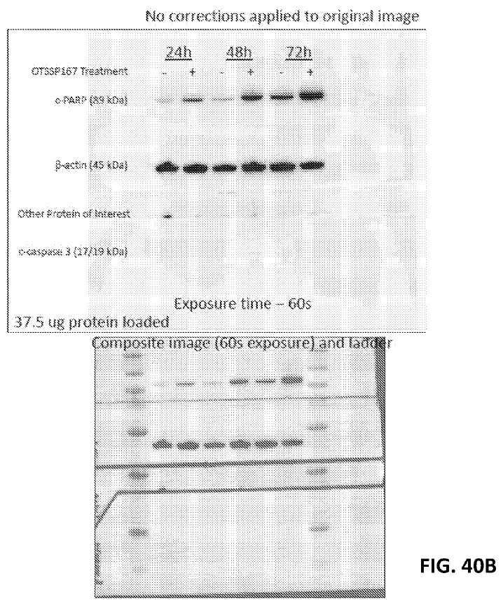

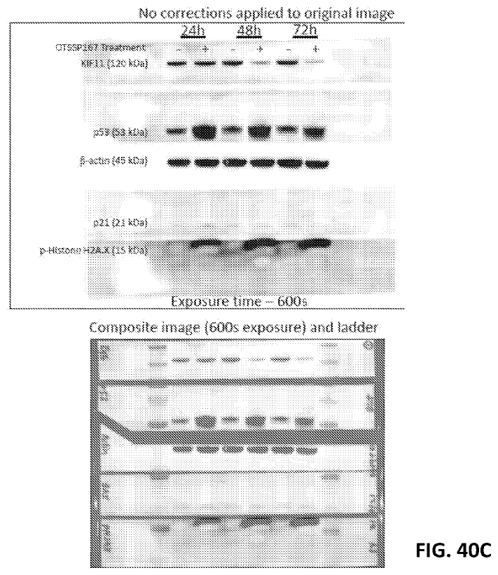

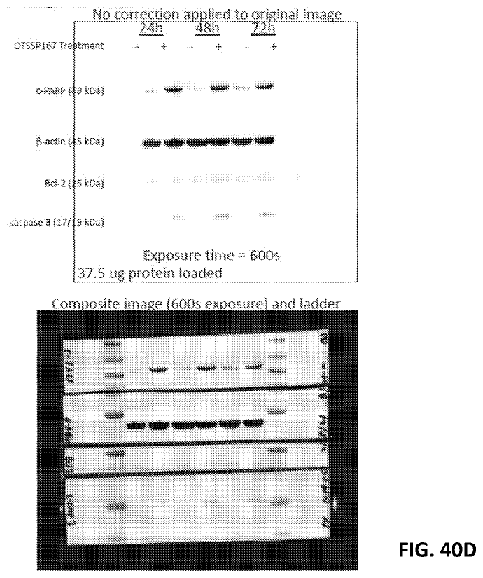

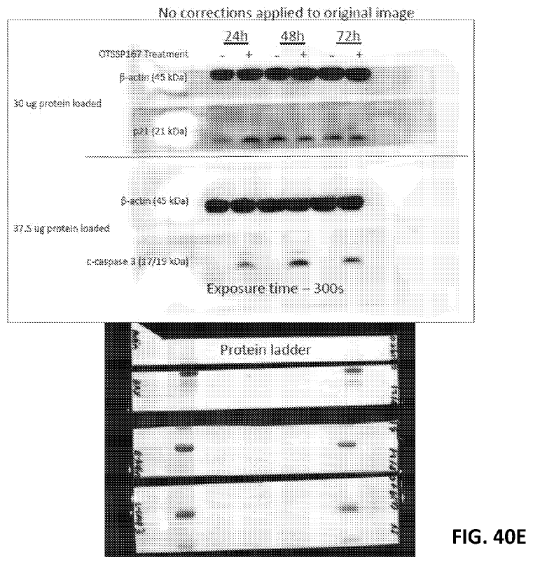

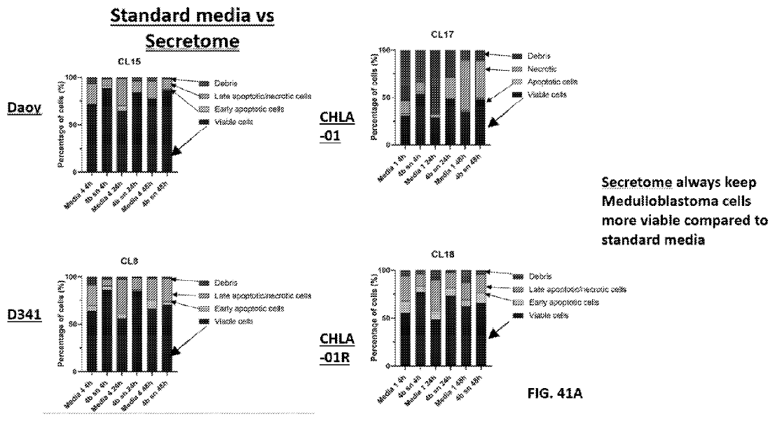

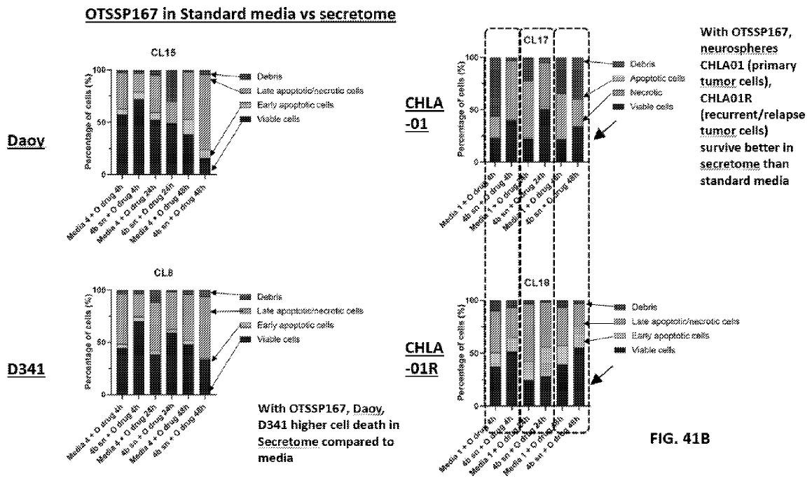

FIG. 40A shows original blot for FIG. 5D Blot 1-CHLA-02.

FIG. 40B shows original blot for FIG. 5D Blot 2-CHLA-02.

FIG. 40C shows original blot for FIG. 5D Blot 1-BT-37.

FIG. 40D shows original blot for FIG. 5D Blot 2-BT-37.

FIG. 40E shows original blot for FIG. 5D Blot 3-BT-37.

FIG. 41A shows bar charts with secretome always keeping medulloblastoma cells more viable compared to standard media.

FIG. 41B shows bar charts with OTSSP167: Daoy, D341 having higher cell death in secretome compared to media. With OTSSP167: neurospheres CHLA01 (primary tumour cells), CHLA01R (recurrent/relapse tumour cells) survive better in secretome than standard media.

FIG. 41C show bar charts for these 3 cell lines (D341, CHLA01, CHLA 01R) treated with ispinesib, cells survive better in secretome than standard media. Gray scale: focus on the bottom-most=viable cells.

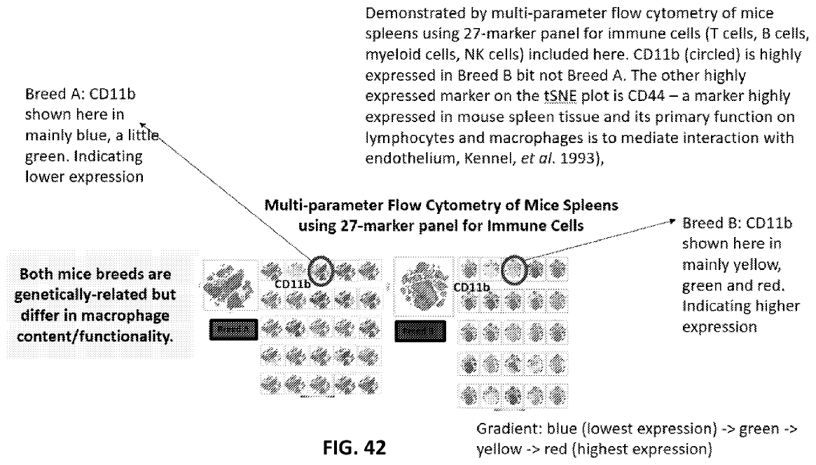

FIG. 42 shows multi-parameter flow cytometry of mice spleens using 27-marker panel for immune cells. Demonstrated by multi-parameter flow cytometry of mice spleens using 27-marker panel for immune cells (T cells, B cells, myeloid cells, NK cells) included here. CD11b (circled) is highly expressed in Breed B but not Breed A. The other highly expressed marker on the tSNE plot is CD44—a marker highly expressed in mouse spleen tissue and its primary function on lymphocytes and macrophages is to mediate interaction with endothelium, Kennel, et al. 1993).

FIG. 43A shows Brain Tumour Type 1 in vivo Data. Patient-derived orthotopic xenograft brain tumour model. Total 33 mice. 17 mice implanted with tumour cells in secretome (Group A). 16 mice implanted with tumour cells in standard media (control. Group B). All in 1 experimental setting. Mice are staged for disease using the staging criteria we previously established (Elghetany, Teo. Scientific Reports 2021). 81.2% in Group B (Tumour+Media) are healthy compared to 58.9% in Group A (Tumour+Secretome). Kaplan Meier Curves: Group A (Mice implanted with Tumour+Secretome): tumours progress faster in this mice & significantly shorter survival compared to Group B (Mice implanted with Tumour+Media). p=0.0054.

FIG. 43B shows survival graph of Brain Tumour Type 2: Medulloblastoma. In this specific molecular subtype of medulloblastoma, there is no statistical difference in survival between Group A (Mice implanted with Tumour+Secretome) and Group B (Mice implanted with Tumour+Media). p=0.3786. Although mice with Tumour+Secretome tend to progress faster. In this same specific molecular subtype of medulloblastoma as Batch 93B, another batch of 21 mice were implanted with Tumor+Secretome in 1 experimental setting (Batch 94B). All 21 mice successfully formed brain tumor. We have previously shown that Ispinesib is an effective drug for Medulloblastoma (Elghetany, Teo. Scientific Reports 2021). Data here shows that Ispinesib is also effective in PDX brain tumors implanted using secretome+Tumor.

FIG. 44 shows a CHLA-02 Southern Blot: Teleblot showing Telomere Length maintained in secretome, comparable to standard media. Effects long lasting to 96 h.

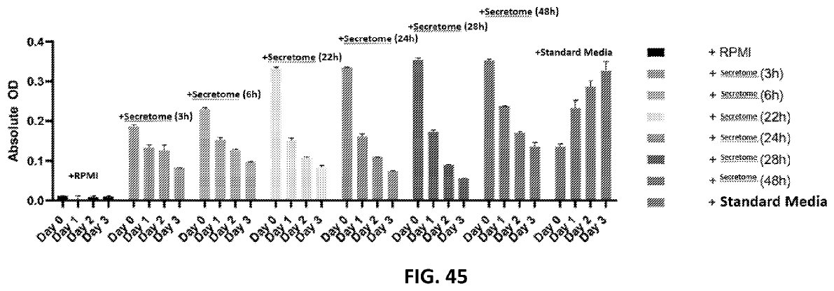

FIG. 45 shows a bar graph with tumour cells displaying highest proliferative abilities at Day 0 when treated with secretome harvested at 22 h to 48 h.

FIG. 46 shows cytokine analyses using cytokine blot comparing supernatant harvested from resistant medulloblastoma tumour cells co-cultured with CD11b+ cells, versus supernatant harvested from CD11b+ conditioned media. CCL2/MCP-1, IL-1Ra, MIP-1a/MIP1B are released in higher quantities when co-cultured in resistant cells.

FIG. 47A shows a volcano plot using fold change of 2, raw p value=0.1, equal variance. Comparing metabolites from Sample 2 (CHLA01+secretome, cultured for 24 h) against Sample 1 (CHLA01+standard media, cultured for 24 h), 29 metabolites were significantly upregulated by tumor cells in secretome (dotted line), 64 metabolites were significantly downregulated by tumor cells in secretome (bold line). CHLA-01. Sample 1 & 2 were run in triplicates. Sample 1: CHLA01+standard media, cultured for 24h. Sample 2: CHLA01 + secretome, cultured for 24 h. Data was normalized using protein concentration and then all data shown are analyzed using MetaboAnalyst 6.0. software. Variance filter of 10% applied on the data set based on Interquantile Range (IQR). Sample normalization by median. Data transformation using log transformation. No data scaling applied.

FIG. 47B shows heatmaps using normalized data, Euclidean distance and ward clustering.

FIG. 47C shows principal component analysis (PCA) plot using 95% confidence interval.

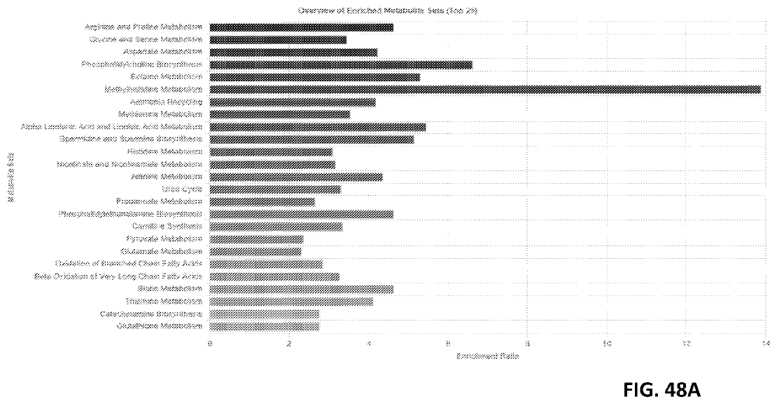

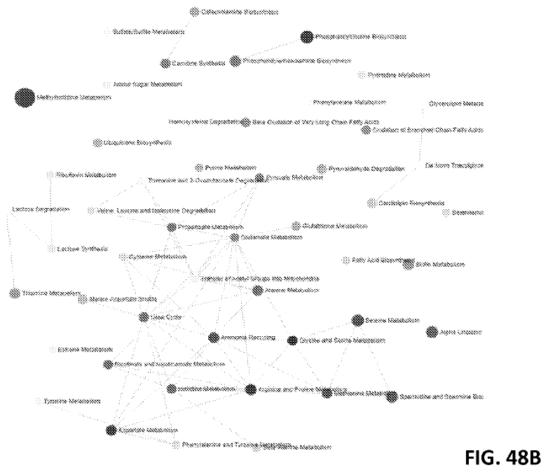

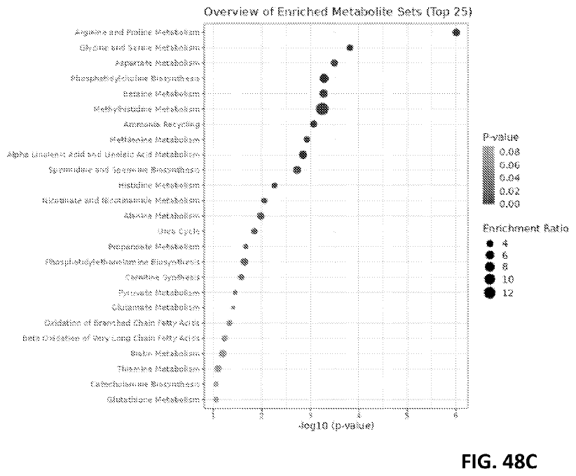

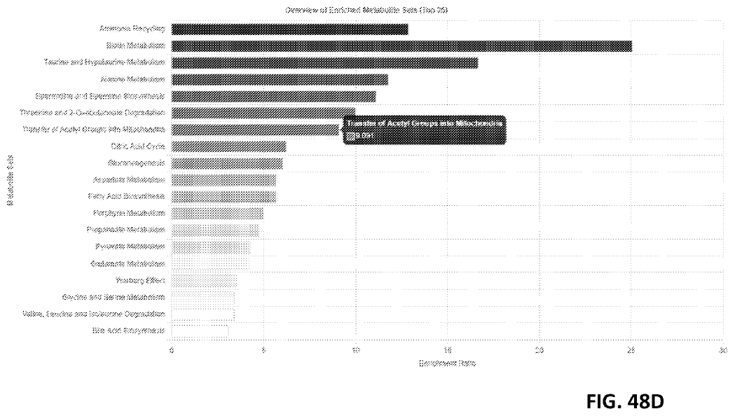

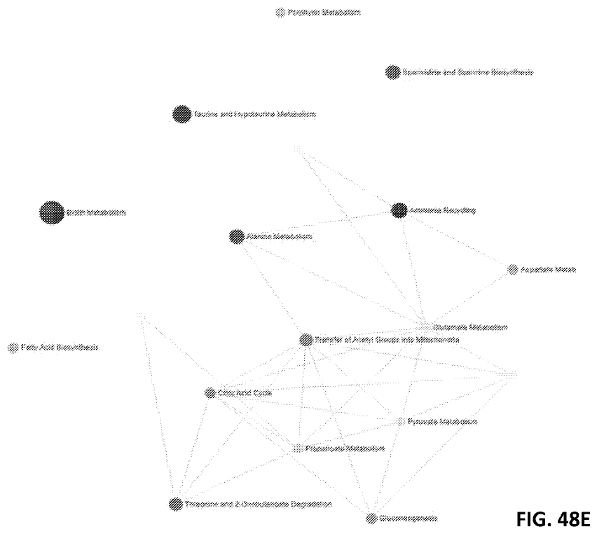

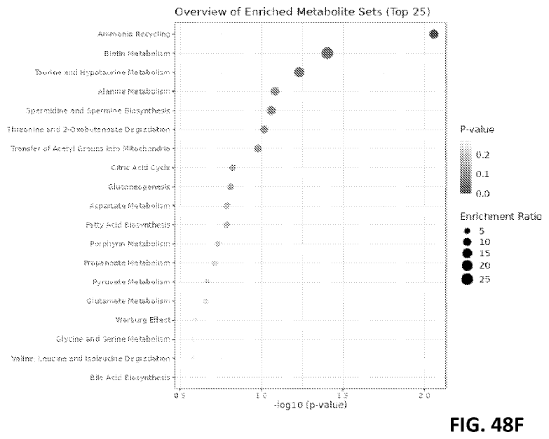

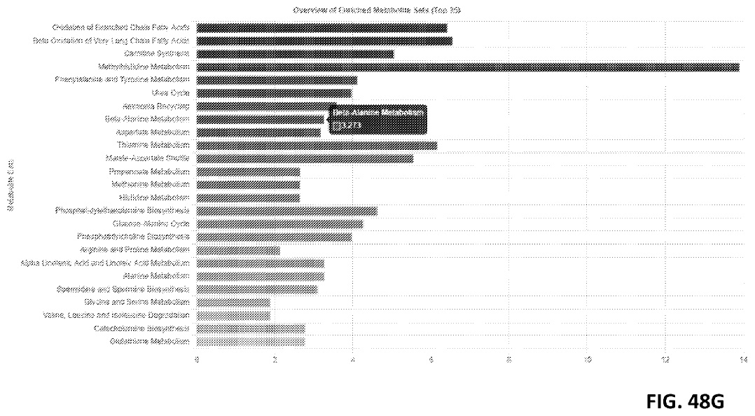

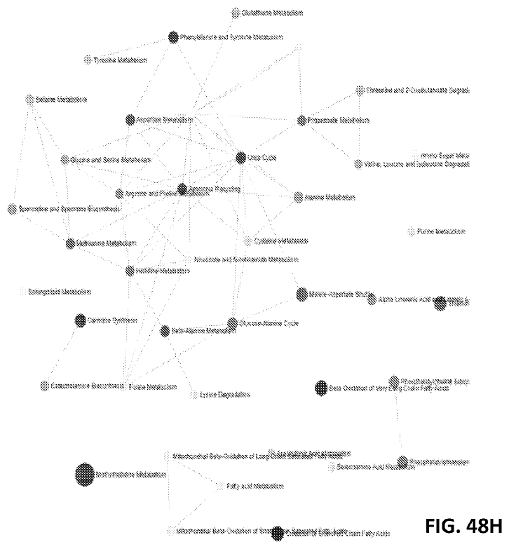

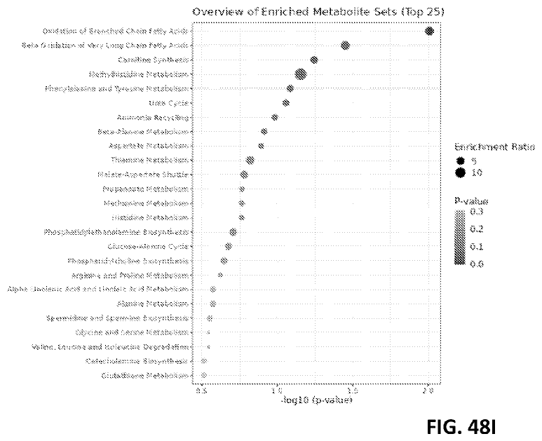

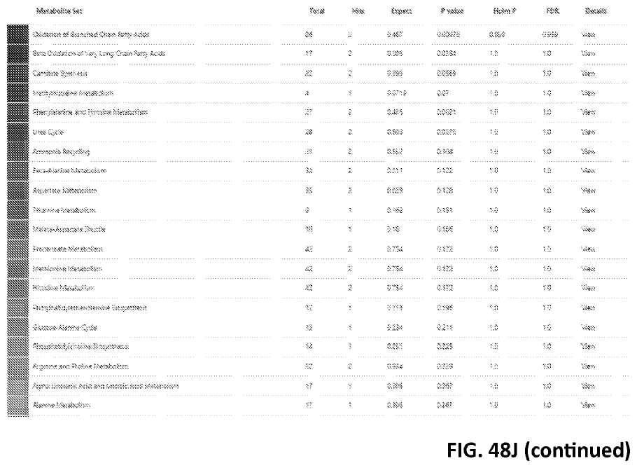

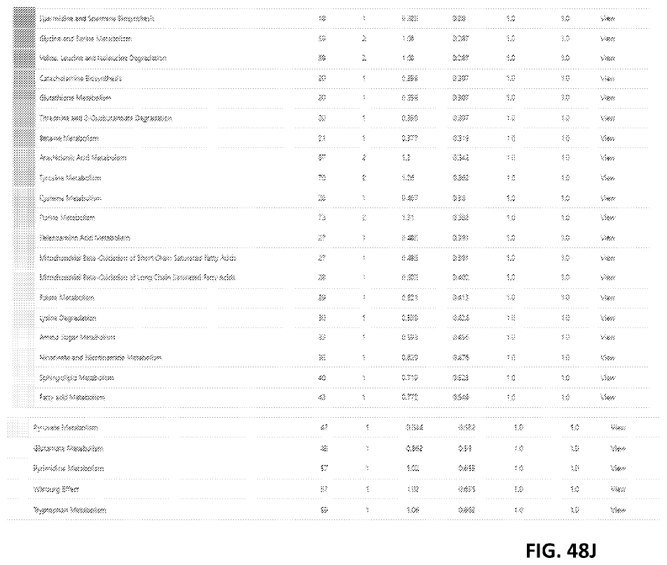

FIG. 48A shows enrichment analyses using all metabolites generated in the experiment of the present disclosure (3 replicates for each of both samples), using SMPDB library set. Top 25 Enriched Metabolite Sets are shown. Methyhistidine Metabolism emerged as the top metabolic pathway for metabolites detected in tumor cells (CHLA01) cultured in both secretome and standard media, for 24 h (Enrichment Ratio<12)

FIG. 48B shows pathway analyses using all metabolites generated in the experiment of the present disclosure (3 replicates for each of both samples) using SMPDB library set. Methyhistidine Metabolism emerged as the top metabolic pathway for metabolites detected in tumor cells (CHLA01) cultured in both secretome and standard media, for 24 h.

FIG. 48C shows pathway analyses using all metabolites generated in the experiment of the present disclosure (3 replicates for each of both samples) using SMPDB library set. Methyhistidine Metabolism emerged as the top metabolic pathway for metabolites detected in tumor cells (CHLA01) cultured in both secretome and standard media, for 24 h.

FIG. 48D shows enrichment analyses shown below using Upregulated metabolites detected in tumor cells (CHLA01) cultured in secretome for 24 h (compared against tumor cells cultured in standard media for 24 h), generated in the experiment of the present disclosure (3 replicates for each of both samples), using SMPDB library set. Top 25 Enriched Metabolite Sets are shown. Biotin Metabolism emerged as the top metabolic pathway upregulated for metabolites detected in tumor cells (CHLA01) cultured in secretome (Enrichment Ratio 25), followed by Taurine and Hypotaurine Metabolism (Enrichment Ratio>15), Ammonia Recycling pathway (Enrichment Ratio<10).

FIG. 48E shows pathway analyses using Upregulated metabolites generated in the experiment of the present disclosure (3 replicates for each of both samples) using SMPDB library set. Biotin Metabolism emerged as the top metabolic pathway for metabolites detected in tumor cells (CHLA01) cultured in secretome, for 24 h, followed by Taurine and Hypotaurine Metabolism, then Ammonia Recycling pathway.

FIG. 48F shows pathway analyses using Upregulated metabolites generated in the experiment of the present disclosure (3 replicates for each of both samples) using SMPDB library set. Biotin Metabolism emerged as the top metabolic pathway upregulated for metabolites detected in tumor cells (CHLA01) cultured in secretome, for 24 h.

FIG. 48G shows enrichment analyses using Downregulated metabolites detected in tumor cells (CHLA01) cultured in secretome for 24 h (compared against tumor cells cultured in standard media for 24 h), generated in the experiment of the present disclosure (3 replicates for each of both samples), using SMPDB library set. Top 25 Enriched Metabolite Sets are shown. Methyhistidine Metabolism emerged as the top metabolic pathway downregulated for metabolites detected in tumor cells (CHLA01) cultured in secretome, for 24 h (Enrichment Ratio<12).

FIG. 48H shows pathway analyses using Downregulated metabolites generated in the experiment of the present disclosure (3 replicates for each of both samples) using SMPDB library set. Methyhistidine Metabolism emerged as the top metabolic pathway downregulated for metabolites detected in tumor cells (CHLA01) cultured in secretome, for 24 h.

FIG. 48I shows pathway analyses shown below using Downregulated metabolites generated in the experiment of the present disclosure (3 replicates for each of both samples) using SMPDB library set. Methyhistidine Metabolism emerged as the top metabolic pathway downregulated for metabolites detected in tumor cells (CHLA01) cultured in secretome, for 24 h.

FIG. 48J shows a table with downregulated metabolites generated using SMPDB library set.

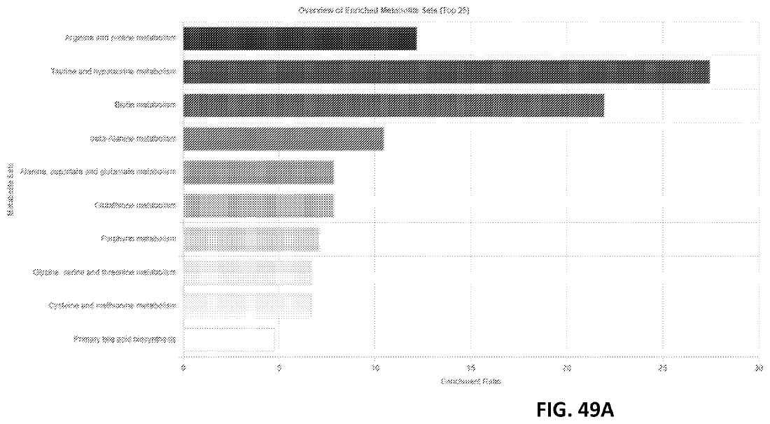

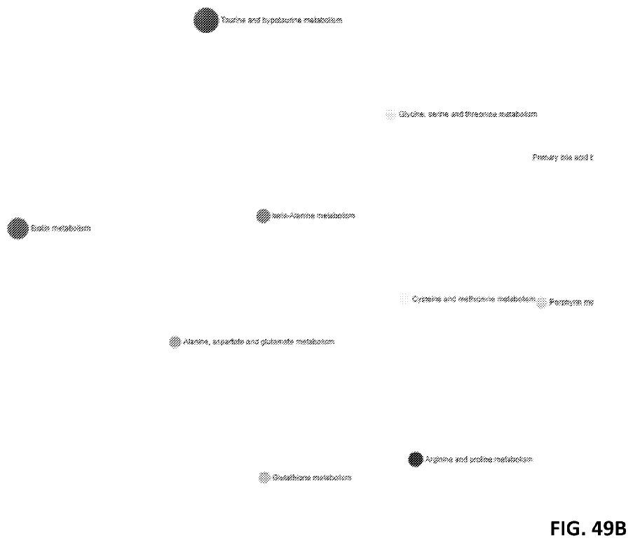

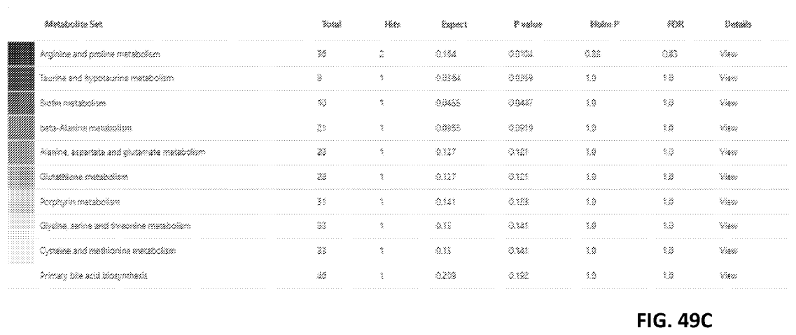

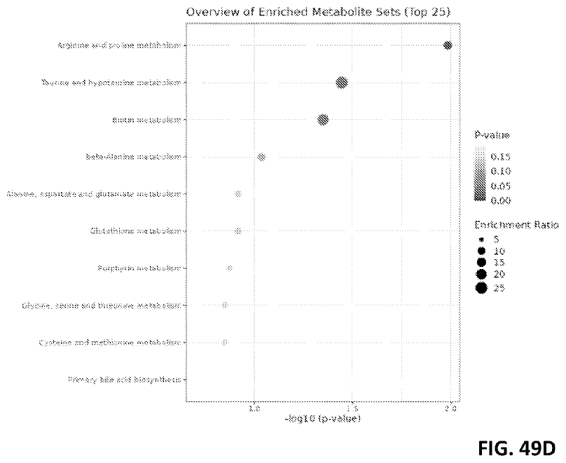

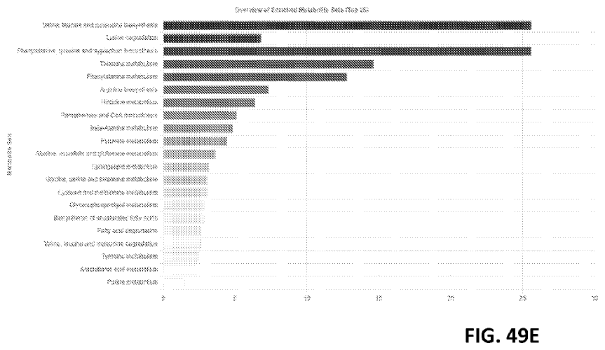

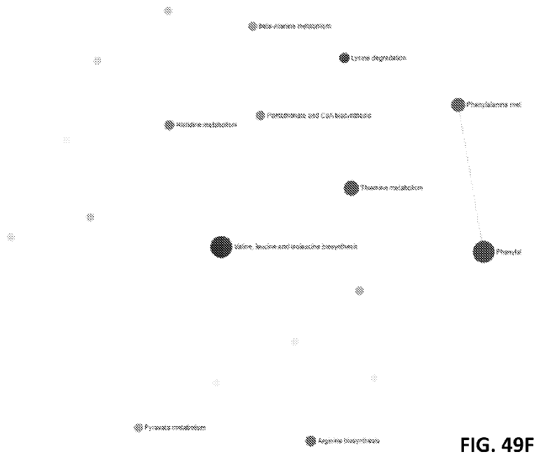

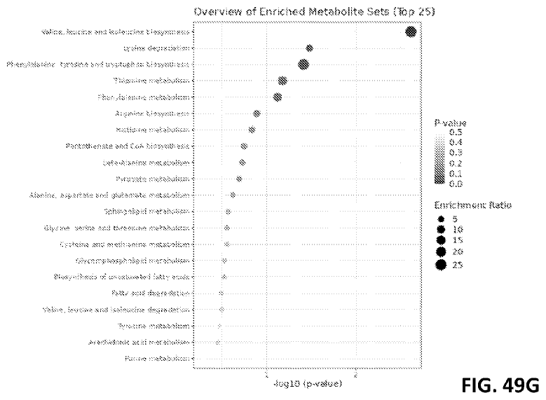

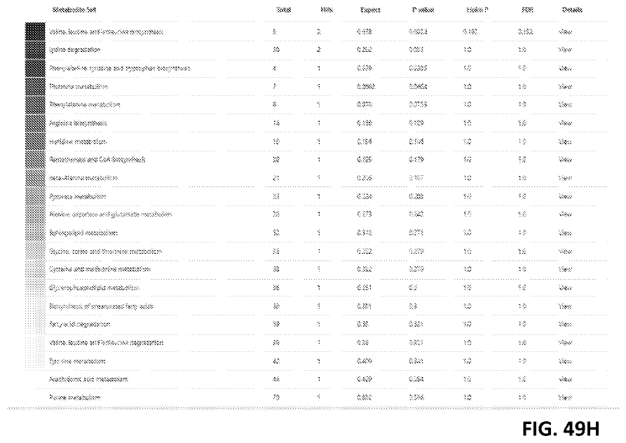

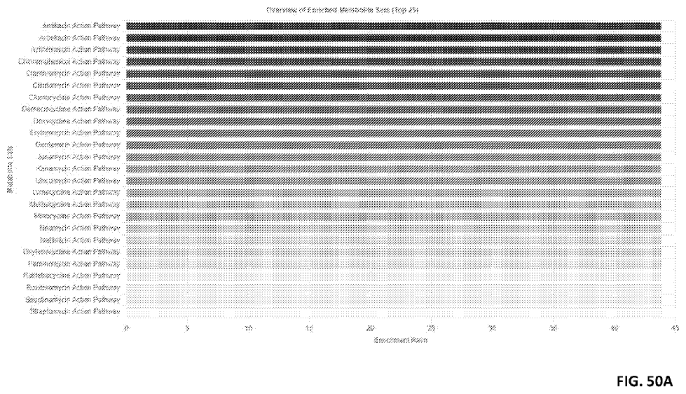

FIG. 49A shows enrichment analyses using Upregulated metabolites detected in tumor cells (CHLA01) cultured in secretome for 24 h (compared against tumor cells cultured in standard media for 24 h), generated in the experiment of the present disclosure (3 replicates for each of both samples), using KEGG library set. Top 25 Enriched Metabolite Sets are shown. Taurine and Hypotaurine Metabolism (Enrichment Ratio>25) emerged as the top metabolic pathway upregulated for metabolites detected in tumor cells (CHLA01) cultured in secretome, followed by Biotin Metabolism (Enrichment Ratio<20).

FIG. 49B to FIG. 49D show pathway analysis of upregulated metabolites generated using the KEGG pathway.

FIG. 49E shows enrichment analyses using Downregulated metabolites detected in tumor cells (CHLA01) cultured in secretome for 24 h (compared against tumor cells cultured in standard media for 24 h), generated in the experiment of the present disclosure (3 replicates for each of both samples), using KEGG library set. Top 25 Enriched Metabolite Sets are shown. Valine, Leucine & Isoleucine Biosynthesis (Enrichment Ratio<25), and Phenylalanine, Tyrosine & Tryptophan Biosynthesis (Enrichment Ratio<25).

FIG. 49F and FIG. 49G show pathway analysis of downregulated metabolites generated using KEGG pathway.

FIG. 49H shows a table of downregulated metabolites generated using KEGG pathway.

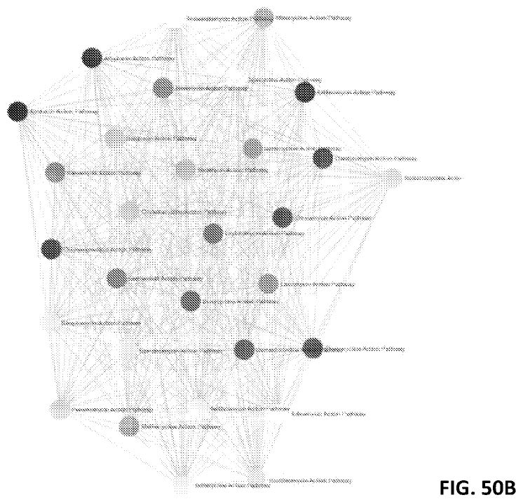

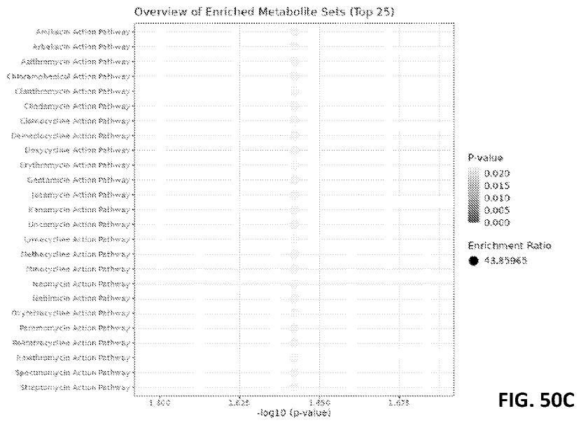

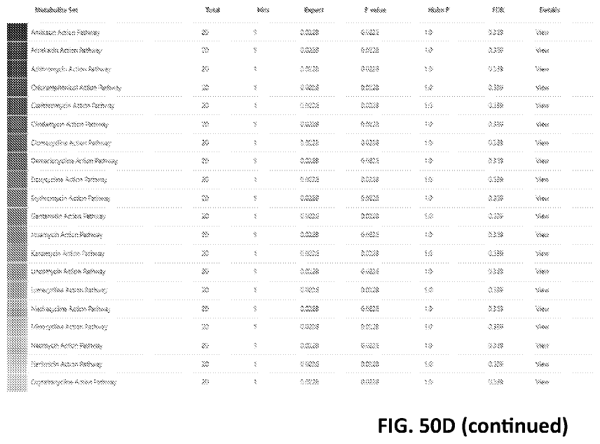

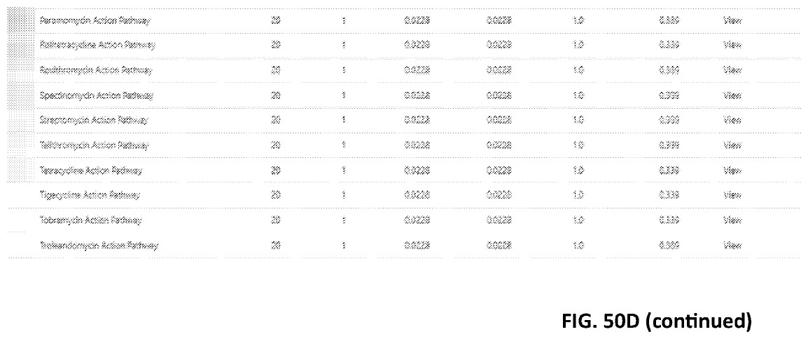

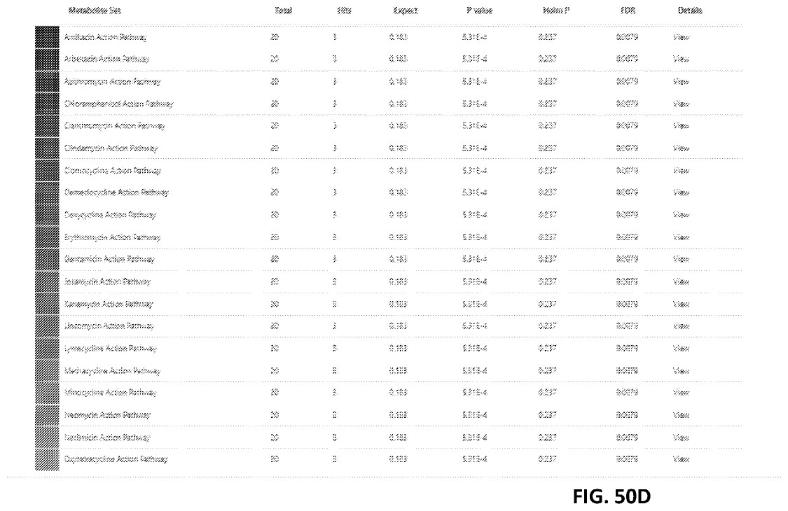

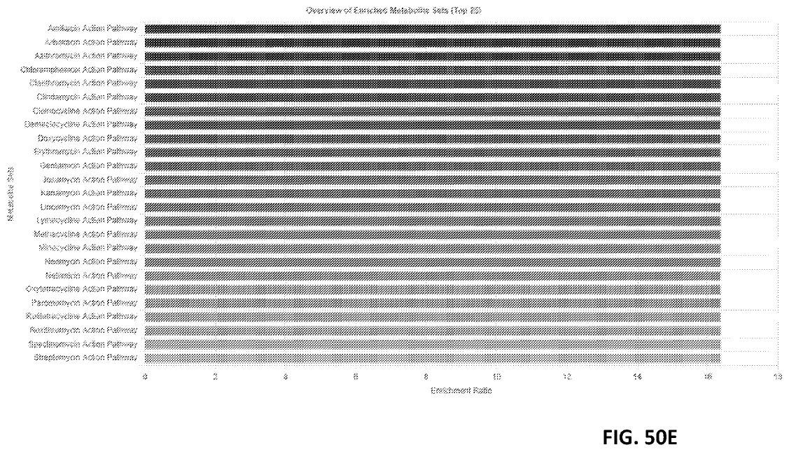

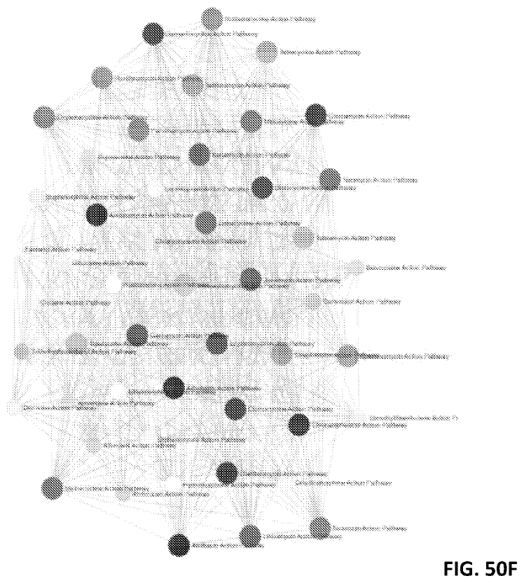

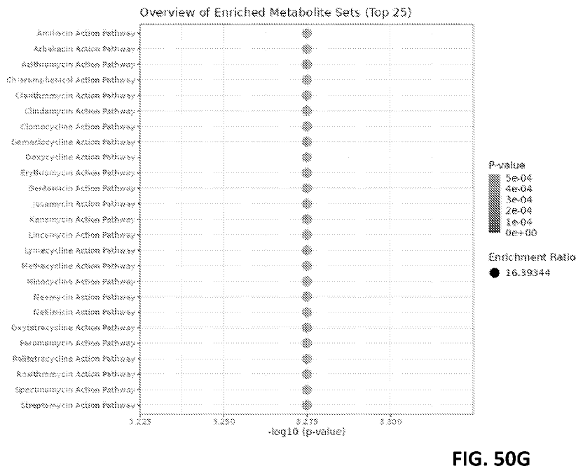

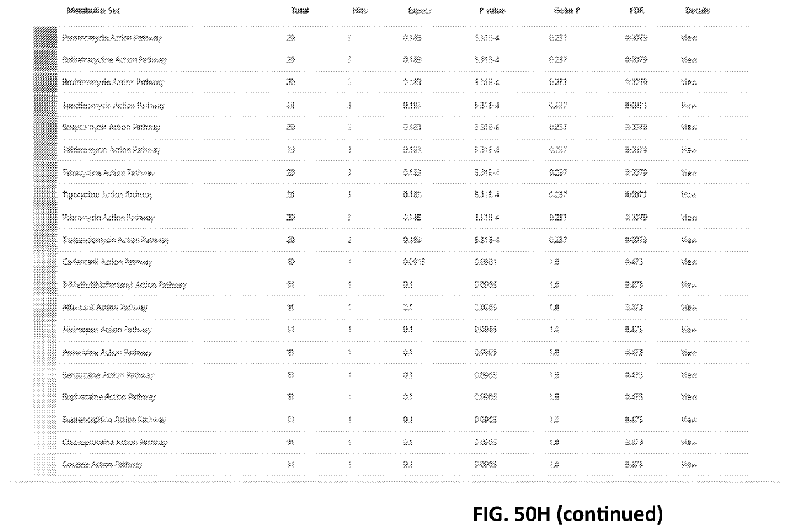

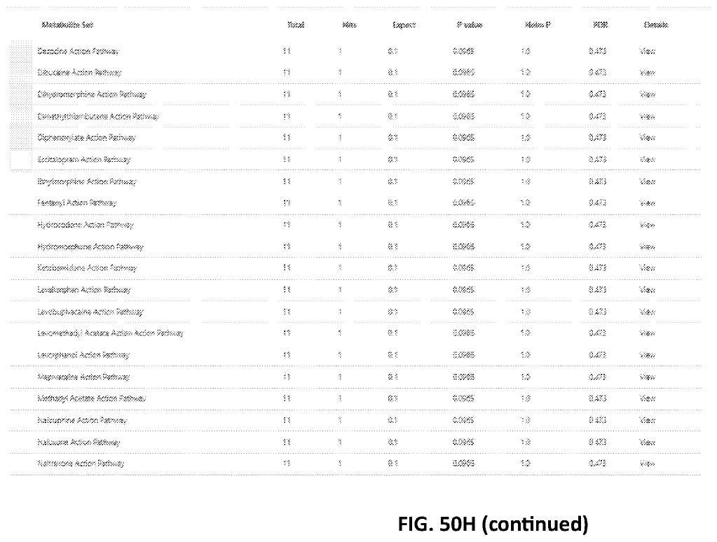

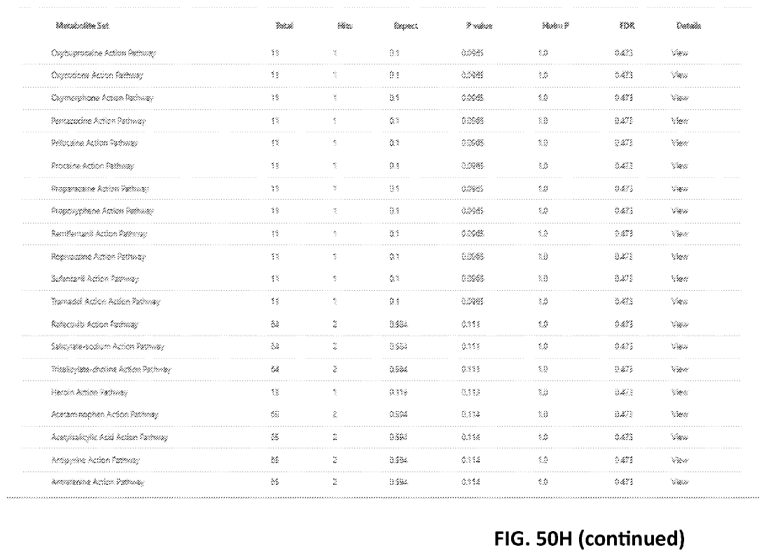

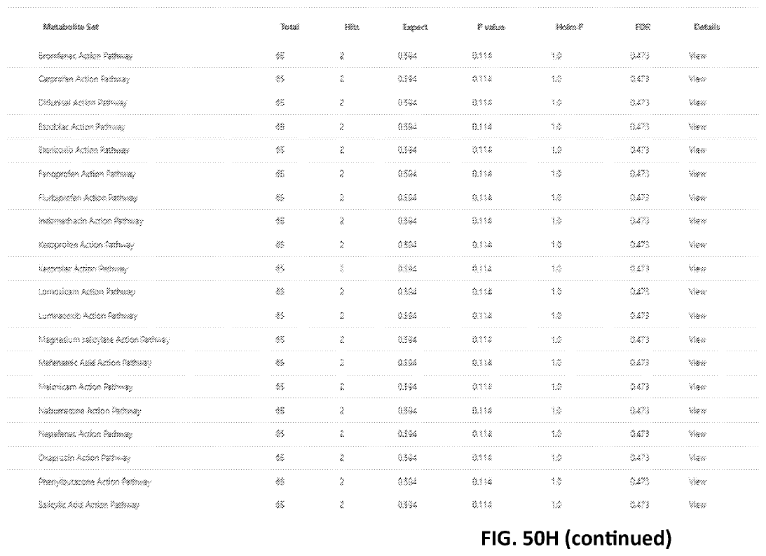

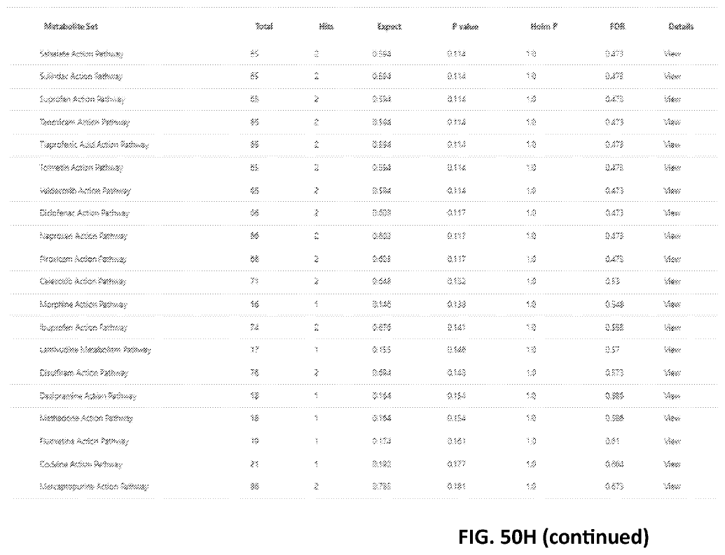

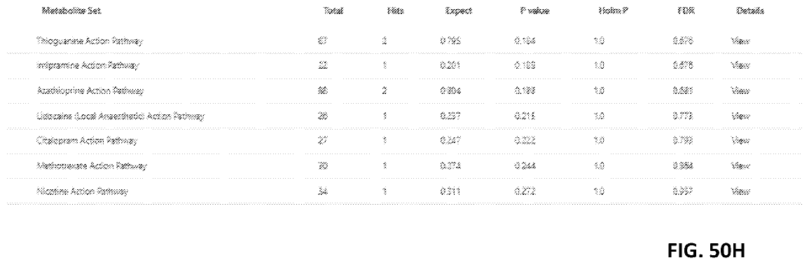

FIG. 50A to 50C show pathway analysis of upregulated metabolites generated using drug related library.

FIG. 50D shows a table of upregulated metabolites generated using drug related library.

FIG. 50E to FIG. 50G show pathway analysis of downregulated metabolites generated using drug related library.

FIG. 50H shows a table of downregulated metabolites generated using drug related library.

FIG. 51A shows a microscopy image (on automated cell counter) of CHLA01R cells retrieved after being cryopreserved in secretome-based freezing media for 65 hours. White circles highlighting mainly viable cells.

FIG. 51B shows microscopy Image (on automated cell counter) of CHLA01R cells retrieved after being cryopreserved in secretome-based freezing media for 188 hours. White circles highlighting mainly viable cells. Cell number and viability of the vial retrieved after 188 h. Cell number is even higher at 3 million (retrieval), compared to what was frozen (2.49 million). Viability dropped ~15% from day of freezing, which is comparable/superior to standard-media based freezing media)

### APPLICATIONS

Embodiments as disclosed herein provide a composition comprising a secretome obtained by culturing a cell in the presence of an agent to generate an immune cell.

Advantageously, the present disclosure is scalable. The secretome of the present disclosure can be harvested in large quantities upon culturing an immune progenitor cell with an agent for 48 hours, packaged in cryovials and is stable in −80° C. storage conditions for 3 years or more.

More advantageously, the present disclosure allows screening of patient tumours to tailor precision drugs and select suitable patients with immune rich tumours for clinical trials. The secretome product of the present disclosure enables testing of new drugs/drug libraries in-vitro for high-throughput drug discovery, under immune rich condition of cancer cells. This can be done in a laboratory setting prior to application in patients during a clinical trial and is useful for pharmaceutical industries.

Even more advantageously, the present disclosure showed that the secretome of the present disclosure sustain cells during nutrient stress and have implications in relapse subtypes of an aggressive childhood brain tumour (ATRT). The secretome product of the present disclosure is also found in other adult cancer cell types and can be applied to other non-cancer human disease such as chronic inflammation, ulcerative colitis, Crohn's disease, Alzheimer's disease, dementia, coronavirus disease, HIV, which may share this secretome.

Even more advantageously, the secretome of the present disclosure can be used for cryopreservation of cells (such as cancer cells, healthy cells). The secretome of the present disclosure can preserve cells such as eggs, sperm, cord blood.

Even more advantageously, the secretome of the present disclosure can be used as cell culture media and in cell studies for biomarker discovery. The secretome of the present disclosure provides immune rich conditions for laboratory testing of diseased cells, testing of new drugs on cancer cells or screening of compounds to identify drugs effective against diseased cells/cancer cells in an immune-rich in-vitro culture, mimicking microenvironment conditions in patient tumours.

Even more advantageously, the secretome of the present disclosure can be used as a freezing media. Cells that have been frozen with secretome freezing media (90% secretome and 10% DMSO) have been able to maintain or even regain viability as compared to standard freezing media (standard media and 10% DMSO).

Even more advantageously, the secretome of the present disclosure can be used in therapeutic applications such as tissue regeneration (such as skin regeneration/skin graft manufacturing, nerve/brain regeneration/anti-aging, liver regeneration, vascular regeneration, regeneration of tissues/organs for transplant), wound healing, angiogenesis, osteogenesis, treatment of inflammatory response and/or drug discovery.

Even more advantageously, the present disclosure shows the screening capability to identify secretome phenotypes for each cancer type to develop personalized therapies for precision medicine.

Even more advantageously, the present disclosure provides a pan-cancer secretome atlas that includes a database of a plurality of secretome phenotype of a plurality of disease which includes cancer, acute/chronic inflammation disease. The secretome atlas of the present disclosure can be used to select specific cancer types to test new drugs/chemical agents using the secretome product screen of the present disclosure.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. A composition comprising a secretome obtained by culturing an immune progenitor cell in the presence of an agent that activates the immune progenitor cell to an activated immune cell.

2. The composition according to claim 1, wherein the secretome comprises a protein comprising a cytokine, a hormone, an antibody, a growth factor, an extracellular matrix protein, a shed receptor, a coagulation factor, an adhesion molecule, a protease, a kinase and/or a glycoprotein.

3. The composition according to claim 1, wherein the secretome comprises one or more factors selected from the group consisting of CCL2/MCP-1, MIP-1α/MIP-1β, CCL5/RANTES, MIF, IL-1ra/IL-IF3, IL-8 and Serpin E1/PAI-1.

4. The composition according to claim 1, wherein the immune cell comprises a myeloid cell, a lymphoid cell, and/or an innate immune cell.

5. The composition according to claim 1, wherein the immune progenitor cell is a myeloid progenitor cell.

6. The composition according to claim 1, wherein the immune cell is a macrophage expressing CD11b and/or CD163.

7. The composition according to claim 1, wherein the secretome is a macrophage-derived secretome.

8. The composition according to claim 1, wherein the agent comprises a stimulating agent or a suppressing agent.

9. The composition according to claim 1, wherein the agent is a myeloid stimulating agent.

10. The composition according to claim 1, wherein the agent is PMA and/or IL-4.

11. The composition according to claim 1, wherein the secretome sustains cell growth, cell proliferation, cryopreserve cells, elongates telomere length in cancer cells, involved in metabolic reprogramming, involved in drug screening and drug discovery, identifying drug resistance and/or preventing cancer cell death under nutrient stress conditions.

12. (canceled)

13. A method of generating a macrophage-derived secretome comprising culturing an immune progenitor cell in the presence of a stimulating agent to thereby generate an activated immune cell population and harvesting the secretome from the generated activated immune cell population.

14. The method according to claim 13 further comprising culturing the activated immune cell in the presence of a diseased cell.

15. The method according to claim 13, wherein the diseased cell is a cell from a proliferative disease.

16. The method according to claim 13, further comprising culturing the activated immune cell in the presence of a diseased cell wherein the diseased cell is a brain tumour, an atypical teratoid rhabdoid tumour (ATRT), glioblastoma multiforme, and/or medulloblastoma.

17. A method of screening a drug comprising culturing the drug with a diseased cell in the presence of the composition of claim 1.

18. (canceled)

19. The method of claim 13 further comprising culturing the activated immune cell in the presence of a proliferative cell.

20. A method of enhancing or initiating tissue regeneration, the method comprising culturing an immune progenitor cell in the presence of a stimulating agent to thereby generate an activated immune cell population;
harvesting secretome from the generated activated immune cell population; and
administering the harvested secretome to the tissue in need of tissue regeneration.

21. The method of claim 20, wherein the tissue is a skin tissue, a nerve/brain, a spinal cord, a vascular tissue, a cardiovascular tissue, a heart tissue, a liver tissue, a chondrocyte/cartilage, or a corneal tissue.

22. The method of claim 20, wherein the method comprises the step of cryopreserving the tissue in the harvested secretome.
